(19) United States
(12) Patent Application Publication
(10) Pub. No.: US 2017/0256722 A1
(43) Pub. Date: Sep. 7, 2017

(54) A HOLE TRANSPORT MATERIAL AND AN ORGANIC ELECTROLUMINESCENT DEVICE COMPRISING THE SAME

(71) Applicant: Rohm and Haas Electronic Materials Korea Ltd., Cheonan (KR)

(72) Inventors: Jae-Hoon Shim, Seoul (KR); Kyoung-Jin Park, Seongnam (KR); Tae-Jin Lee, Seoul (KR); Hee-Choon Ahn, Seoul (KR); Doo-Hyeon Moon, Hwaseong (KR); Ji-Song Jun, Hwaseong (KR); Jin-Ri Hong, Cheonan (KR); Yoo-Jin Doh, Seoul (KR)

(21) Appl. No.: 15/506,277

(22) PCT Filed: Sep. 4, 2015
(86) PCT No.: PCT/KR2015/009376

§ 371 Date: Feb. 24, 2017

(30) Foreign Application Priority Data
Sep. 5, 2014 (KR) 10-2014-0118870

(51) Int. Cl. H01L 51/00; C07D 405/10; C07D 409/10; C07D 209/86

(52) U.S. Cl. CPC H01L 51/0072; C07D 209/86; H01L 51/0052; C07D 405/10; H01L 51/0073; C07D 409/10; H01L 51/0074; H01L 51/0058; H01L 51/506

(57) ABSTRACT

The present invention relates to a hole transport material and an organic electroluminescent device comprising the same. By using the hole transport material according to the present invention, an organic electroluminescent device having significantly improved operational lifespan while maintaining low driving voltage and high current and power efficiencies can be produced.

### TECHNICAL FIELD

The present invention relates to a hole transport material and an organic electroluminescent device comprising the same.

### BACKGROUND ART

An electroluminescent device (EL device) is a self-light-emitting device which has advantages in that it provides a wider viewing angle, a greater contrast ratio, and a faster response time. An organic EL device was first developed by Eastman Kodak, by using small aromatic diamine molecules, and aluminum complexes as materials for forming a light-emitting layer [Appl. Phys. Lett. 51, 913, 1987].

The most important factor determining luminous efficiency in an organic EL device is the light-emitting material. Until now, fluorescent materials have been widely used as a light-emitting material. However, in view of electroluminescent mechanisms, since phosphorescent materials theoretically enhance luminous efficiency by four (4) times compared to fluorescent materials, development of phosphorescent light-emitting materials are widely being researched. Iridium(III) complexes have been widely known as phosphorescent materials, including bis(2-(2′-benzothienyl)-pyridinato-N,C3′)iridium(acetylacetonate) ((acac)Ir(btp)₂), tris(2-phenylpyridine)iridium (Ir(ppy)₃) and bis(4,6-difluorophenylpyridinato-N,C2)picolinate iridium (Firpic) as red, green and blue materials, respectively.

At present, 4,4′-N,N′-dicarbazol-biphenyl (CBP) is the most widely known phosphorescent host materials. Recently, Pioneer (Japan) et al. developed a high performance organic EL device using bathocuproine (BCP) and aluminum(III)bis(2-methyl-8-quinolinate)(4-phenylphenolate) (BAlq) etc., as host materials, which were known as hole blocking layer materials.

Although these materials provide good light-emitting characteristics, they have the following disadvantages: (1) Due to their low glass transition temperature and poor thermal stability, their degradation may occur during a high-temperature deposition process in a vacuum, and the lifespan of the device decreases. (2) The power efficiency of an organic EL device is given by [(π/voltage)× current efficiency], and the power efficiency is inversely proportional to the voltage. Although an organic EL device comprising phosphorescent host materials provides higher current efficiency (cd/A) than one comprising fluorescent materials, a significantly high driving voltage is necessary. Thus, there is no merit in terms of power efficiency (Im/W). (3) Further, the operational lifespan of an organic EL device is short and luminous efficiency is still required to be improved.

Meanwhile, in order to enhance its efficiency and stability, an organic EL device has a structure of a multilayer comprising a hole injection layer, a hole transport layer, a light-emitting layer, an electron transport layer, and an electron injection layer. The selection of a compound comprised in the hole transport layer is known as a method for improving the characteristics of a device such as hole transport efficiency to the light-emitting layer, luminous efficiency, lifespan, etc.

In this regard, copper phthalocyanine (CuPc), 4,4′-bis[N-(1-naphthyl)-N-phenylamino]biphenyl (NPB), N,N′-diphenyl-N,N′-bis(3-methylphenyl)-(1,1′-biphenyl)-4,4′-diamine (TPD), 4,4′,4″-tris(3-methylphenylphenylamino)triphenylamine (MTDATA), etc., were used as a hole injection and transport material. However, an organic EL device using these materials has problems of reduction in quantum efficiency and operational lifespan. It is because, when an organic EL device is driven under high current, thermal stress occurs between an anode and a hole injection layer. Such thermal stress significantly reduces the operational lifespan of the device. Further, since the organic material used in the hole injection layer has very high hole mobility, the hole-electron charge balance may be broken and quantum yield (cd/A) may decrease.

Therefore, a hole transport layer for improving durability of an organic EL device still needs to be developed.

Korean Patent Appln. Laying-Open No. 10-2010-0079458 discloses a bis-carbazole compound as an organic electroluminescent compound. However, the organic electroluminescent device of the above reference does not show satisfactory device lifespan.

### DISCLOSURE OF THE INVENTION

### Problems to be Solved

The objective of the present invention is to solve the problem of lifespan decrease due to interfacial light emission between the hole transport layer and the light-emitting layer, and provide an organic electroluminescent device having excellent operational efficiency and long operational lifespan.

### Solution to Problems

The present inventors found that the above objective can be achieved by an organic electroluminescent compound represented by the following formula 1:

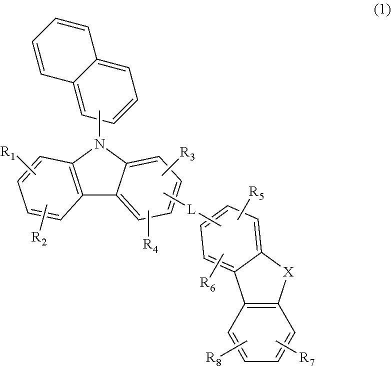

wherein

X represents O, S, CR₉R₁₀, or NR₁₁;

L represents a single bond, or a substituted or unsubstituted (C6-C30)arylene;

R₁ to R₁₁ each independently represent hydrogen, deuterium, a substituted or unsubstituted (C1-C30)alkyl, a substituted or unsubstituted (C6-C30)aryl, a substituted or unsubstituted 3- to 30-membered heteroaryl, a substituted or unsubstituted (C3-C30)cycloalkyl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or are linked to each other to form a mono- or polycyclic, (C3-C30) alicyclic or aromatic ring, whose carbon atom(s) may be replaced with at least one hetero atom selected from nitrogen, oxygen, and sulfur; and

the heteroaryl contains at least one hetero atom selected from B, N, O, S, Si, and P.

### Effects of the Invention

By using the hole transport material according to the present invention, the problem of lifespan decrease due to interfacial light emission between the hole transport layer and the light-emitting layer, and the organic electroluminescent device shows excellent operational efficiency and long operational lifespan.

### EMBODIMENTS OF THE INVENTION

Hereinafter, the present invention will be described in detail. However, the following description is intended to explain the invention, and is not meant in any way to restrict the scope of the invention.

According to one embodiment of the present invention, a hole transport material comprising a compound represented by formula 1 is provided. The hole transport material can be a mixture or composition which further comprises conventional materials generally used in producing organic electroluminescent devices.

In order to perform electron blocking which is the main characteristic of a hole transport layer, anion stability is required. By introducing naphthalene (aryl group) etc., to the conventional hole transport layer, the anion stability of a hole transport layer is improved, which can provide an effect of preventing lifespan decrease due to interfacial light emission.

The compound represented by the above formula 1 will be described in detail.

Herein, “(C1-C30)alkyl” indicates a linear or branched alkyl chain having 1 to 30, preferably 1 to 10, and more preferably 1 to 6 carbon atoms constituting the chain, and includes methyl, ethyl, n-propyl, isopropyl, n-butyl, isobutyl, tert-butyl, etc. “(C2-C30) alkenyl” indicates a linear or branched alkenyl chain having 2 to 30, preferably 2 to 20, and more preferably 2 to 10 carbon atoms constituting the chain and includes vinyl, 1-propenyl, 2-propenyl, 1-butenyl, 2-butenyl, 3-butenyl, 2-methylbut-2-enyl, etc. “(C2-C30)alkynyl” indicates a linear or branched alkynyl chain having 2 to 30, preferably 2 to 20, and more preferably 2 to 10 carbon atoms constituting the chain and includes ethynyl, 1-propynyl, 2-propynyl, 1-butynyl, 2-butynyl, 3-butynyl, 1-methylpent-2-ynyl, etc. “(C3-C30)cycloalkyl” indicates a mono- or polycyclic hydrocarbon having 3 to 30, preferably 3 to 20, and more preferably 3 to 7 ring backbone carbon atoms and includes cyclopropyl, cyclobutyl, cyclopentyl, cyclohexyl, etc. “3- to 7-membered heterocycloalkyl” indicates a cycloalkyl having 3 to 7 ring backbone atoms including at least one hetero atom selected from B, N, O, S, Si, and P, preferably O, S, and N, and includes tetrahydrofuran, pyrrolidine, thiolan, tetrahydropyran, Furthermore, “(C6-C30)aryl(ene)” indicates a monocyclic or fused ring-based radical derived from an aromatic hydrocarbon and having 6 to 30, preferably 6 to 20, and more preferably 6 to 15 ring backbone carbon atoms, and includes phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, etc. “3- to 30-membered heteroaryl(ene)” indicates an aryl group having 3 to 30 ring backbone atoms including at least one, preferably 1 to 4, hetero atom selected from the group consisting of B, N, O, S, Si, and P; may be a monocyclic ring, or a fused ring condensed with at least one benzene ring; may be partially saturated; may be one formed by linking at least one heteroaryl or aryl group to a heteroaryl group via a single bond(s); and includes a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzonaphthothiophenyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl, etc. Furthermore, “halogen” includes F, Cl, Br, and I.

Herein, “substituted” in the expression, “substituted or unsubstituted,” means that a hydrogen atom in a certain functional group is replaced with another atom or group, i.e. a substituent. In the present invention, the substituents of the substituted (C1-C30)alkyl, the substituted (C3-C30)cycloalkyl, the substituted (C6-C30)aryl(ene), the substituted 3- to 30-membered heteroaryl, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di-(C1-C30)alkylamino, the substituted mono- or di-(C6-C30)arylamino, and the substituted (C1-C30)alkyl(C6-C30)arylamino in L, and R₁ to R₁₁ in formula 1 each independently are at least one selected from the group consisting of deuterium, a halogen, a cyano, a carboxyl, a nitro, a hydroxyl, a (C1-C30)alkyl, a halo(C1-C30)alkyl, a (C2-C30) alkenyl, a (C2-C30) alkynyl, a (C1-C30)alkoxy, a (C1-C30)alkylthio, a (C3-C30)cycloalkyl, a (C3-C30)cycloalkenyl, a 3- to 7-membered heterocycloalkyl, a (C6-C30)aryloxy, a (C6-C30)arylthio, a 3- to 30-membered heteroaryl unsubstituted or substituted with a (C6-C30)aryl, a (C6-C30)aryl unsubstituted or substituted with a 3- to 30-membered heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl, and preferably a (C6-C15)aryl.

In formula 1 above, X represents O, S, CR₉R₁₀, or NR₁₁

L represents a single bond, or a substituted or unsubstituted (C6-C30)arylene, preferably represents a single bond, or a substituted or unsubstituted (C6-C12)arylene, and more preferably represents a single bond, or an unsubstituted (C6-C12)arylene.

R₁ to R₁₁ each independently represent hydrogen, deuterium, a substituted or unsubstituted (C1-C30)alkyl, a substituted or unsubstituted (C6-C30)aryl, a substituted or unsubstituted 3- to 30-membered heteroaryl, a substituted or unsubstituted (C3-C30)cycloalkyl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or are linked to each other to form a mono- or polycyclic, (C3-C30) alicyclic or aromatic ring, whose carbon atom(s) may be replaced with at least one hetero atom selected from nitrogen, oxygen, and sulfur.

Preferably, R₁ to R₉ each independently represent hydrogen, or a substituted or unsubstituted 5- to 15-membered heteroaryl; or are linked to each other to form a mono- or polycyclic, (C5-C15) alicyclic or aromatic ring, and more preferably each independently represent hydrogen, or a 5- to 15-membered heteroaryl unsubstituted or substituted with a (C6-C12)aryl; or are linked to each other to form a monocyclic, (C5-C15) aromatic ring.

Preferably, R₉ to R₁₁ each independently represent hydrogen, a substituted or unsubstituted (C1-C6)alkyl, or a substituted or unsubstituted (C6-C15)aryl; or are linked to each other to form a mono- or polycyclic, (C5-C15) alicyclic or aromatic ring, and more preferably each independently represent hydrogen, an unsubstituted (C1-C6)alkyl, or an unsubstituted (C6-C15)aryl; or are linked to each other to form a polycyclic, (C5-C15) aromatic ring.

According to one embodiment of the present invention, in formula 1 above, X represents O, S, CR₉R₁₀, or NR₁₁; L represents a single bond, or a substituted or unsubstituted (C6-C12)arylene; R₁ to R₈ each independently represent hydrogen, or a substituted or unsubstituted 5- to 15-membered heteroaryl; or are linked to each other to form a mono- or polycyclic, (C5-C15) alicyclic or aromatic ring; and R₉ to R₁₁ each independently represent hydrogen, a substituted or unsubstituted (C1-C6)alkyl, or a substituted or unsubstituted (C6-C15)aryl; or are linked to each other to form a mono- or polycyclic, (C5-C15) alicyclic or aromatic ring.

According to another embodiment of the present invention, in formula 1 above, X represents O, S, CR₉R₁₀, or NR₁₁; L represents a single bond, or an unsubstituted (C6-C12)arylene; R₁ to R₈ each independently represent hydrogen, or a 5- to 15-membered heteroaryl unsubstituted or substituted with a (C6-C12)aryl; or are linked to each other to form a monocyclic, (C5-C15) aromatic ring; and R₉ to R₁₁ each independently represent hydrogen, an unsubstituted (C1-C6)alkyl, or an unsubstituted (C6-C15)aryl; or are linked to each other to form a polycyclic, (C5-C15) aromatic ring.

The compound represented by formula 1 includes the following compounds, but are not limited thereto:

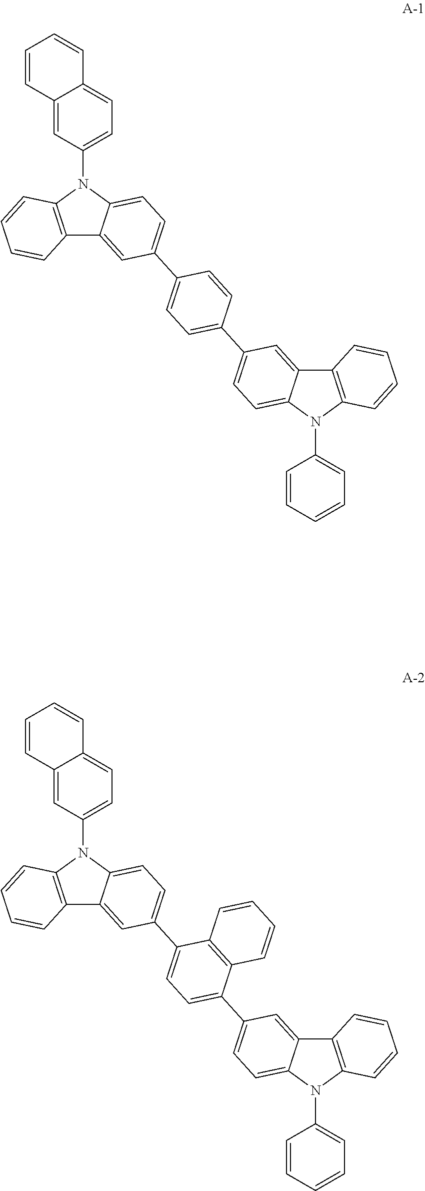 [SMILES: c1ccc(-n2c3ccccc3c3cc(-c4ccc(-c5ccc6c(c5)c5ccccc5n6-c5ccc6ccccc6c5)cc4)ccc32)cc1 ; c1ccc(-n2c3ccccc3c3cc(-c4ccc(-c5ccc6c(c5)c5ccccc5n6-c5ccc6ccccc6c5)c5ccccc45)ccc32)cc1]

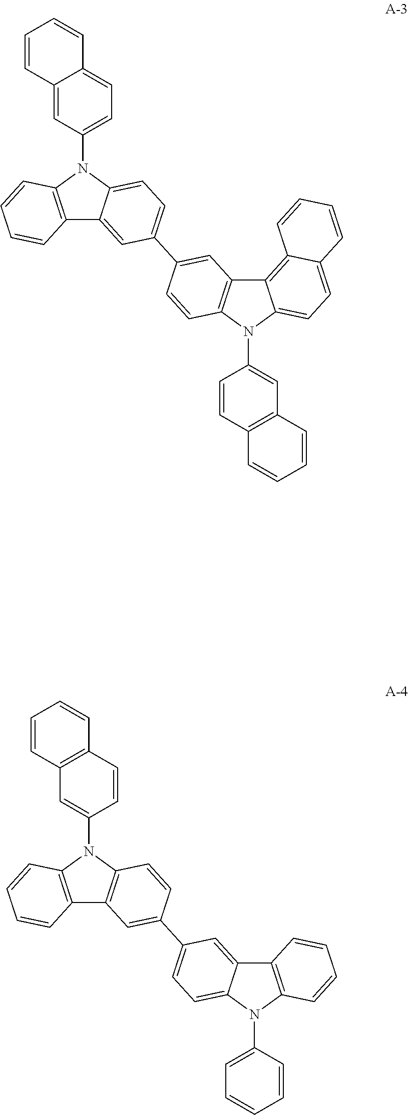 [SMILES: c1ccc2cc(-n3c4ccccc4c4cc(-c5ccc6c(c5)c5c7ccccc7ccc5n6-c5ccc6ccccc6c5)ccc43)ccc2c1 ; c1ccc(-n2c3ccccc3c3cc(-c4ccc5c(c4)c4ccccc4n5-c4ccc5ccccc5c4)ccc32)cc1]

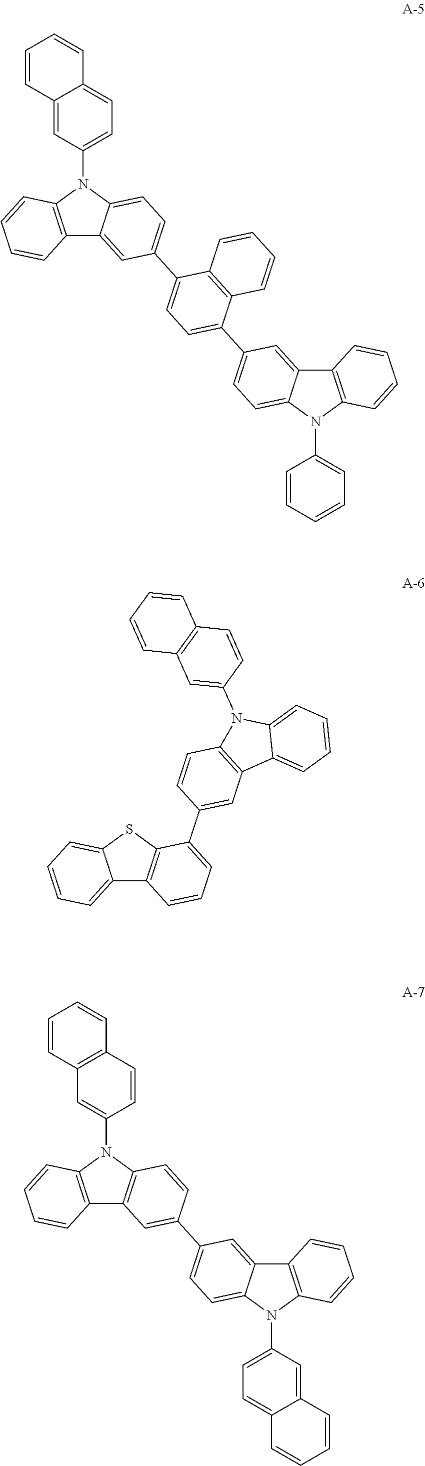 [SMILES: c1ccc(-n2c3ccccc3c3cc(-c4ccc(-c5ccc6c(c5)c5ccccc5n6-c5ccc6ccccc6c5)c5ccccc45)ccc32)cc1 ; c1ccc2cc(-n3c4ccccc4c4cc(-c5cccc6c5sc5ccccc56)ccc43)ccc2c1 ; c1ccc2cc(-n3c4ccccc4c4cc(-c5ccc6c(c5)c5ccccc5n6-c5ccc6ccccc6c5)ccc43)ccc2c1]

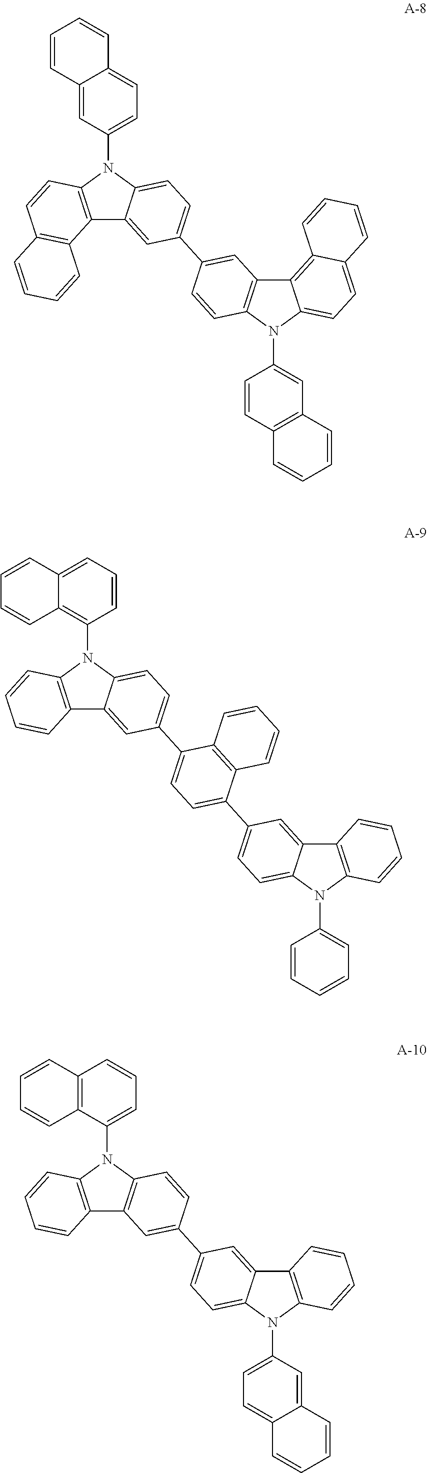 [SMILES: c1ccc2cc(-n3c4ccc(-c5ccc6c(c5)c5c7ccccc7ccc5n6-c5ccc6ccccc6c5)cc4c4c5ccccc5ccc43)ccc2c1 ; c1ccc(-n2c3ccccc3c3cc(-c4ccc(-c5ccc6c(c5)c5ccccc5n6-c5cccc6ccccc56)c5ccccc45)ccc32)cc1 ; c1ccc2cc(-n3c4ccccc4c4cc(-c5ccc6c(c5)c5ccccc5n6-c5cccc6ccccc56)ccc43)ccc2c1]

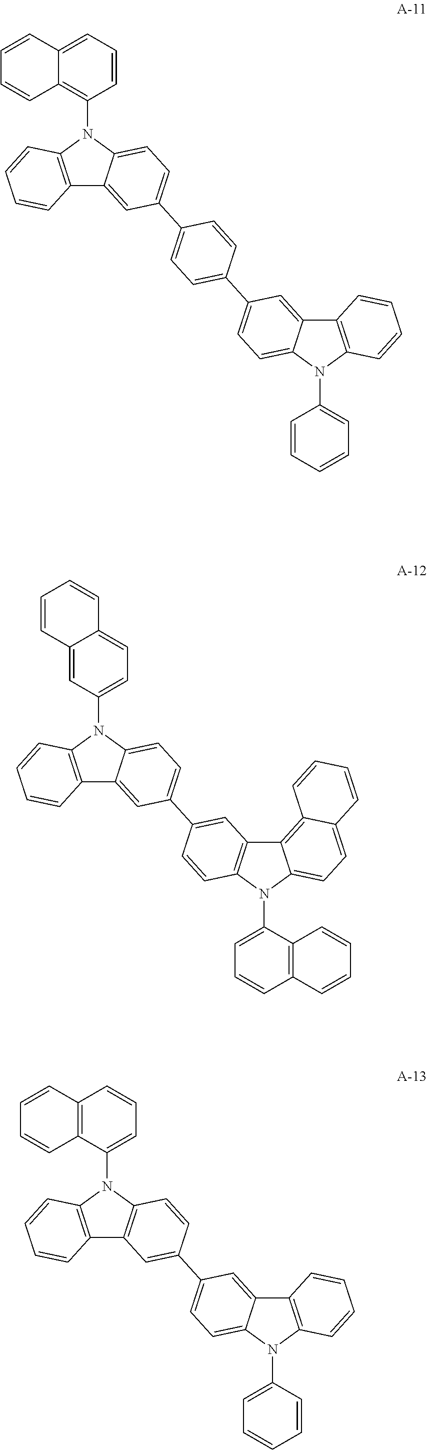 [SMILES: c1ccc(-n2c3ccccc3c3cc(-c4ccc(-c5ccc6c(c5)c5ccccc5n6-c5cccc6ccccc56)cc4)ccc32)cc1 ; c1ccc2cc(-n3c4ccccc4c4cc(-c5ccc6c(c5)c5c7ccccc7ccc5n6-c5cccc6ccccc56)ccc43)ccc2c1 ; c1ccc(-n2c3ccccc3c3cc(-c4ccc5c(c4)c4ccccc4n5-c4cccc5ccccc45)ccc32)cc1]

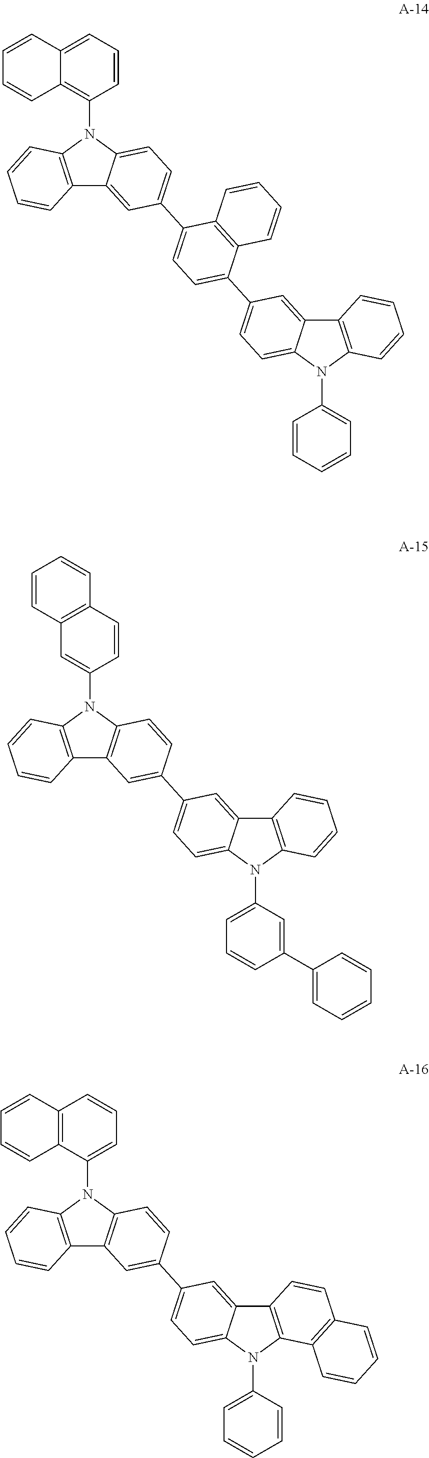 [SMILES: c1ccc(-n2c3ccccc3c3cc(-c4ccc(-c5ccc6c(c5)c5ccccc5n6-c5cccc6ccccc56)c5ccccc45)ccc32)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-n2c3ccc(-c4ccc5c(c4)c4ccccc4n5-c4cccc5ccccc45)cc3c3ccc4ccccc4c32)cc1]

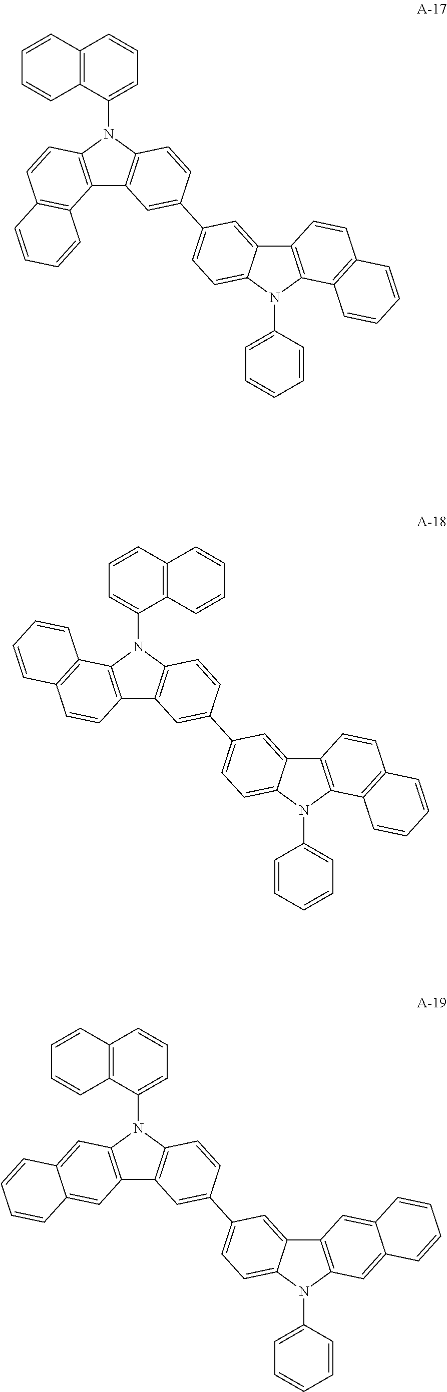 [SMILES: c1ccc(-n2c3ccc(-c4ccc5c(c4)c4c6ccccc6ccc4n5-c4cccc5ccccc45)cc3c3ccc4ccccc4c32)cc1 ; c1ccc(-n2c3ccc(-c4ccc5c(c4)c4ccc6ccccc6c4n5-c4cccc5ccccc45)cc3c3ccc4ccccc4c32)cc1 ; c1ccc(-n2c3ccc(-c4ccc5c(c4)c4cc6ccccc6cc4n5-c4cccc5ccccc45)cc3c3cc4ccccc4cc32)cc1]

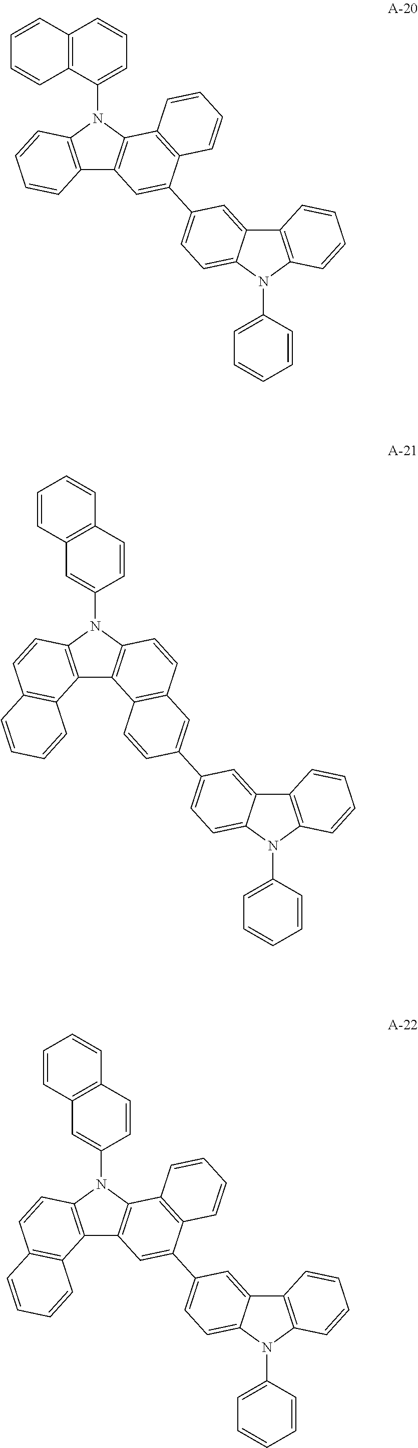 [SMILES: c1ccc(-n2c3ccccc3c3cc(-c4cc5c6ccccc6n(-c6cccc7ccccc67)c5c5ccccc45)ccc32)cc1 ; c1ccc(-n2c3ccccc3c3cc(-c4ccc5c(ccc6c5c5c7ccccc7ccc5n6-c5ccc6ccccc6c5)c4)ccc32)cc1 ; c1ccc(-n2c3ccccc3c3cc(-c4cc5c6c7ccccc7ccc6n(-c6ccc7ccccc7c6)c5c5ccccc45)ccc32)cc1]

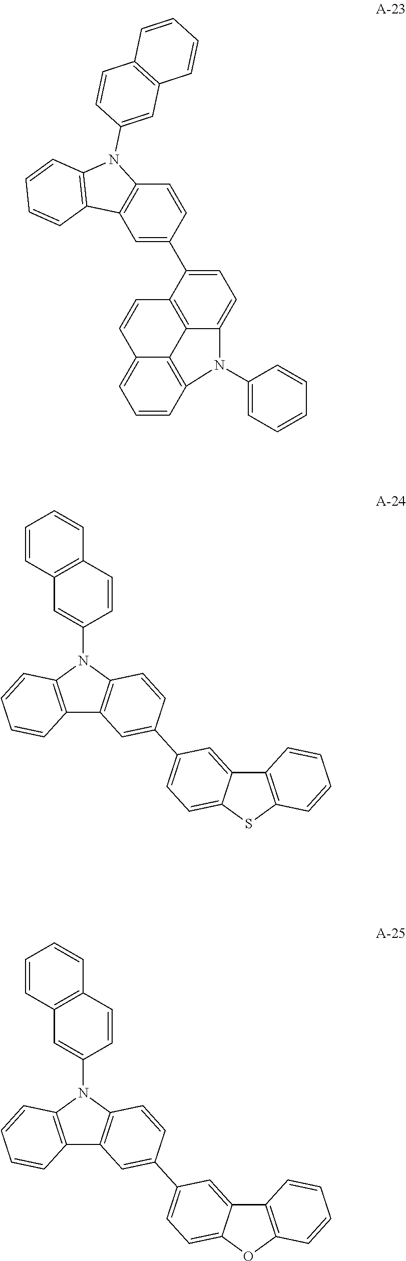 [SMILES: c1ccc(-n2c3cccc4ccc5c(-c6ccc7c(c6)c6ccccc6n7-c6ccc7ccccc7c6)ccc2c5c43)cc1 ; c1ccc2cc(-n3c4ccccc4c4cc(-c5ccc6sc7ccccc7c6c5)ccc43)ccc2c1 ; c1ccc2cc(-n3c4ccccc4c4cc(-c5ccc6oc7ccccc7c6c5)ccc43)ccc2c1]

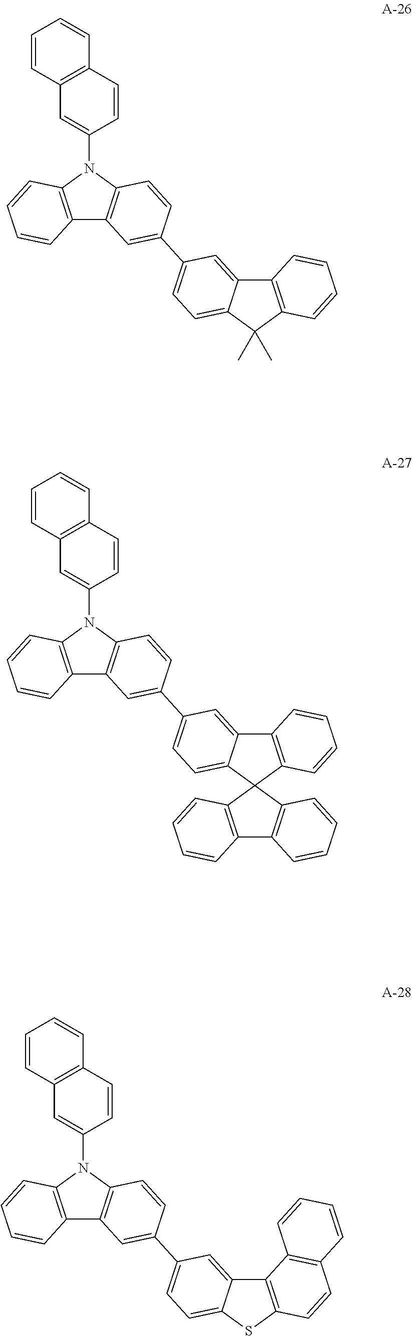 [SMILES: CC1(C)c2ccccc2-c2cc(-c3ccc4c(c3)c3ccccc3n4-c3ccc4ccccc4c3)ccc21 ; c1ccc2c(c1)-c1ccccc1C21c2ccccc2-c2cc(-c3ccc4c(c3)c3ccccc3n4-c3ccc4ccccc4c3)ccc21 ; c1ccc2cc(-n3c4ccccc4c4cc(-c5ccc6sc7ccc8ccccc8c7c6c5)ccc43)ccc2c1]

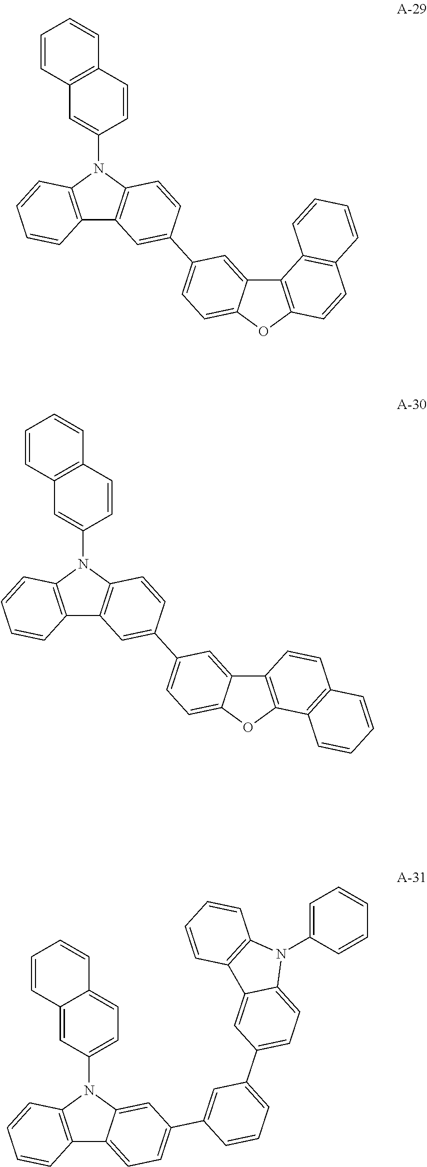 [SMILES: c1ccc2cc(-n3c4ccccc4c4cc(-c5ccc6oc7ccc8ccccc8c7c6c5)ccc43)ccc2c1 ; c1ccc2cc(-n3c4ccccc4c4cc(-c5ccc6oc7c8ccccc8ccc7c6c5)ccc43)ccc2c1 ; c1ccc(-n2c3ccccc3c3cc(-c4cccc(-c5ccc6c7ccccc7n(-c7ccc8ccccc8c7)c6c5)c4)ccc32)cc1]

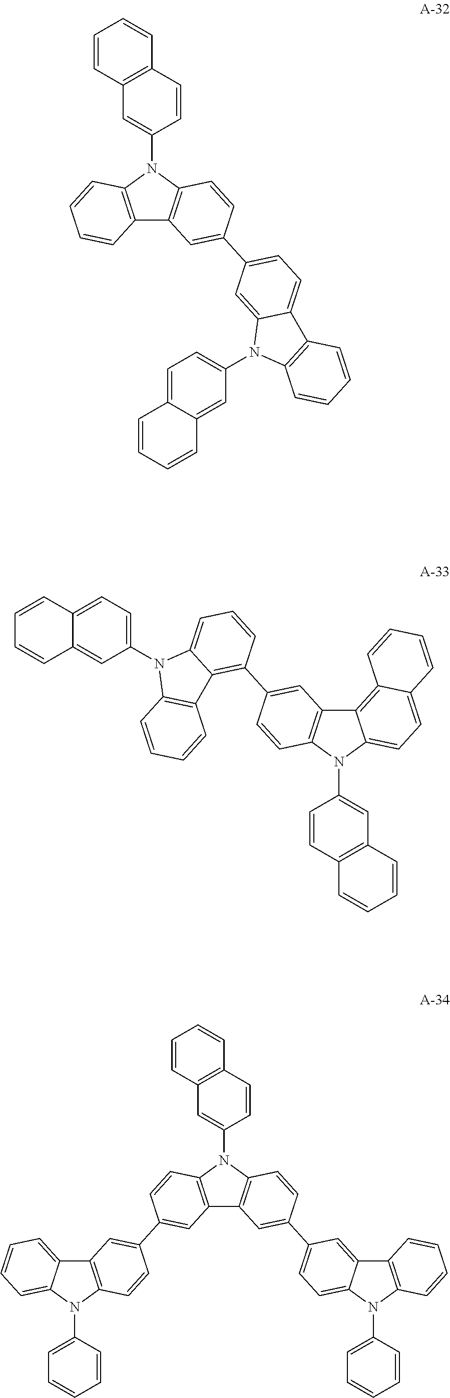 [SMILES: c1ccc2cc(-n3c4ccccc4c4cc(-c5ccc6c7ccccc7n(-c7ccc8ccccc8c7)c6c5)ccc43)ccc2c1 ; c1ccc2cc(-n3c4ccccc4c4c(-c5ccc6c(c5)c5c7ccccc7ccc5n6-c5ccc6ccccc6c5)cccc43)ccc2c1 ; c1ccc(-n2c3ccccc3c3cc(-c4ccc5c(c4)c4cc(-c6ccc7c(c6)c6ccccc6n7-c6ccccc6)ccc4n5-c4ccc5ccccc5c4)ccc32)cc1]

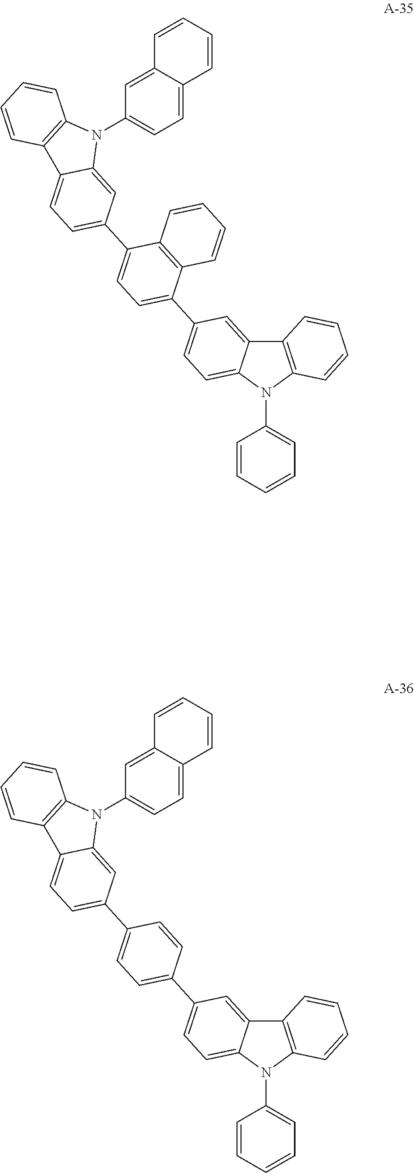 [SMILES: c1ccc(-n2c3ccccc3c3cc(-c4ccc(-c5ccc6c7ccccc7n(-c7ccc8ccccc8c7)c6c5)c5ccccc45)ccc32)cc1 ; c1ccc(-n2c3ccccc3c3cc(-c4ccc(-c5ccc6c7ccccc7n(-c7ccc8ccccc8c7)c6c5)cc4)ccc32)cc1]

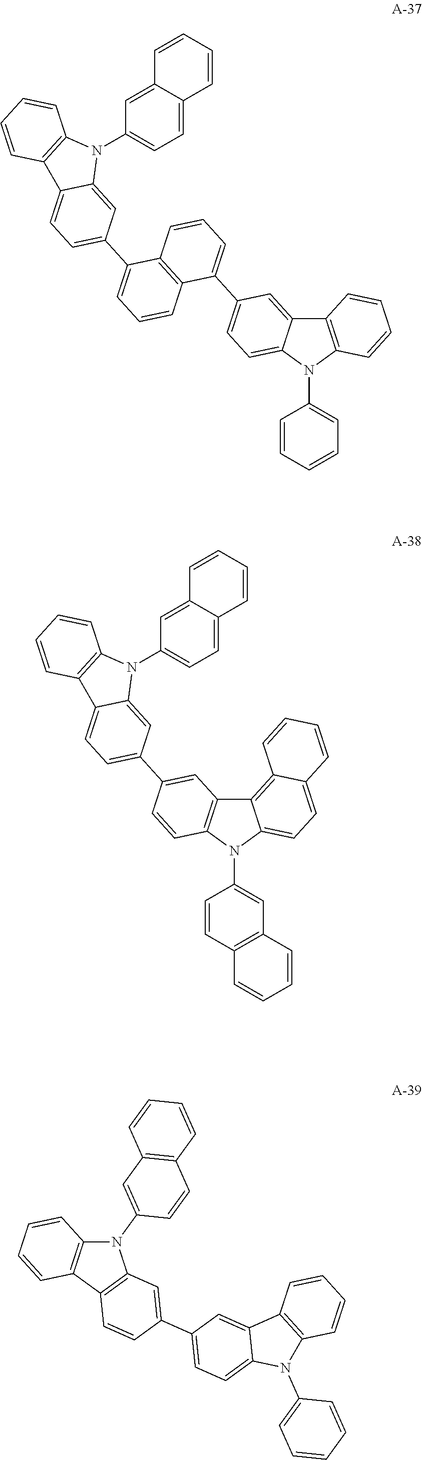 [SMILES: c1ccc(-n2c3ccccc3c3cc(-c4cccc5c(-c6ccc7c8ccccc8n(-c8ccc9ccccc9c8)c7c6)cccc45)ccc32)cc1 ; c1ccc2cc(-n3c4ccccc4c4ccc(-c5ccc6c(c5)c5c7ccccc7ccc5n6-c5ccc6ccccc6c5)cc43)ccc2c1 ; c1ccc(-n2c3ccccc3c3cc(-c4ccc5c6ccccc6n(-c6ccc7ccccc7c6)c5c4)ccc32)cc1]

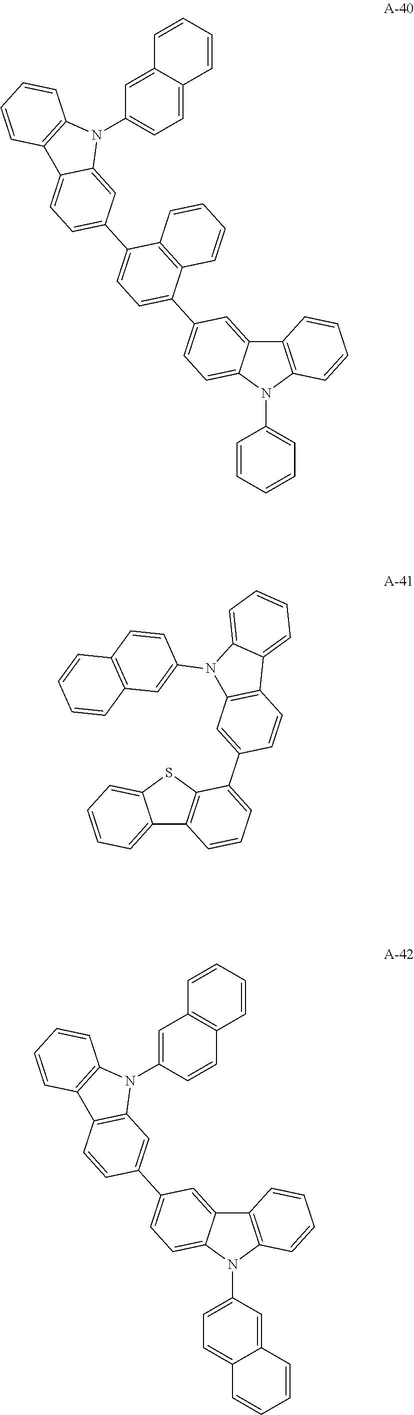 [SMILES: c1ccc(-n2c3ccccc3c3cc(-c4ccc(-c5ccc6c7ccccc7n(-c7ccc8ccccc8c7)c6c5)c5ccccc45)ccc32)cc1 ; c1ccc2cc(-n3c4ccccc4c4ccc(-c5cccc6c5sc5ccccc56)cc43)ccc2c1 ; c1ccc2cc(-n3c4ccccc4c4cc(-c5ccc6c7ccccc7n(-c7ccc8ccccc8c7)c6c5)ccc43)ccc2c1]

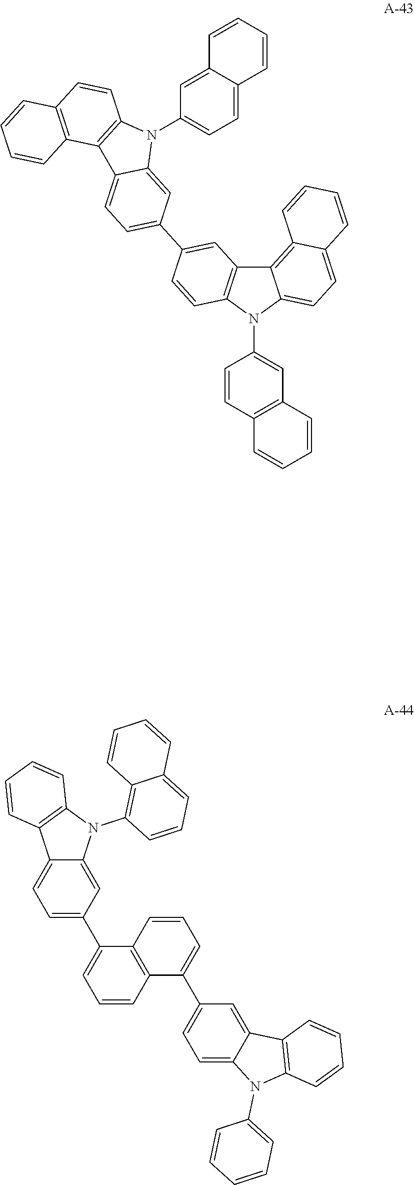 [SMILES: c1ccc2cc(-n3c4ccc(-c5ccc6c7c8ccccc8ccc7n(-c7ccc8ccccc8c7)c6c5)cc4c4c5ccccc5ccc43)ccc2c1 ; c1ccc(-n2c3ccccc3c3cc(-c4cccc5c(-c6ccc7c8ccccc8n(-c8cccc9ccccc89)c7c6)cccc45)ccc32)cc1]

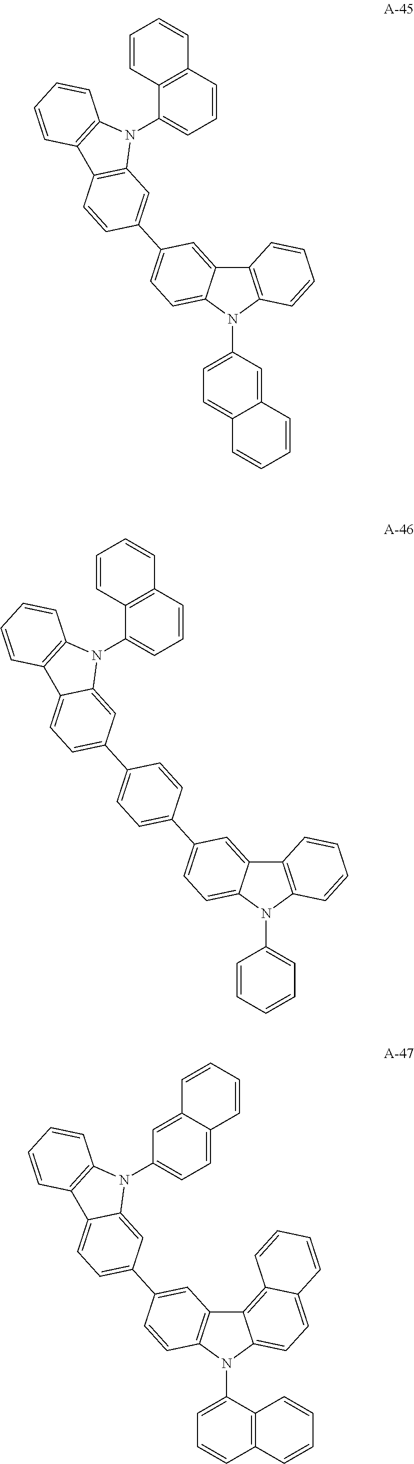 [SMILES: c1ccc2cc(-n3c4ccccc4c4cc(-c5ccc6c7ccccc7n(-c7cccc8ccccc78)c6c5)ccc43)ccc2c1 ; c1ccc(-n2c3ccccc3c3cc(-c4ccc(-c5ccc6c7ccccc7n(-c7cccc8ccccc78)c6c5)cc4)ccc32)cc1 ; c1ccc2cc(-n3c4ccccc4c4ccc(-c5ccc6c(c5)c5c7ccccc7ccc5n6-c5cccc6ccccc56)cc43)ccc2c1]

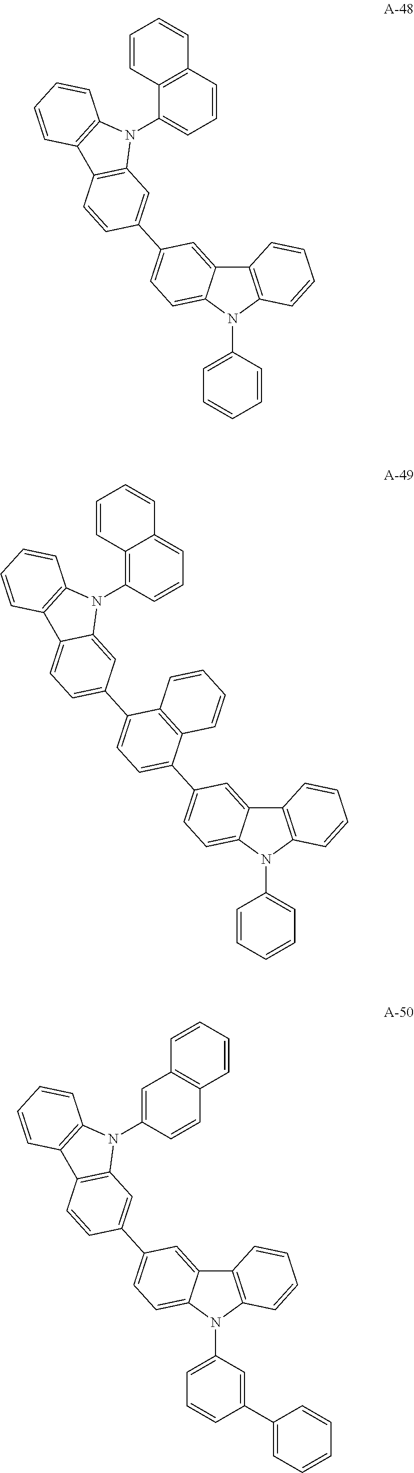 [SMILES: c1ccc(-n2c3ccccc3c3cc(-c4ccc5c6ccccc6n(-c6cccc7ccccc67)c5c4)ccc32)cc1 ; c1ccc(-n2c3ccccc3c3cc(-c4ccc(-c5ccc6c7ccccc7n(-c7cccc8ccccc78)c6c5)c5ccccc45)ccc32)cc1 ; c1ccc(-c2cccc(-n3c4ccccc4c4cc(-c5ccc6c7ccccc7n(-c7ccc8ccccc8c7)c6c5)ccc43)c2)cc1]

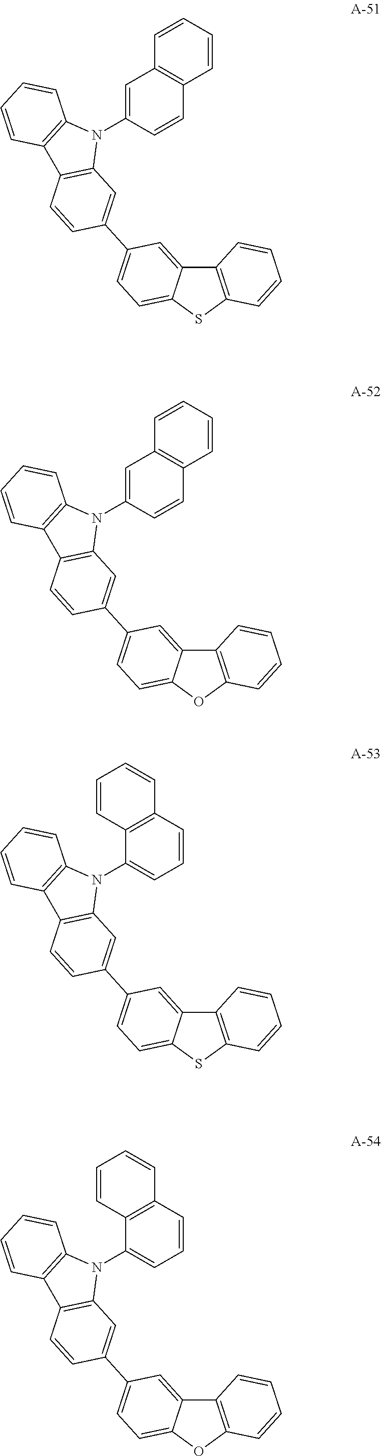 [SMILES: c1ccc2cc(-n3c4ccccc4c4ccc(-c5ccc6sc7ccccc7c6c5)cc43)ccc2c1 ; c1ccc2cc(-n3c4ccccc4c4ccc(-c5ccc6oc7ccccc7c6c5)cc43)ccc2c1 ; c1ccc2c(-n3c4ccccc4c4ccc(-c5ccc6sc7ccccc7c6c5)cc43)cccc2c1 ; c1ccc2c(-n3c4ccccc4c4ccc(-c5ccc6oc7ccccc7c6c5)cc43)cccc2c1]

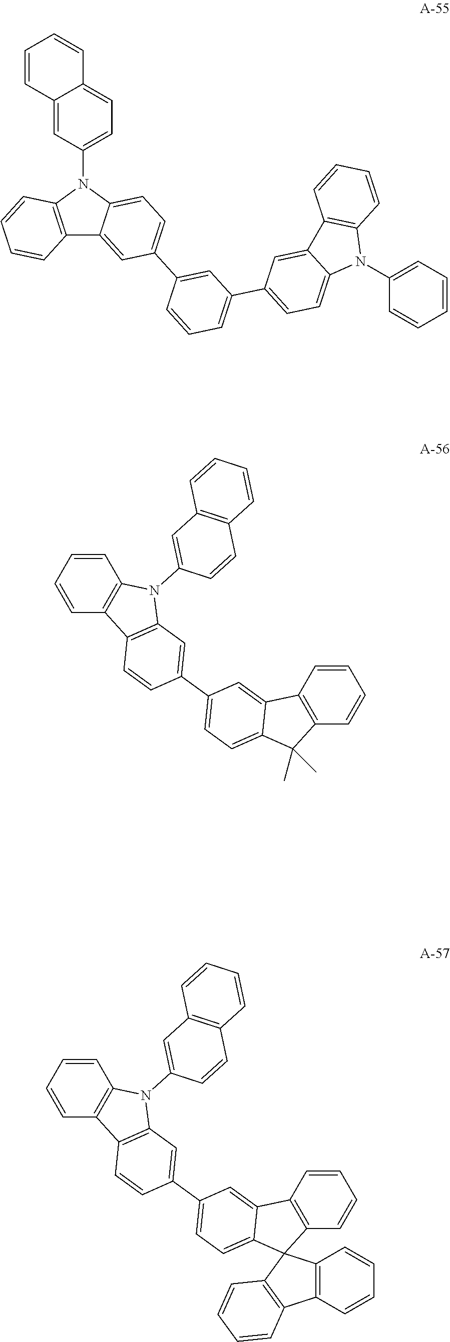 [SMILES: c1ccc(-n2c3ccccc3c3cc(-c4cccc(-c5ccc6c(c5)c5ccccc5n6-c5ccc6ccccc6c5)c4)ccc32)cc1 ; CC1(C)c2ccccc2-c2cc(-c3ccc4c5ccccc5n(-c5ccc6ccccc6c5)c4c3)ccc21 ; c1ccc2c(c1)-c1ccccc1C21c2ccccc2-c2cc(-c3ccc4c5ccccc5n(-c5ccc6ccccc6c5)c4c3)ccc21]

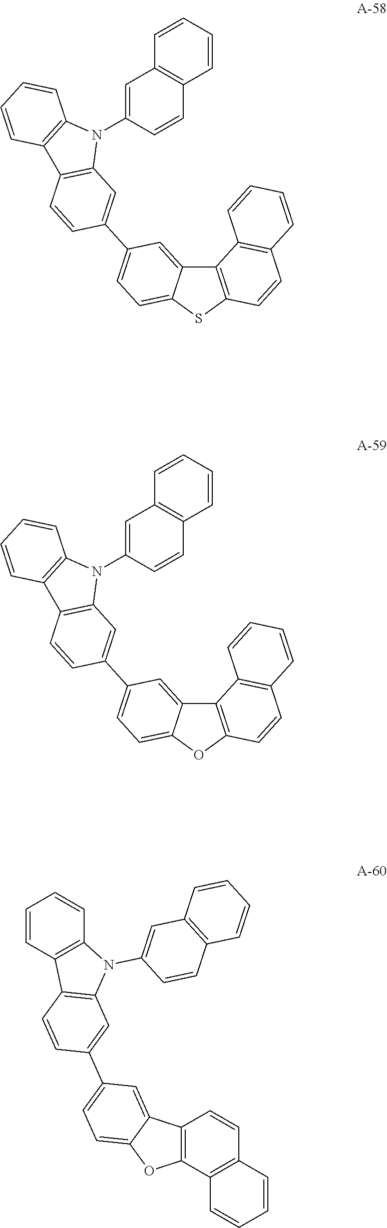 [SMILES: c1ccc2cc(-n3c4ccccc4c4ccc(-c5ccc6sc7ccc8ccccc8c7c6c5)cc43)ccc2c1 ; c1ccc2cc(-n3c4ccccc4c4ccc(-c5ccc6oc7ccc8ccccc8c7c6c5)cc43)ccc2c1 ; c1ccc2cc(-n3c4ccccc4c4ccc(-c5ccc6oc7c8ccccc8ccc7c6c5)cc43)ccc2c1]

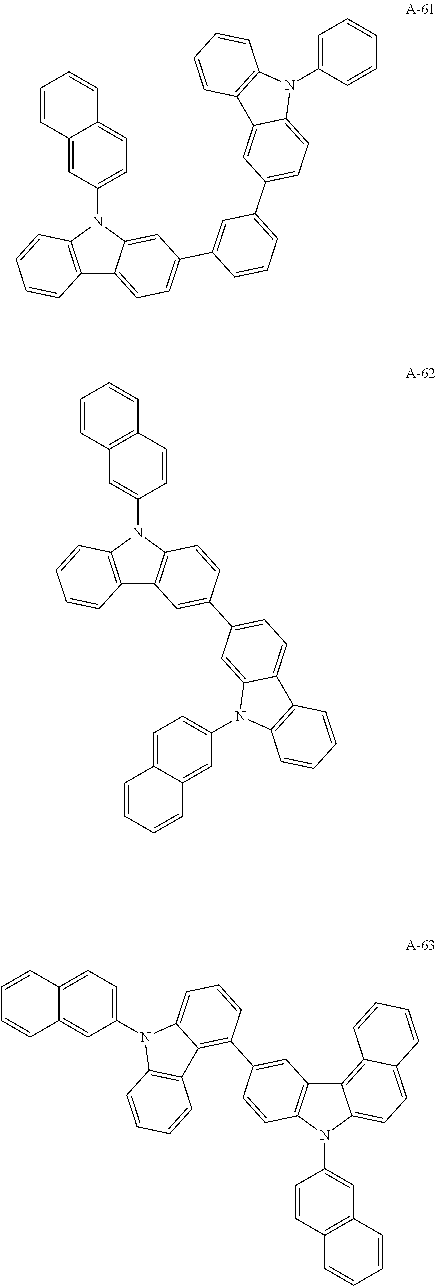 [SMILES: c1ccc(-n2c3ccccc3c3cc(-c4cccc(-c5ccc6c7ccccc7n(-c7ccc8ccccc8c7)c6c5)c4)ccc32)cc1 ; c1ccc2cc(-n3c4ccccc4c4cc(-c5ccc6c7ccccc7n(-c7ccc8ccccc8c7)c6c5)ccc43)ccc2c1 ; c1ccc2cc(-n3c4ccccc4c4c(-c5ccc6c(c5)c5c7ccccc7ccc5n6-c5ccc6ccccc6c5)cccc43)ccc2c1]

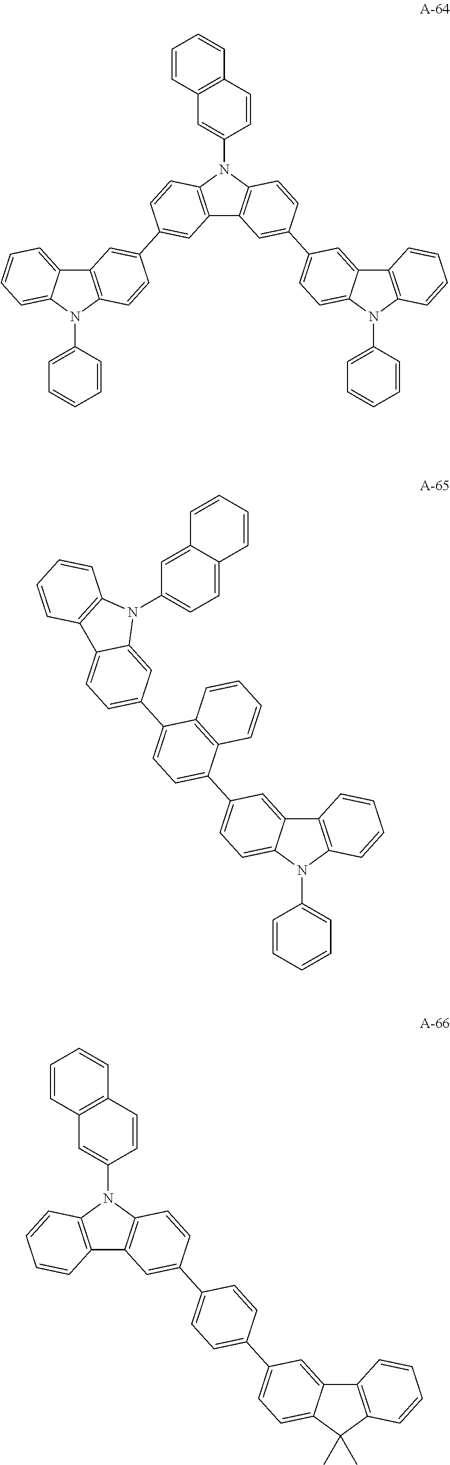 [SMILES: c1ccc(-n2c3ccccc3c3cc(-c4ccc5c(c4)c4cc(-c6ccc7c(c6)c6ccccc6n7-c6ccccc6)ccc4n5-c4ccc5ccccc5c4)ccc32)cc1 ; c1ccc(-n2c3ccccc3c3cc(-c4ccc(-c5ccc6c7ccccc7n(-c7ccc8ccccc8c7)c6c5)c5ccccc45)ccc32)cc1 ; CC1(C)c2ccccc2-c2cc(-c3ccc(-c4ccc5c(c4)c4ccccc4n5-c4ccc5ccccc5c4)cc3)ccc21]

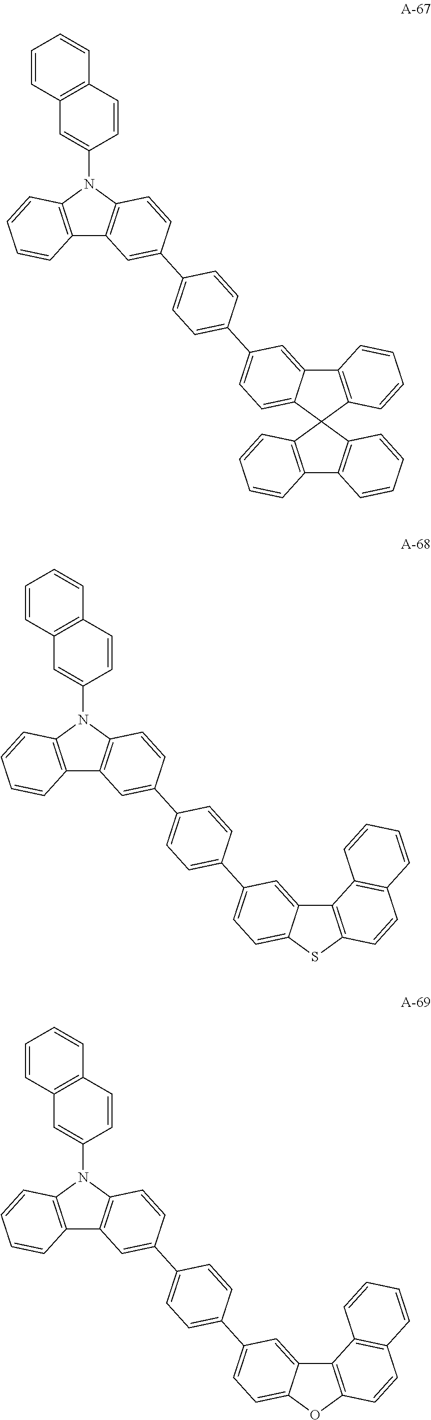 [SMILES: c1ccc2c(c1)-c1ccccc1C21c2ccccc2-c2cc(-c3ccc(-c4ccc5c(c4)c4ccccc4n5-c4ccc5ccccc5c4)cc3)ccc21 ; c1ccc2cc(-n3c4ccccc4c4cc(-c5ccc(-c6ccc7sc8ccc9ccccc9c8c7c6)cc5)ccc43)ccc2c1 ; c1ccc2cc(-n3c4ccccc4c4cc(-c5ccc(-c6ccc7oc8ccc9ccccc9c8c7c6)cc5)ccc43)ccc2c1]

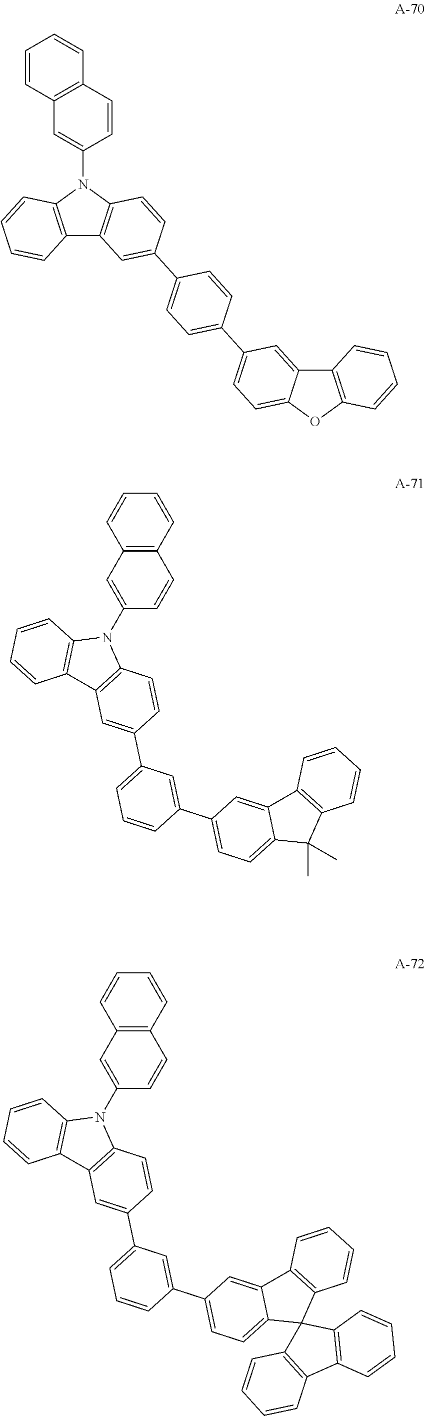 [SMILES: c1ccc2cc(-n3c4ccccc4c4cc(-c5ccc(-c6ccc7oc8ccccc8c7c6)cc5)ccc43)ccc2c1 ; CC1(C)c2ccccc2-c2cc(-c3cccc(-c4ccc5c(c4)c4ccccc4n5-c4ccc5ccccc5c4)c3)ccc21 ; c1cc(-c2ccc3c(c2)-c2ccccc2C32c3ccccc3-c3ccccc32)cc(-c2ccc3c(c2)c2ccccc2n3-c2ccc3ccccc3c2)c1]

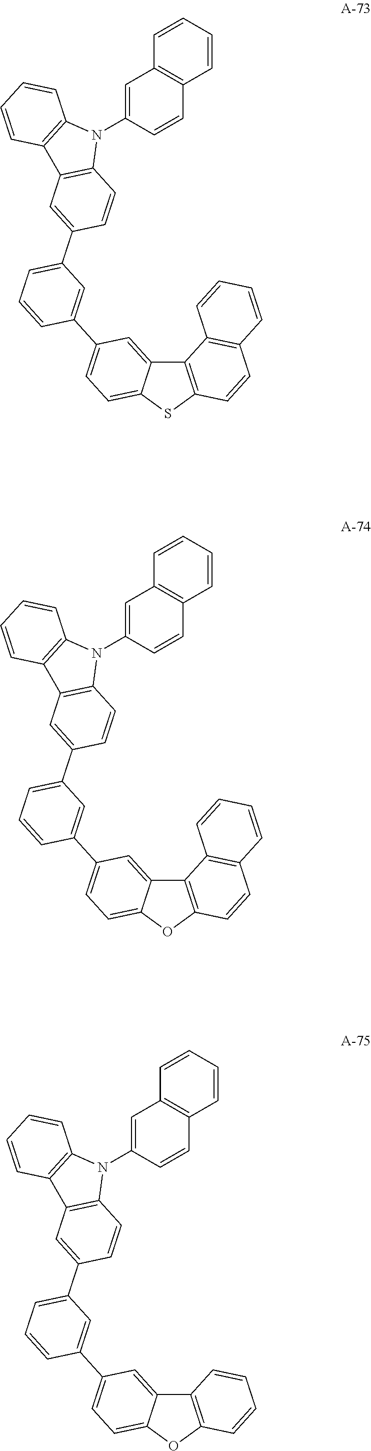 [SMILES: c1cc(-c2ccc3sc4ccc5ccccc5c4c3c2)cc(-c2ccc3c(c2)c2ccccc2n3-c2ccc3ccccc3c2)c1 ; c1cc(-c2ccc3oc4ccc5ccccc5c4c3c2)cc(-c2ccc3c(c2)c2ccccc2n3-c2ccc3ccccc3c2)c1 ; c1cc(-c2ccc3oc4ccccc4c3c2)cc(-c2ccc3c(c2)c2ccccc2n3-c2ccc3ccccc3c2)c1]

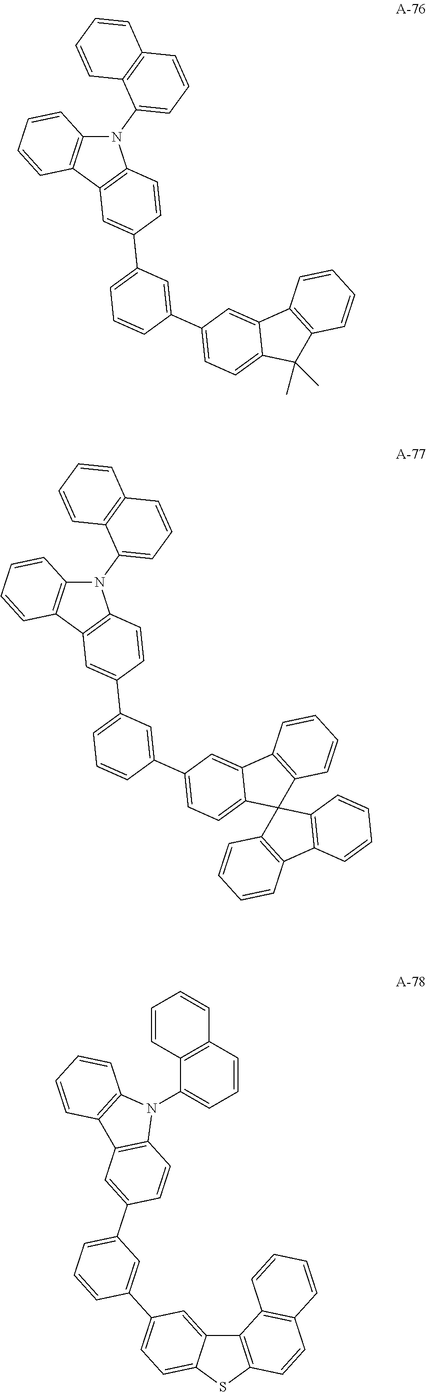 [SMILES: CC1(C)c2ccccc2-c2cc(-c3cccc(-c4ccc5c(c4)c4ccccc4n5-c4cccc5ccccc45)c3)ccc21 ; c1cc(-c2ccc3c(c2)-c2ccccc2C32c3ccccc3-c3ccccc32)cc(-c2ccc3c(c2)c2ccccc2n3-c2cccc3ccccc23)c1 ; c1cc(-c2ccc3sc4ccc5ccccc5c4c3c2)cc(-c2ccc3c(c2)c2ccccc2n3-c2cccc3ccccc23)c1]

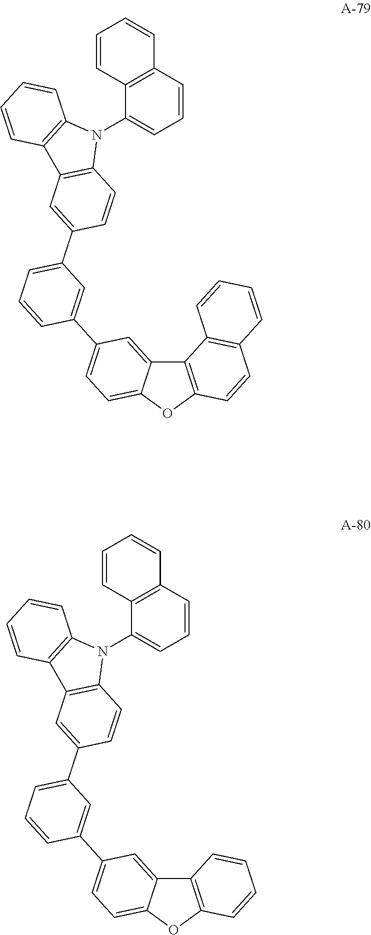 [SMILES: c1cc(-c2ccc3oc4ccc5ccccc5c4c3c2)cc(-c2ccc3c(c2)c2ccccc2n3-c2cccc3ccccc23)c1 ; c1cc(-c2ccc3oc4ccccc4c3c2)cc(-c2ccc3c(c2)c2ccccc2n3-c2cccc3ccccc23)c1]

The compound of formula 1 according to the present invention can be prepared by a synthetic method known to a person skilled in the art.

Another embodiment of the present invention provides the use of the compound represented by formula 1 as a hole transport material. Preferably, the use may be one as a hole transport material of an organic electroluminescent device.

The organic electroluminescent device comprises a first electrode; a second electrode; and at least one organic layer between the first and second electrodes. The organic layer may comprise at least one organic electroluminescent compound of formula 1.

One of the first and second electrodes can be an anode, and the other can be a cathode. The organic layer comprises a light-emitting layer and a hole transport layer, and may further comprise at least one layer selected from the group consisting of a hole injection layer, an electron transport layer, an electron injection layer, an interlayer, a hole blocking layer, and an electron blocking layer.

The compound of formula 1 according to the present invention can be comprised in the hole transport layer. In this case, the compound of formula 1 according to the present invention can be comprised as a hole transport material.

The organic electroluminescent device comprising the compound of formula 1 according to the present invention can further comprise one or more host compounds, and can further comprise one or more dopants.

The host material can be from any of known fluorescent hosts. A compound represented by formula 11 below can be used.

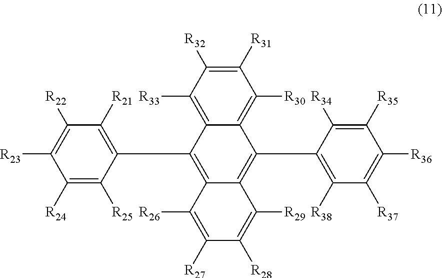

wherein Cz represents the following structure;

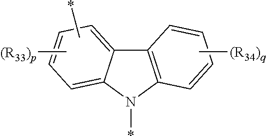

R₂₁ to R₃₅ each independently represent hydrogen, deuterium, a halogen, a cyano, a substituted or unsubstituted (C1-C30)alkyl, a substituted of unsubstituted (C6-C30)aryl, a substituted or unsubstituted 5- to 30-membered heteroaryl, a substituted of unsubstituted (C3-C30)cycloalkyl, a substituted of unsubstituted (C1-C30)alkoxy, a substituted or unsubstituted (C1-C30)alkylsilyl, a substituted of unsubstituted (C6-C30)arylsilyl, or a substituted of unsubstituted (C6-C30)aryl(C1-C30)alkylsilyl; or are linked to each other to form a mono- or polycyclic, (C3-C30) alicyclic or aromatic ring, whose carbon atom(s) may be replaced with at least one hetero atom selected from nitrogen, oxygen, and sulfur.

Specifically, preferable examples of the host material are as follows:

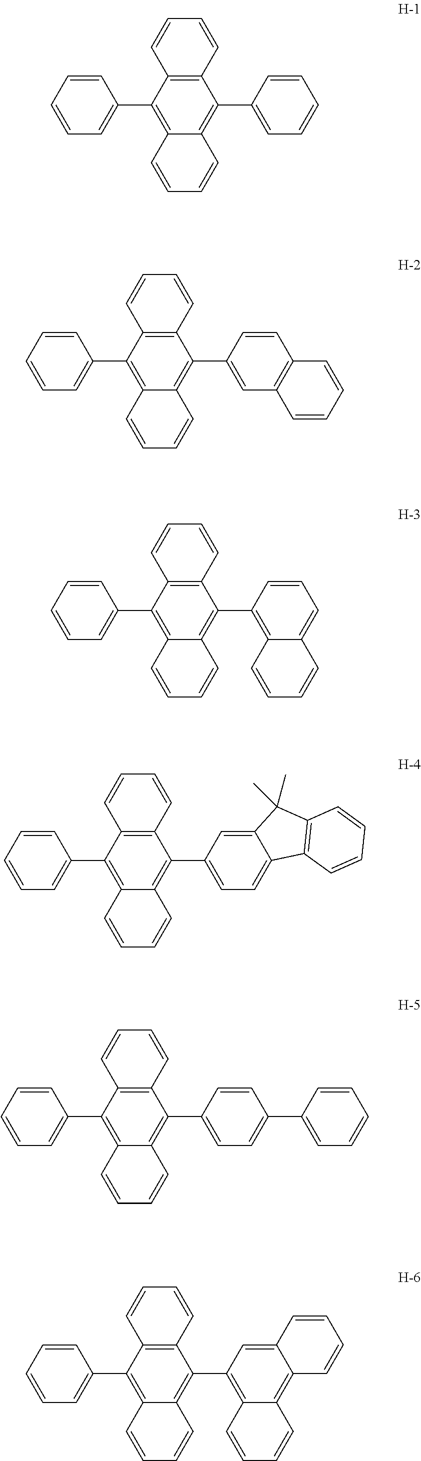

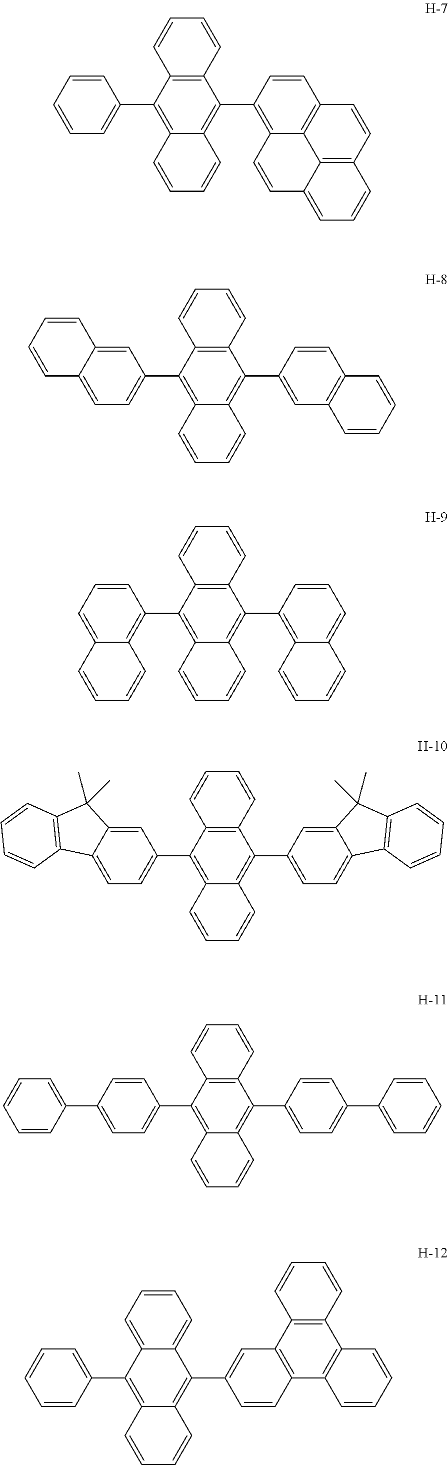

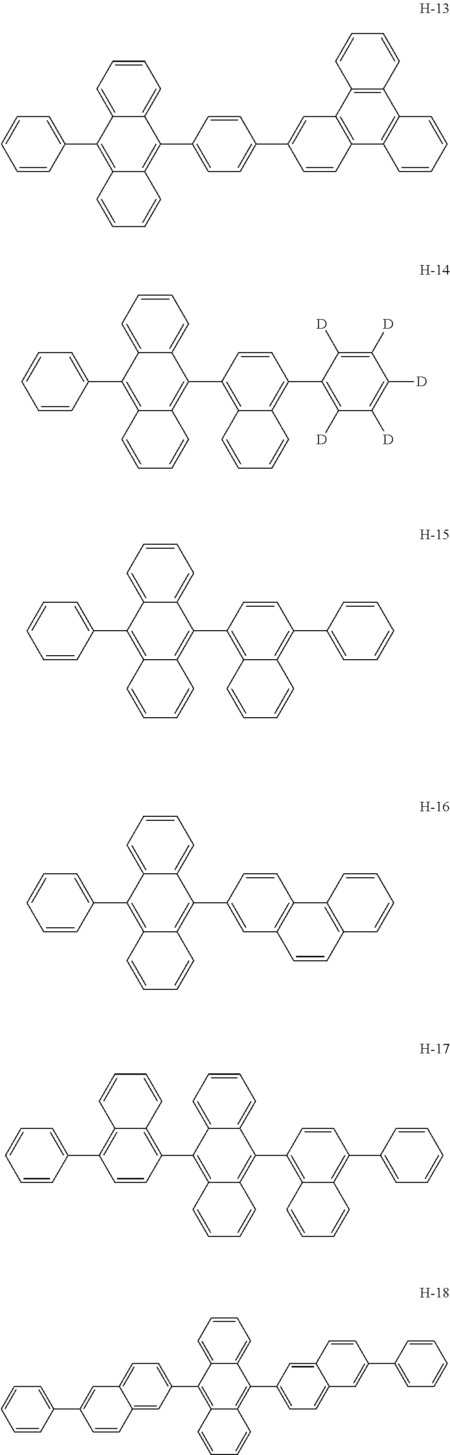

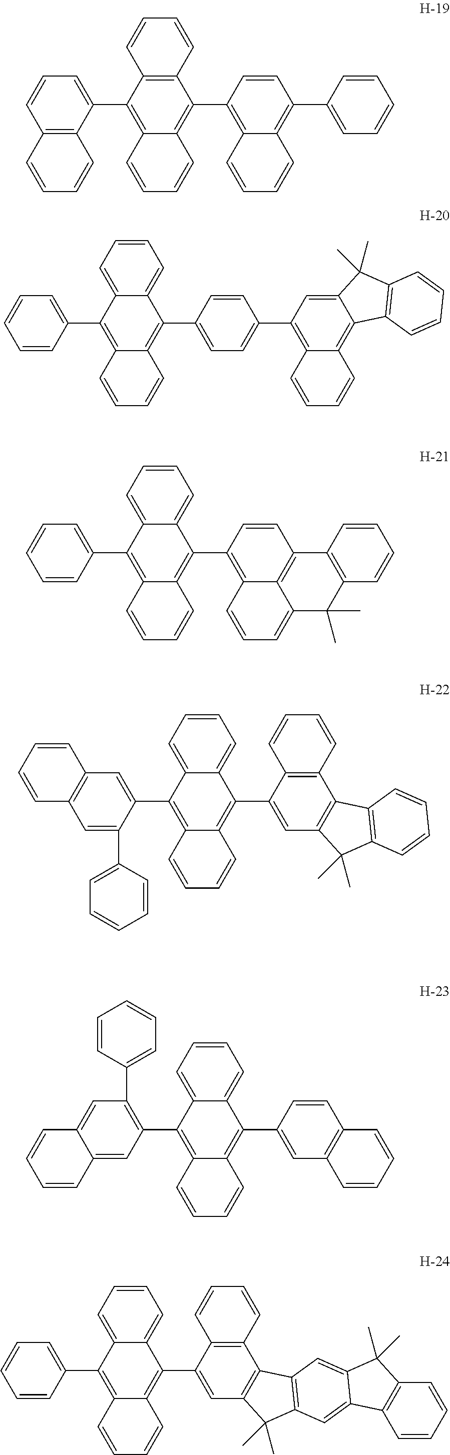

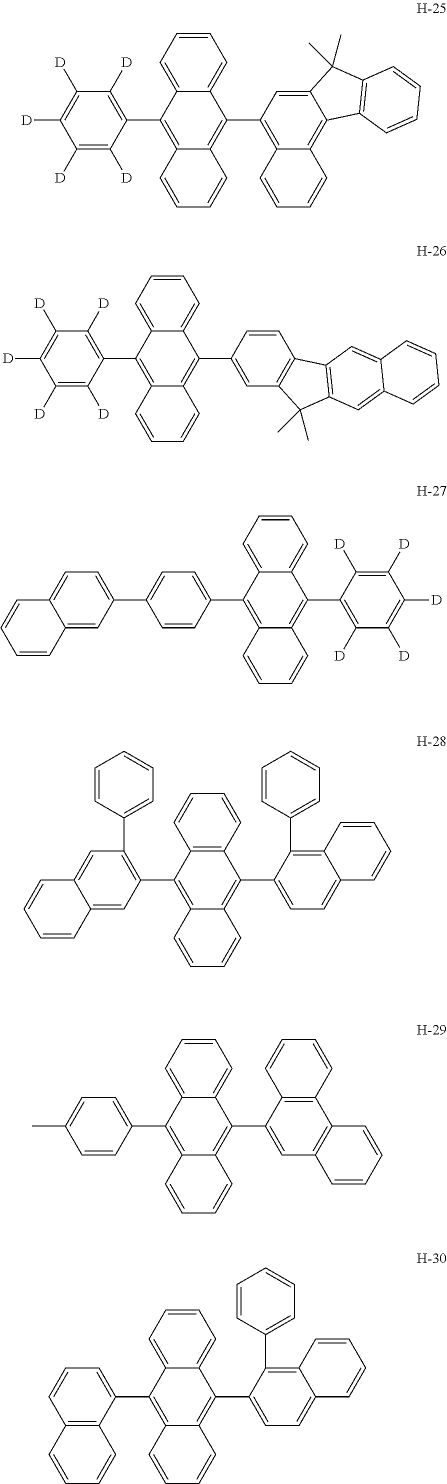

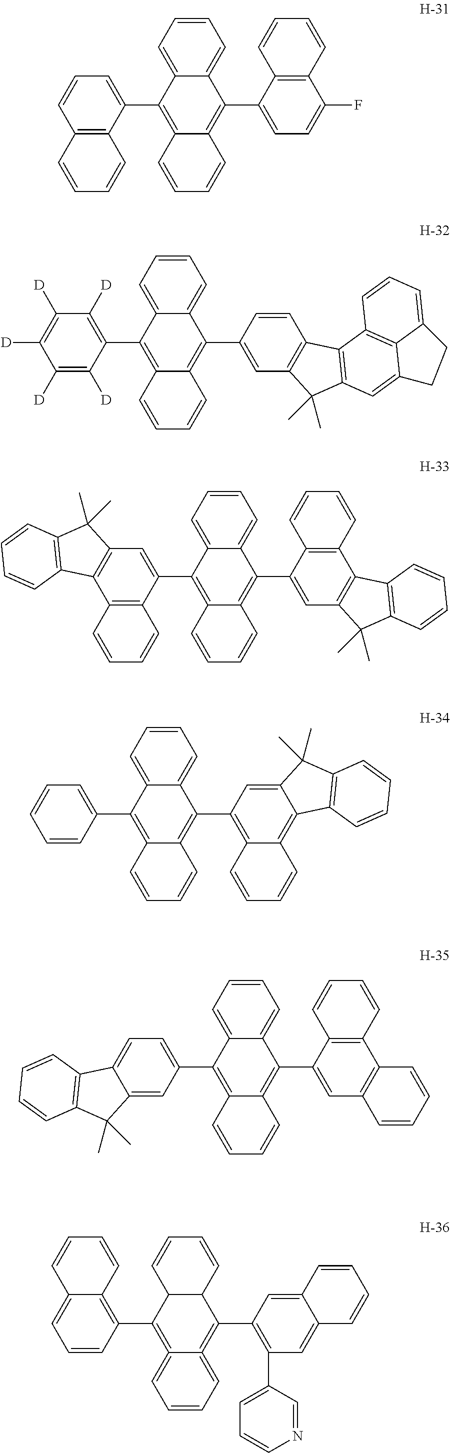

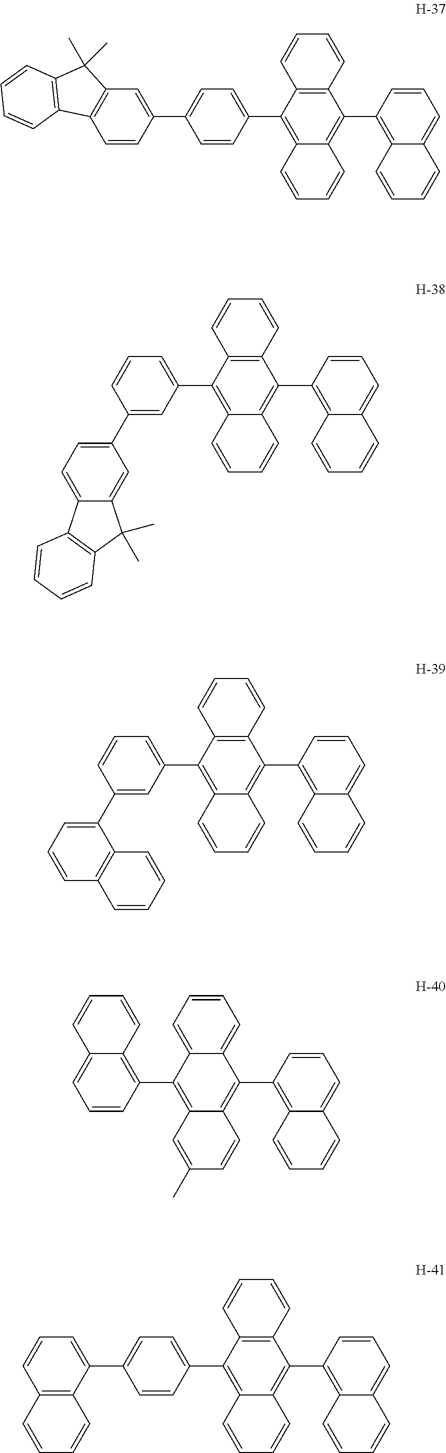

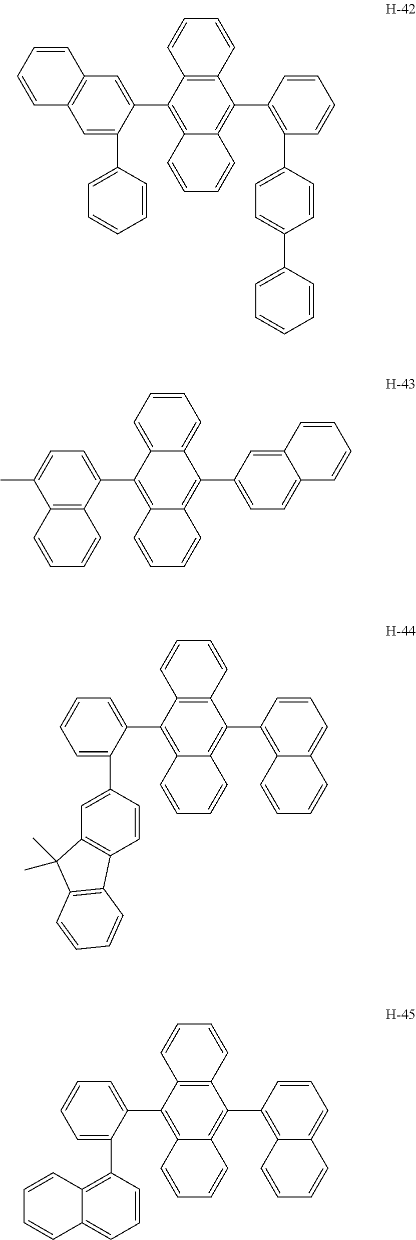

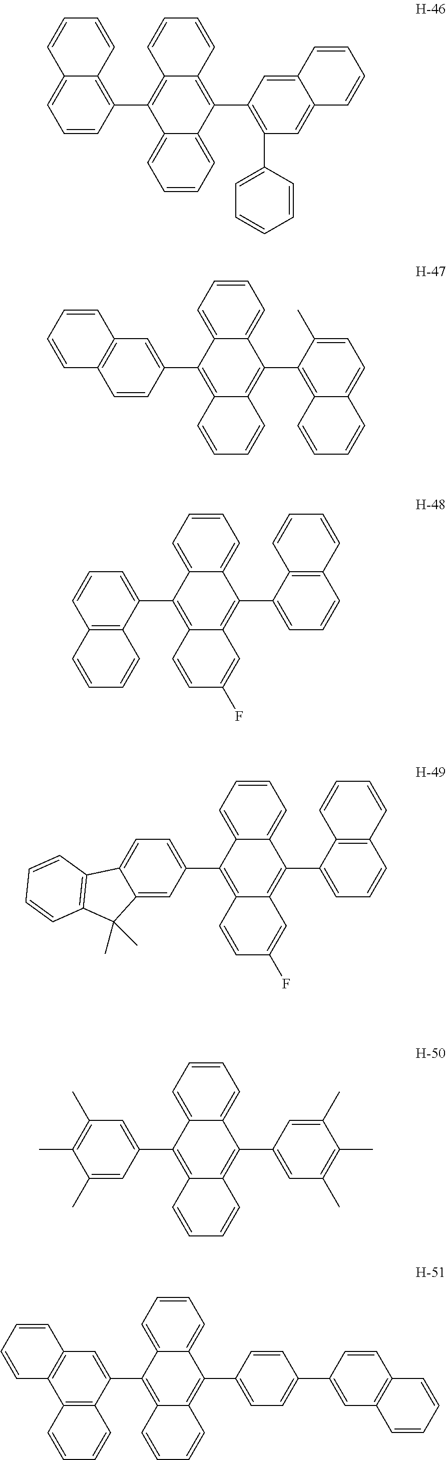

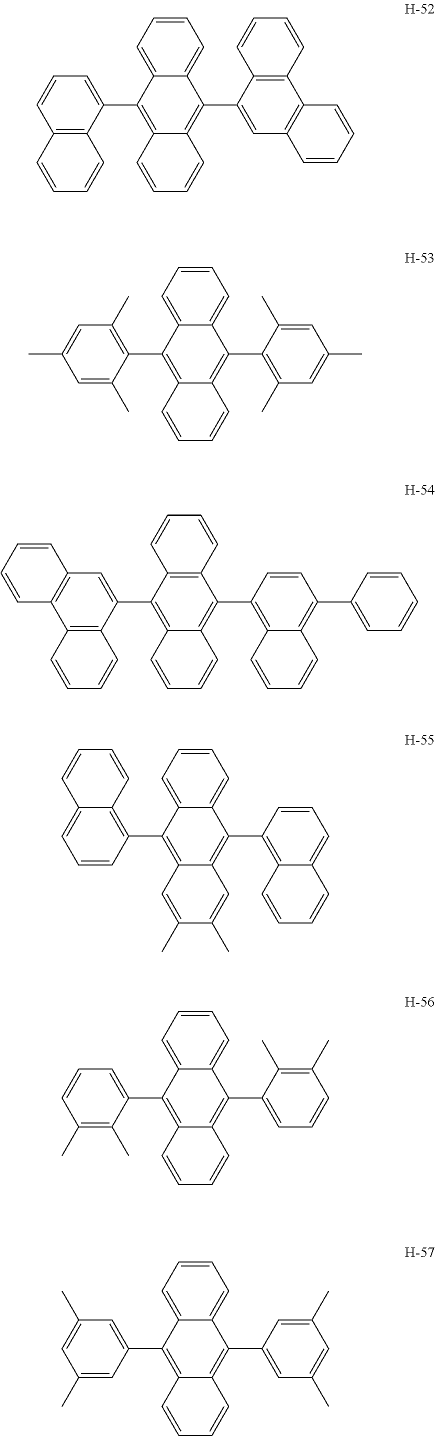

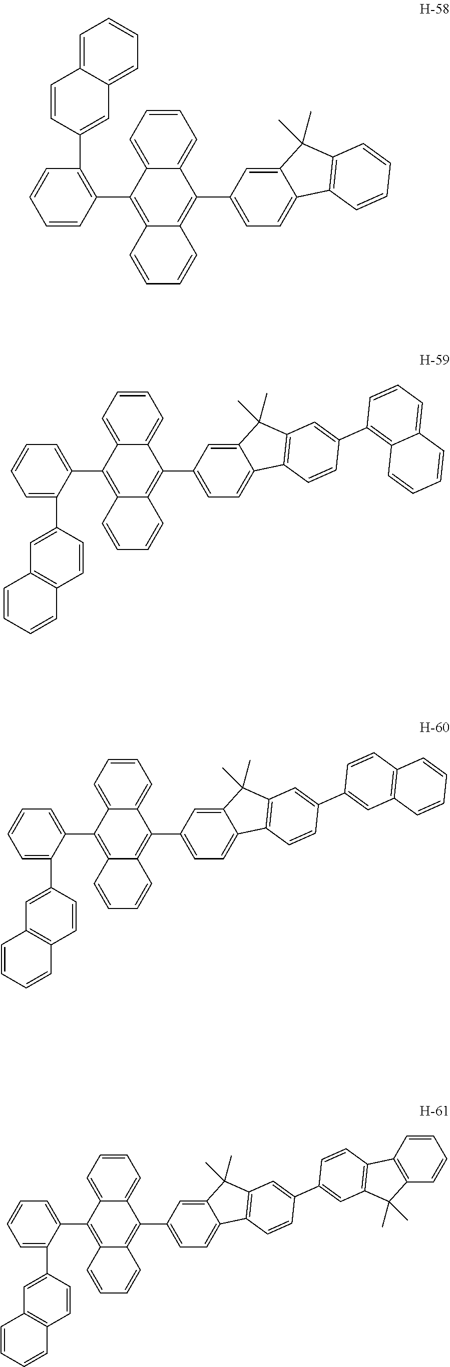

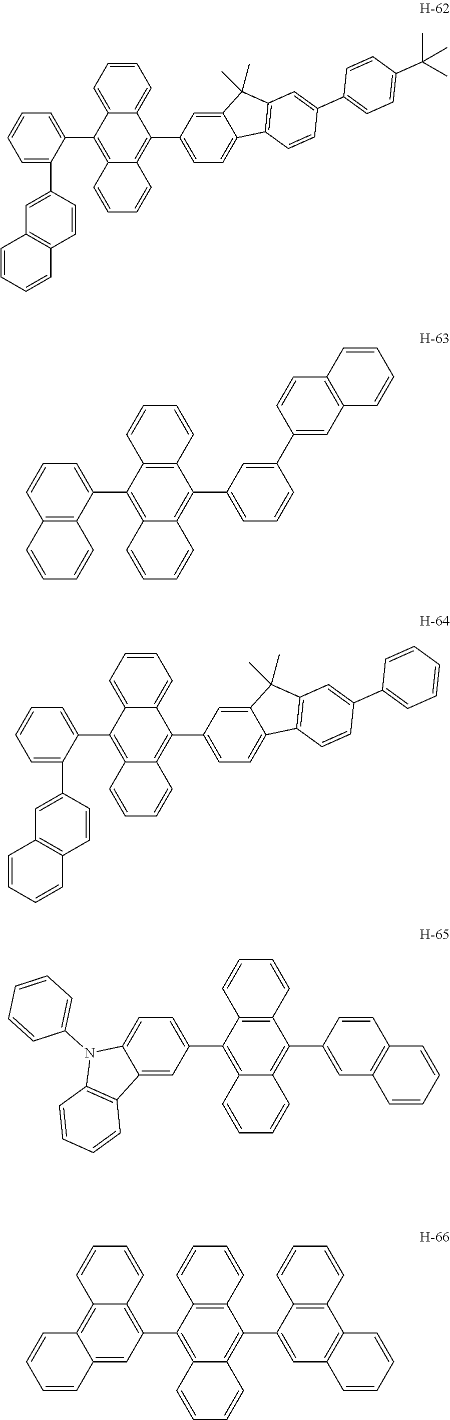

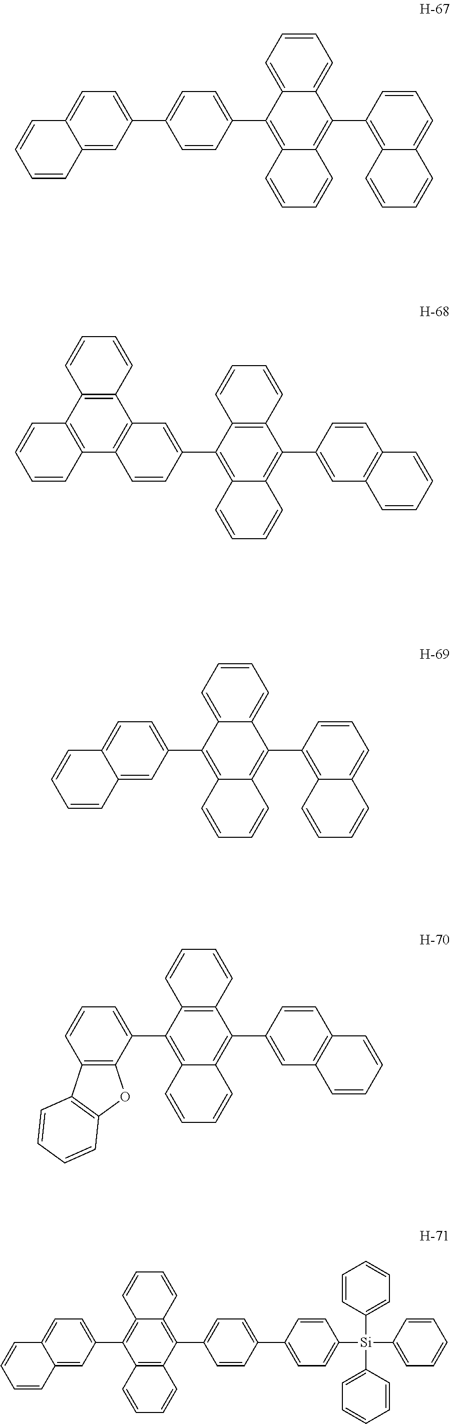

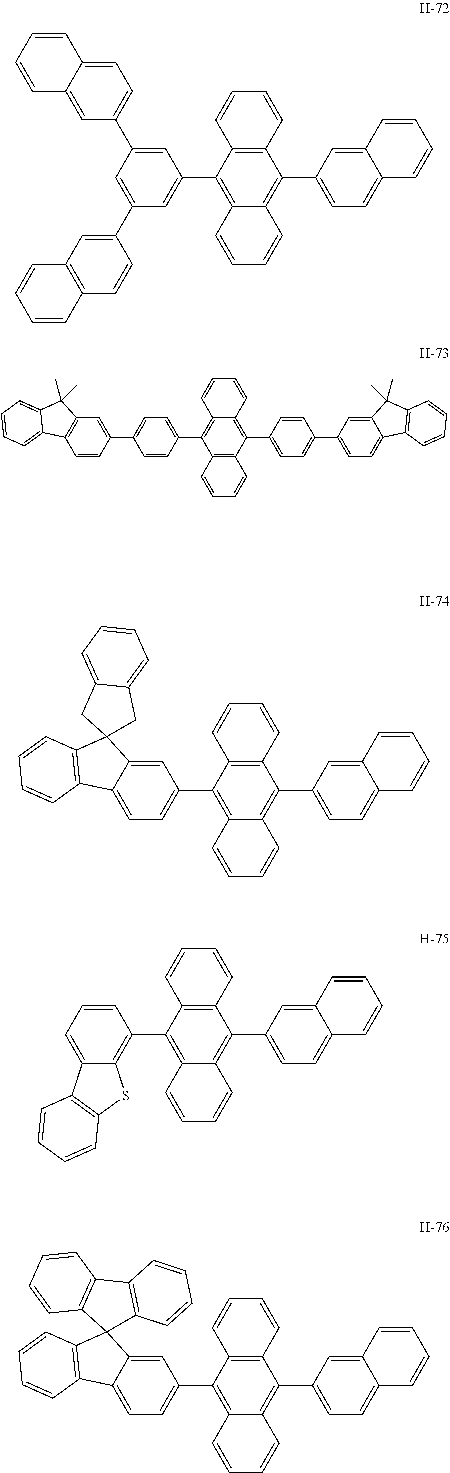

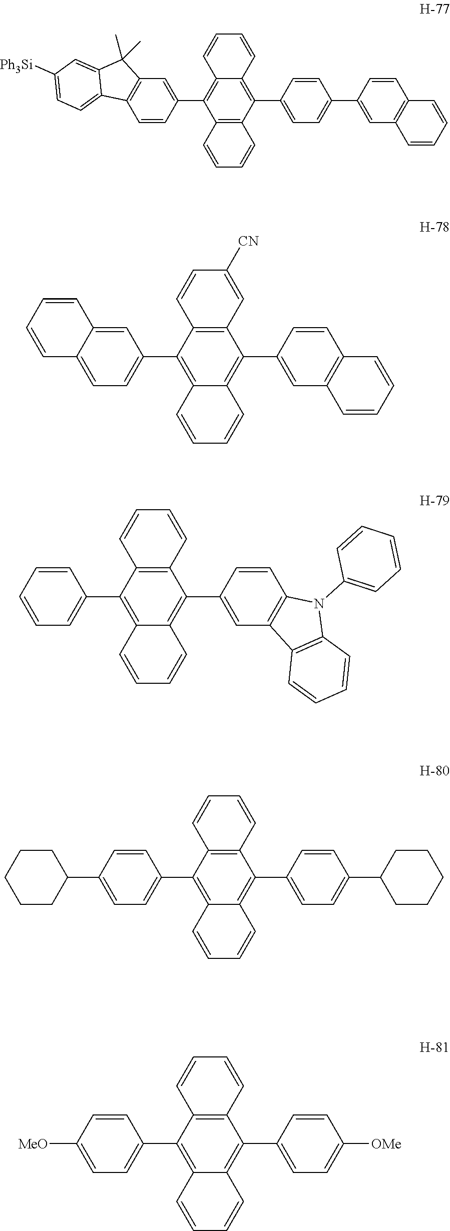

As the dopant comprised in the organic electroluminescent device of the present invention, one or more fluorescent dopants are preferable. A fused polycyclic amine derivative of formula 12 below can be used.

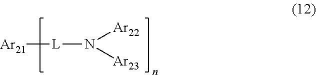

wherein Ar₂₁ represents a substituted or unsubstituted (C6-C50)aryl or a styryl;

L represents a single bond, a substituted or unsubstituted (C6-C30)arylene, or a substituted or unsubstituted 3- to 30-membered heteroarylene;

Ar₂₂ and Ar₂₃ each independently represent hydrogen, deuterium, a halogen, a substituted or unsubstituted (C1-C30)alkyl, a substituted or unsubstituted (C6-C30)aryl, or a substituted or unsubstituted 3- to 30-membered heteroaryl; or are linked to each other to form a mono- or polycyclic, (C3-C30) alicyclic or aromatic ring, whose carbon atom(s) may be replaced with at least one hetero atom selected from nitrogen, oxygen, and sulfur;

n represents 1 or 2, where n is 2, each of

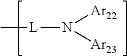

are the same or different.

The preferable aryl groups of Ar₂₁ are a substituted or unsubstituted phenyl, a substituted or unsubstituted fluorenyl, a substituted or unsubstituted anthryl, a substituted or unsubstituted pyrenyl, a substituted or unsubstituted chrysenyl, and a substituted or unsubstituted benzofluorenyl, etc.

Specifically, the fluorescent dopant materials include the following:

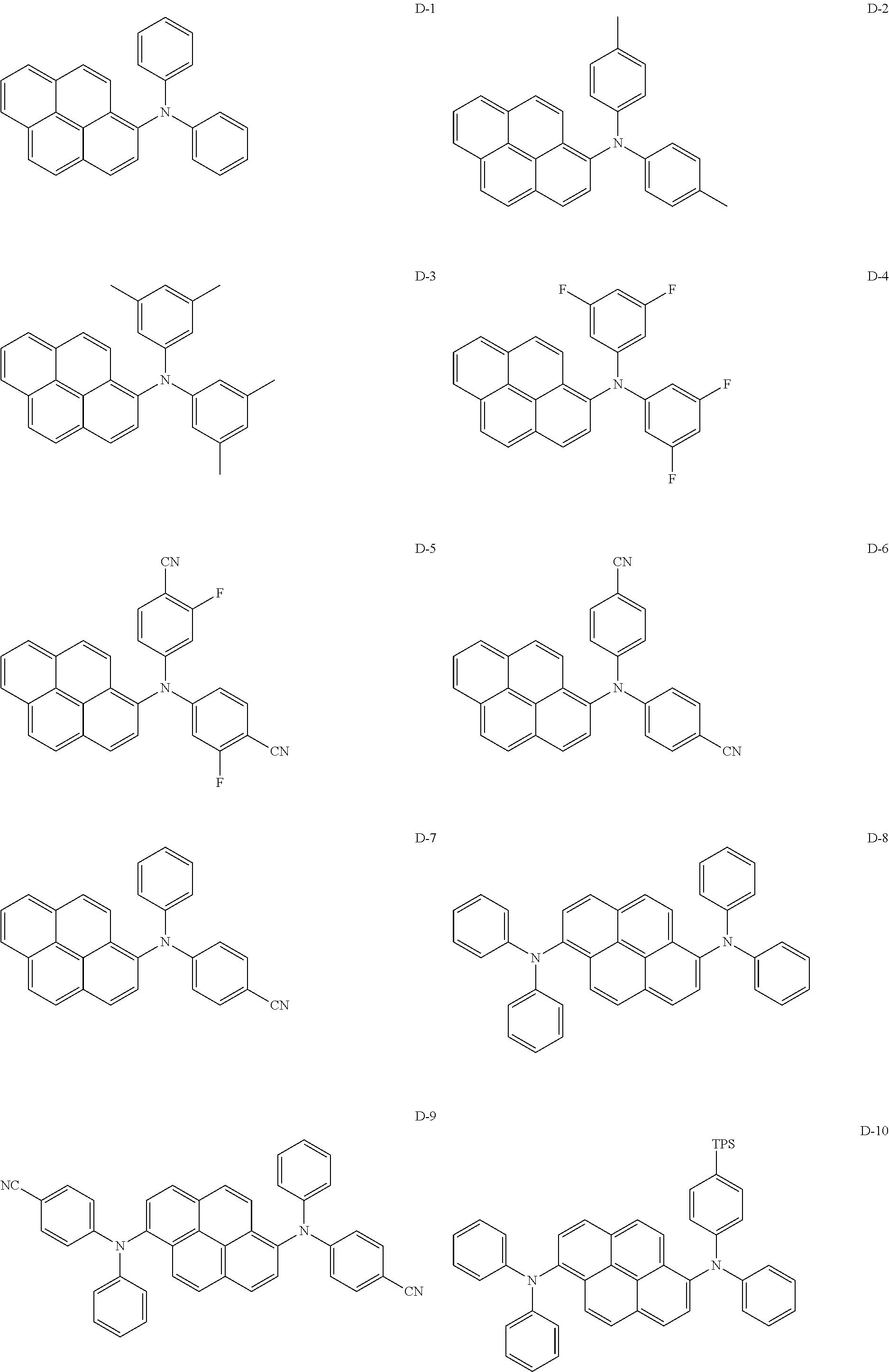

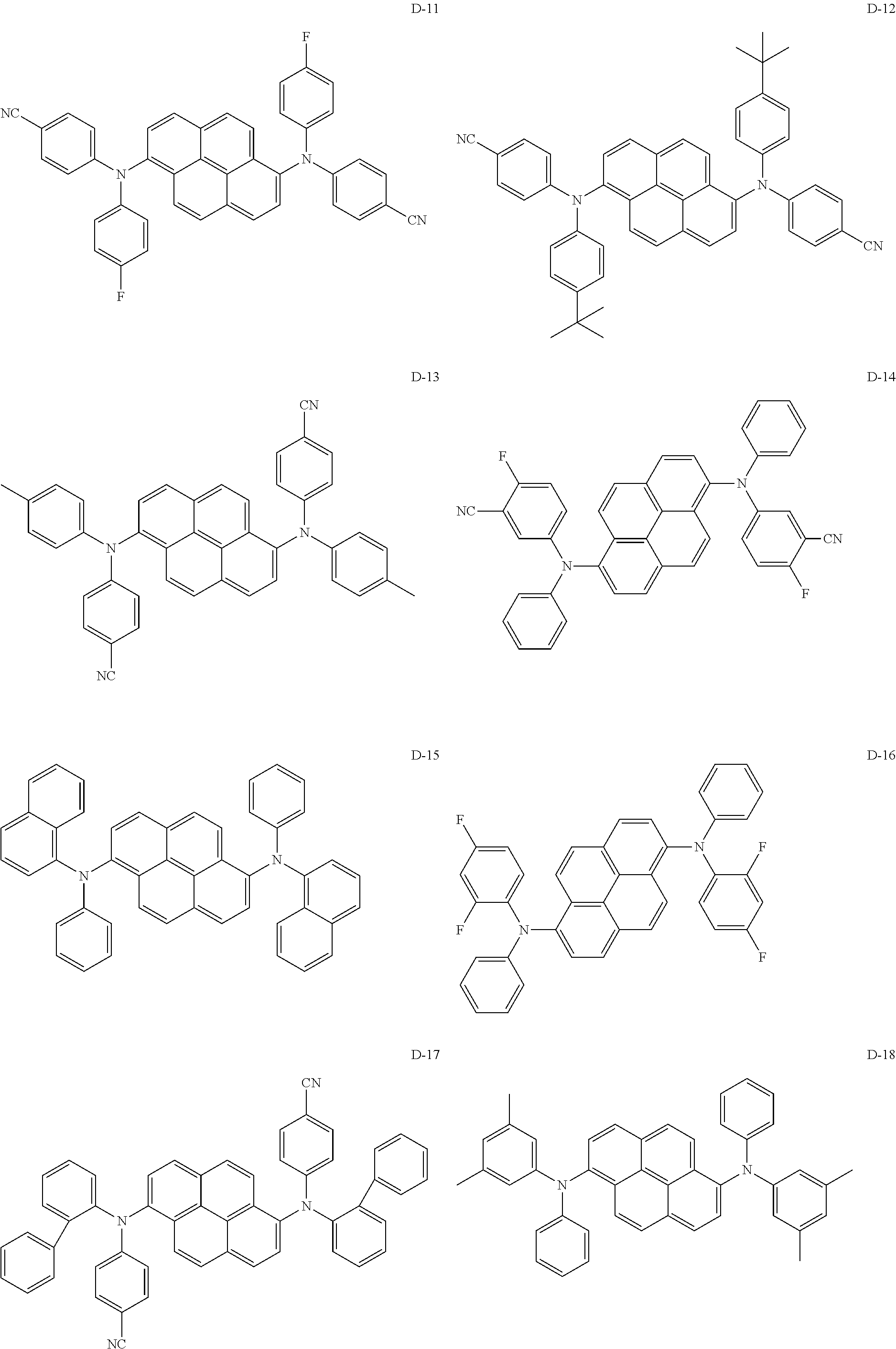

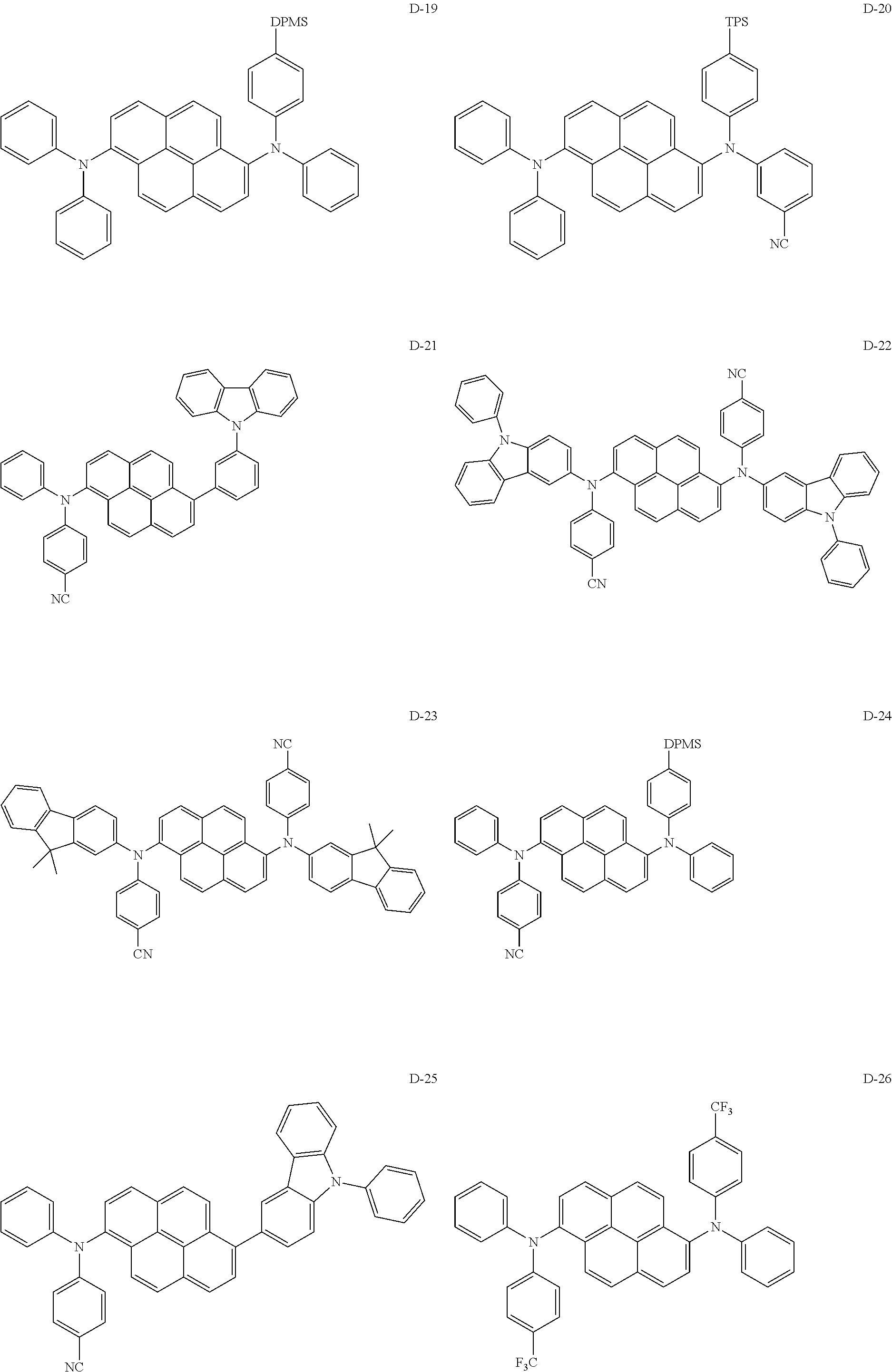

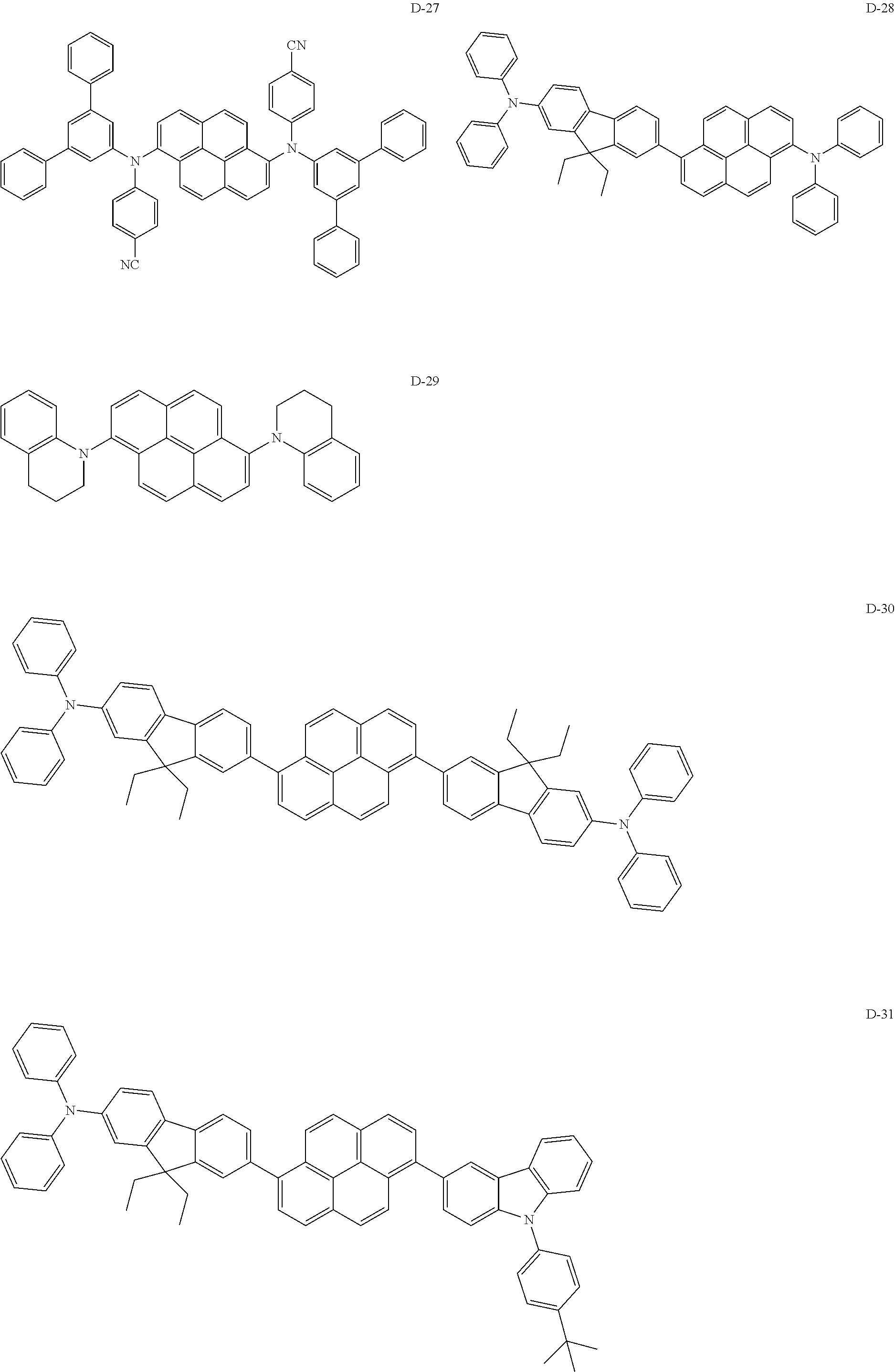

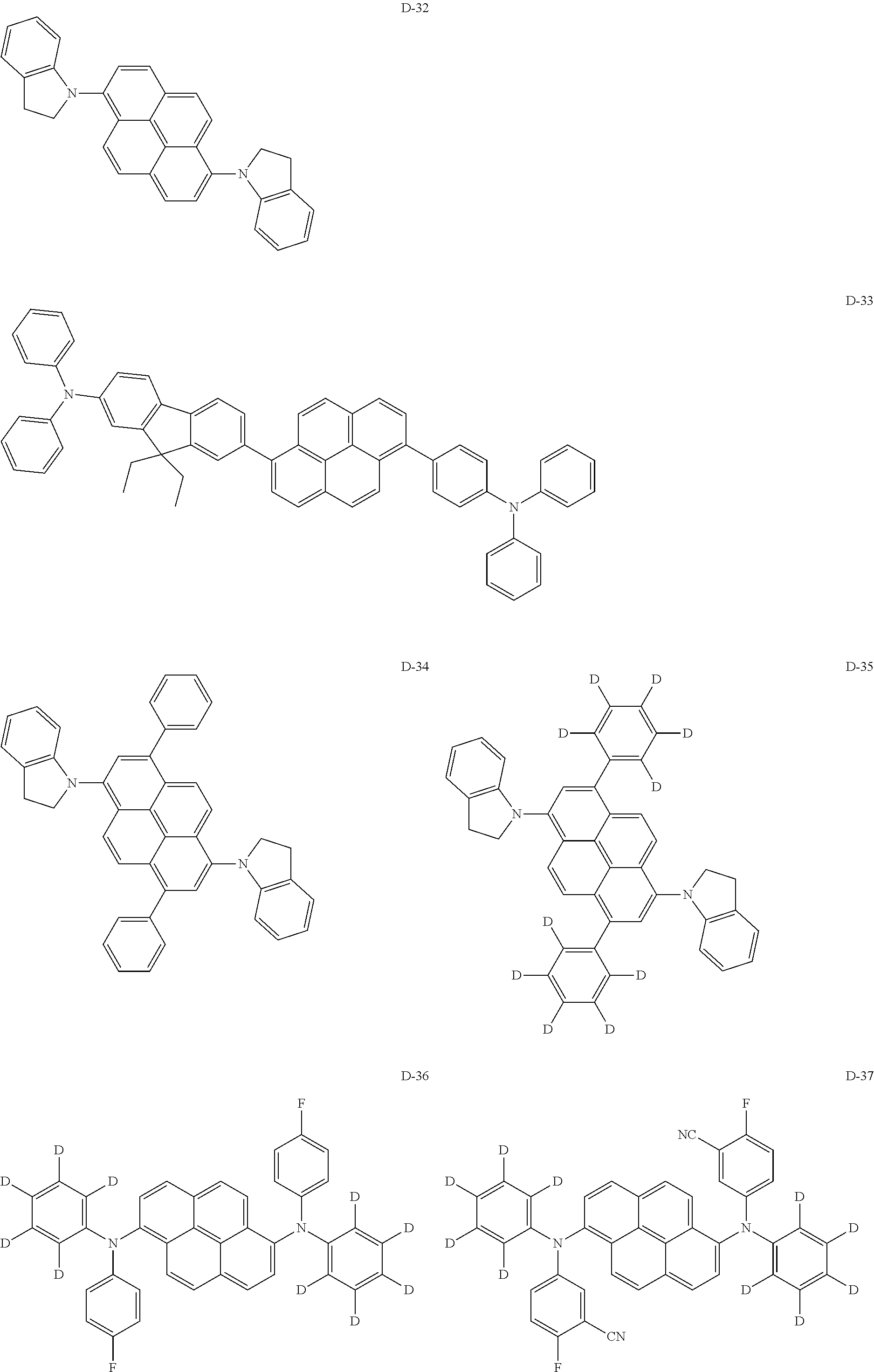

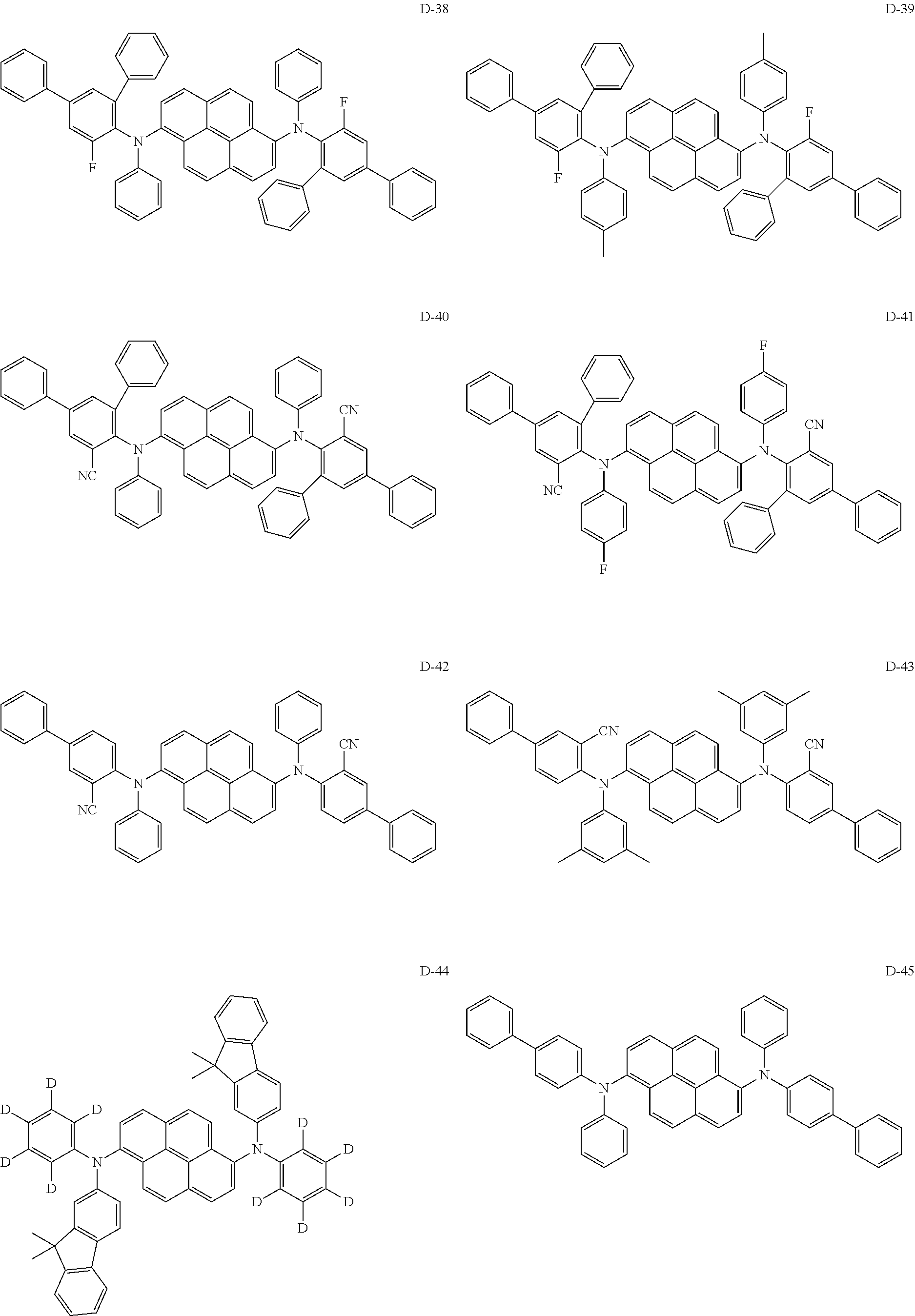

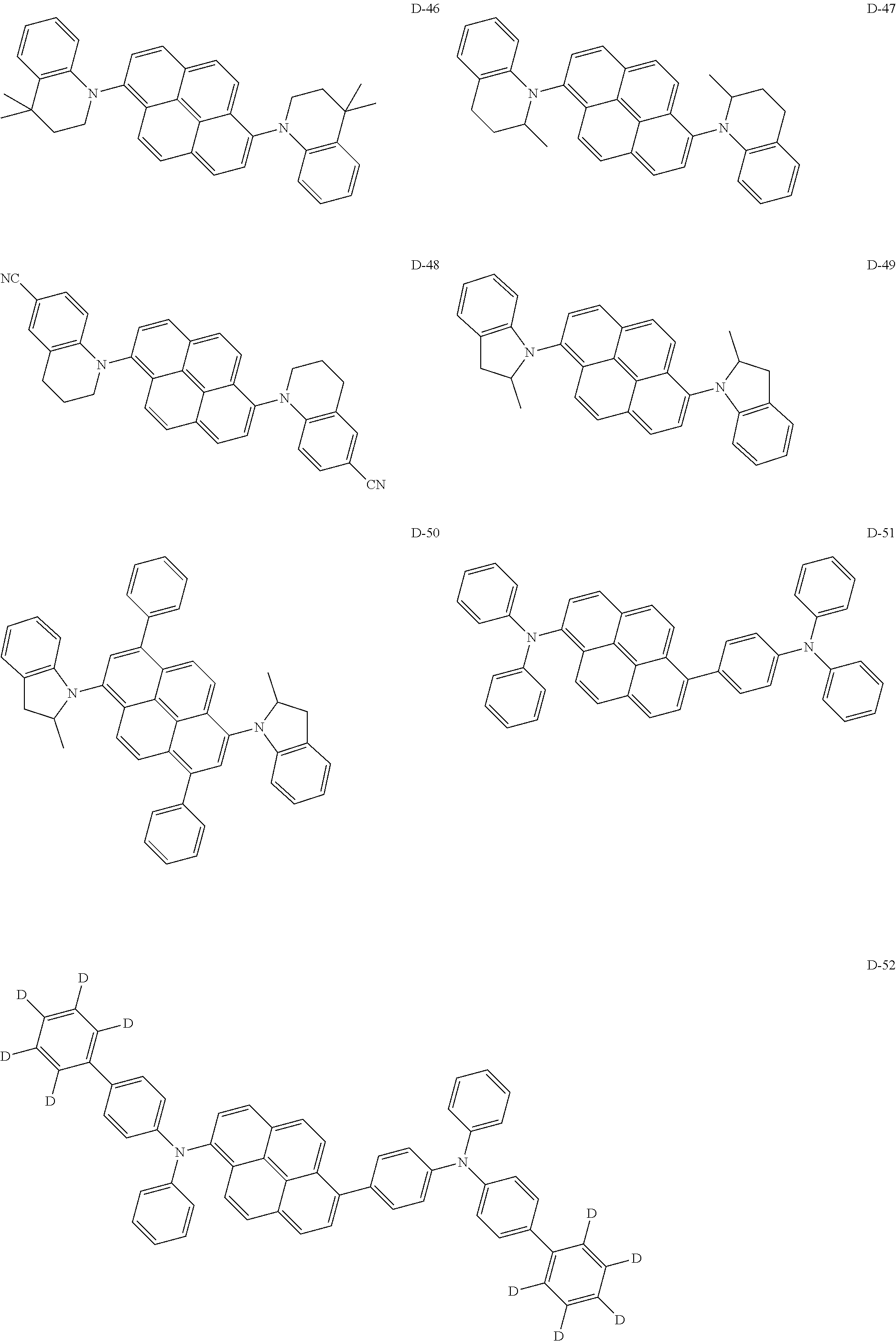

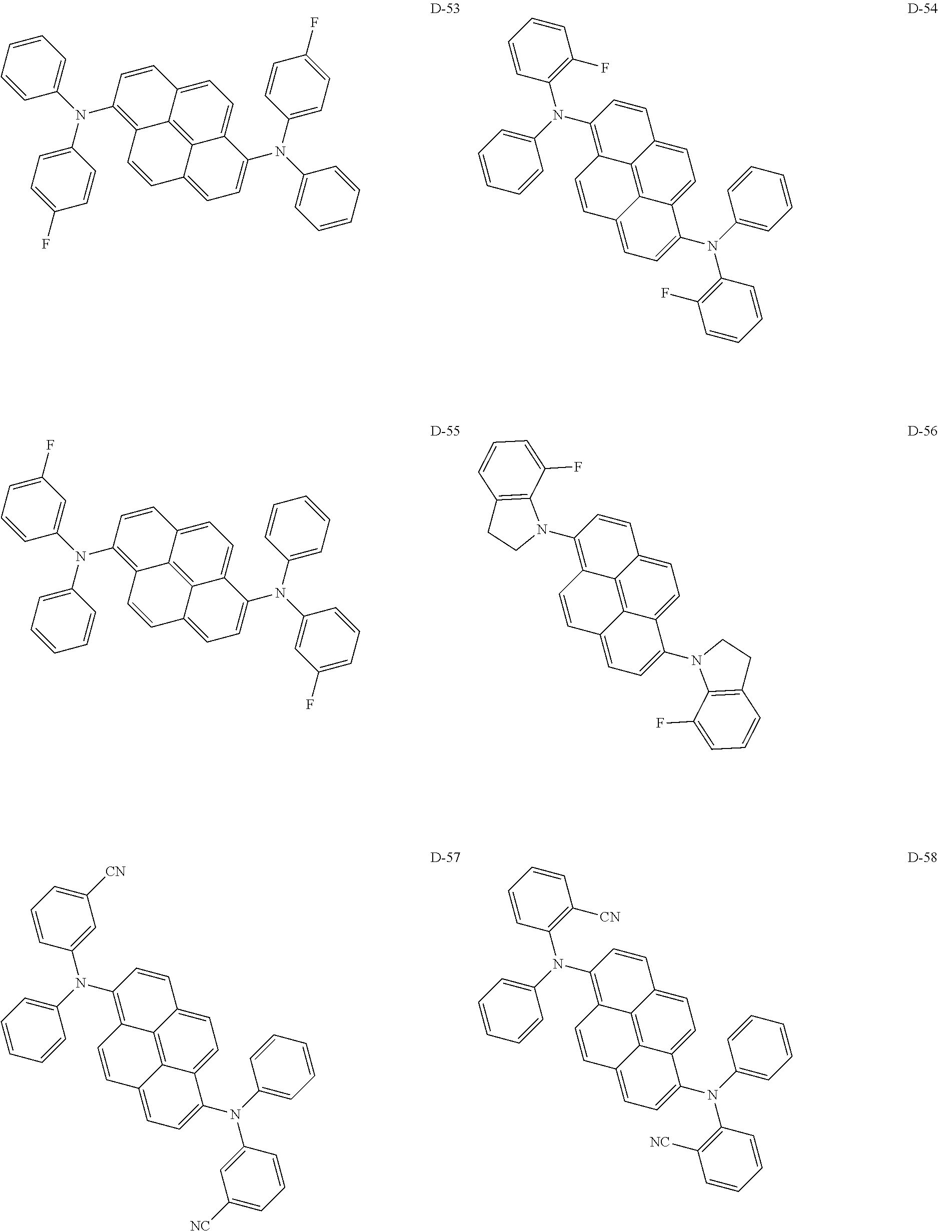 [SMILES: Fc1ccc(N(c2ccccc2)c2ccc3ccc4c(N(c5ccccc5)c5ccc(F)cc5)ccc5ccc2c3c54)cc1 ; Fc1ccccc1N(c1ccccc1)c1ccc2ccc3c(N(c4ccccc4)c4ccccc4F)ccc4ccc1c2c43 ; Fc1cccc(N(c2ccccc2)c2ccc3ccc4c(N(c5ccccc5)c5cccc(F)c5)ccc5ccc2c3c54)c1 ; Fc1cccc2c1N(c1ccc3ccc4c(N5CCc6cccc(F)c65)ccc5ccc1c3c54)CC2 ; N#Cc1cccc(N(c2ccccc2)c2ccc3ccc4c(N(c5ccccc5)c5cccc(C#N)c5)ccc5ccc2c3c54)c1 ; N#Cc1ccccc1N(c1ccccc1)c1ccc2ccc3c(N(c4ccccc4)c4ccccc4C#N)ccc4ccc1c2c43]

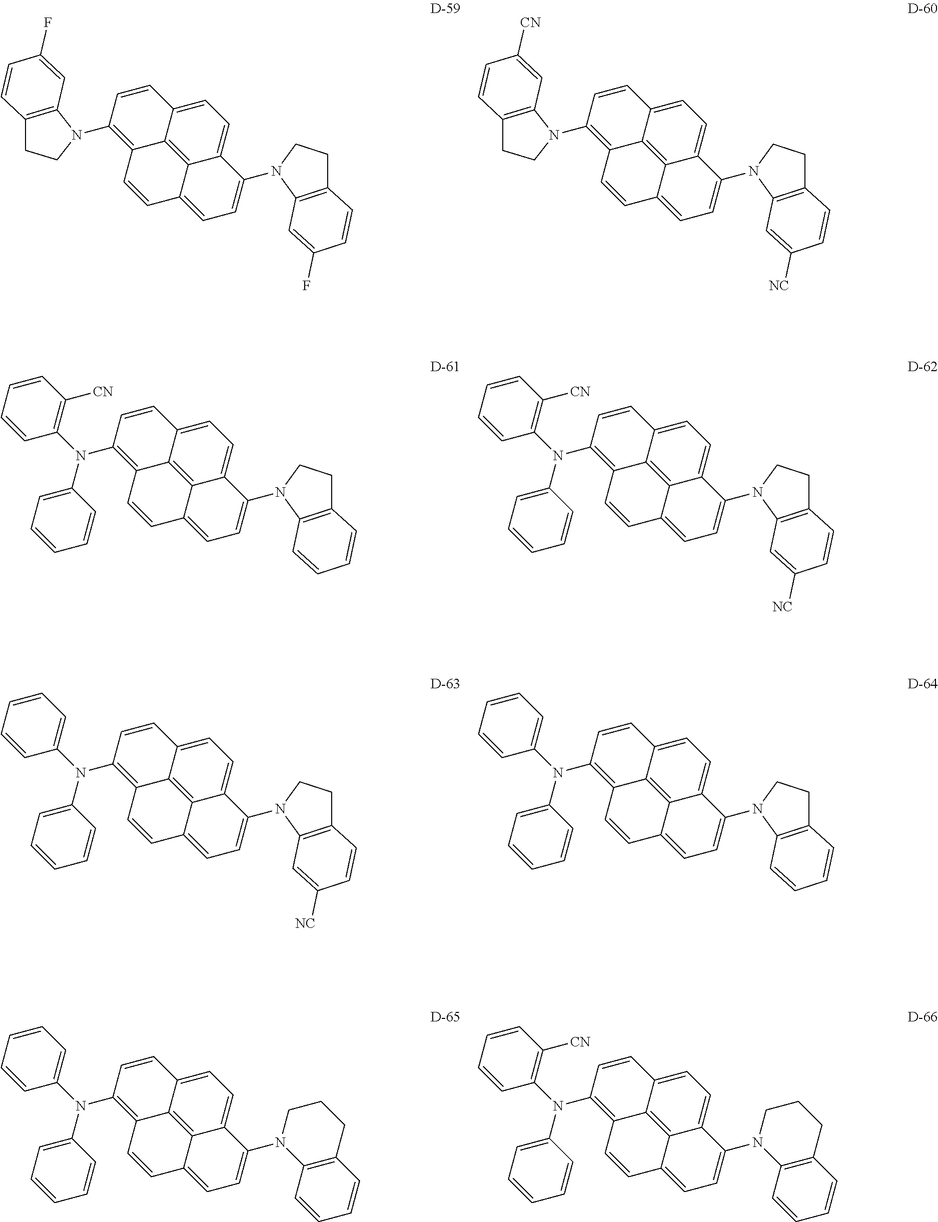

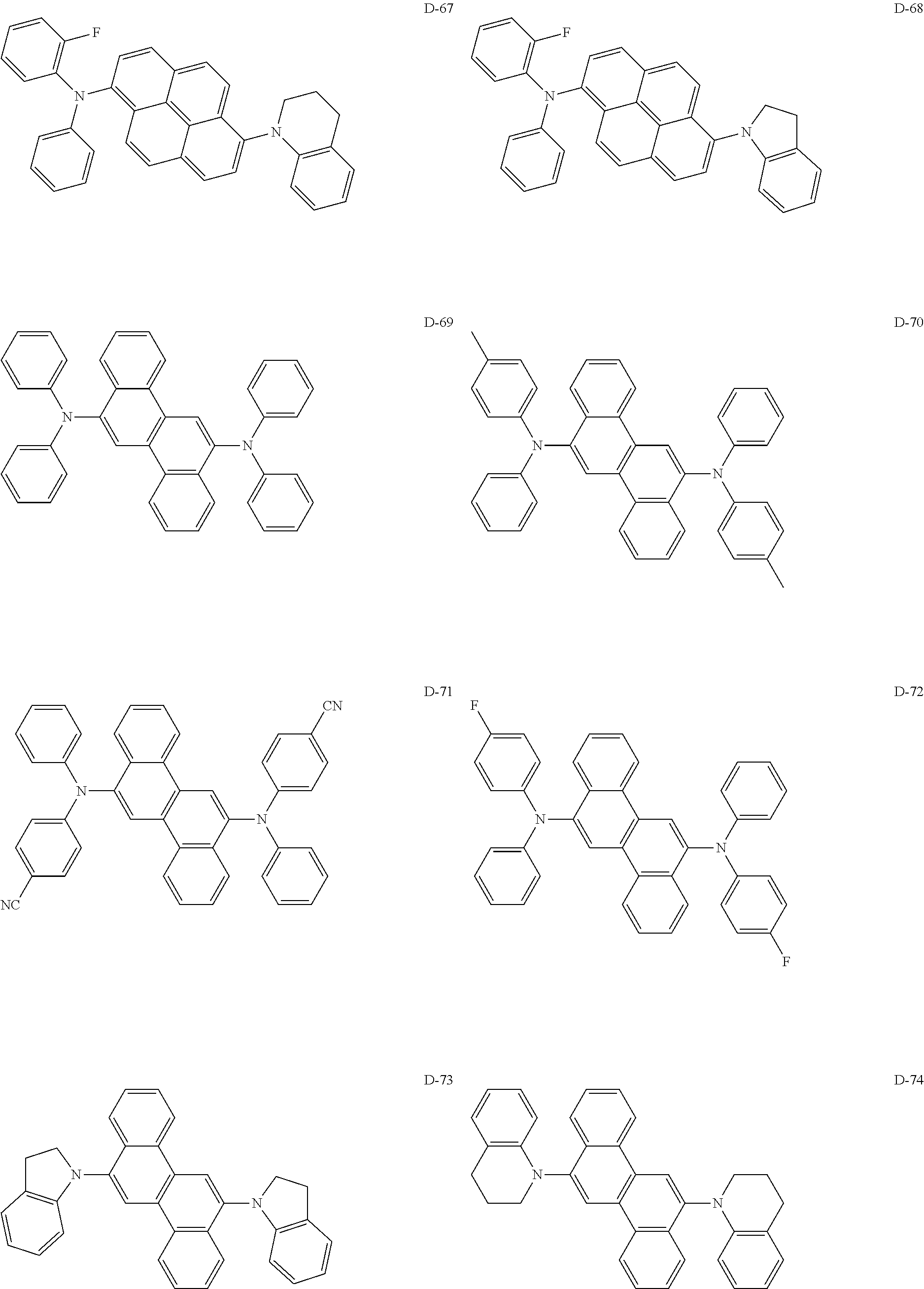

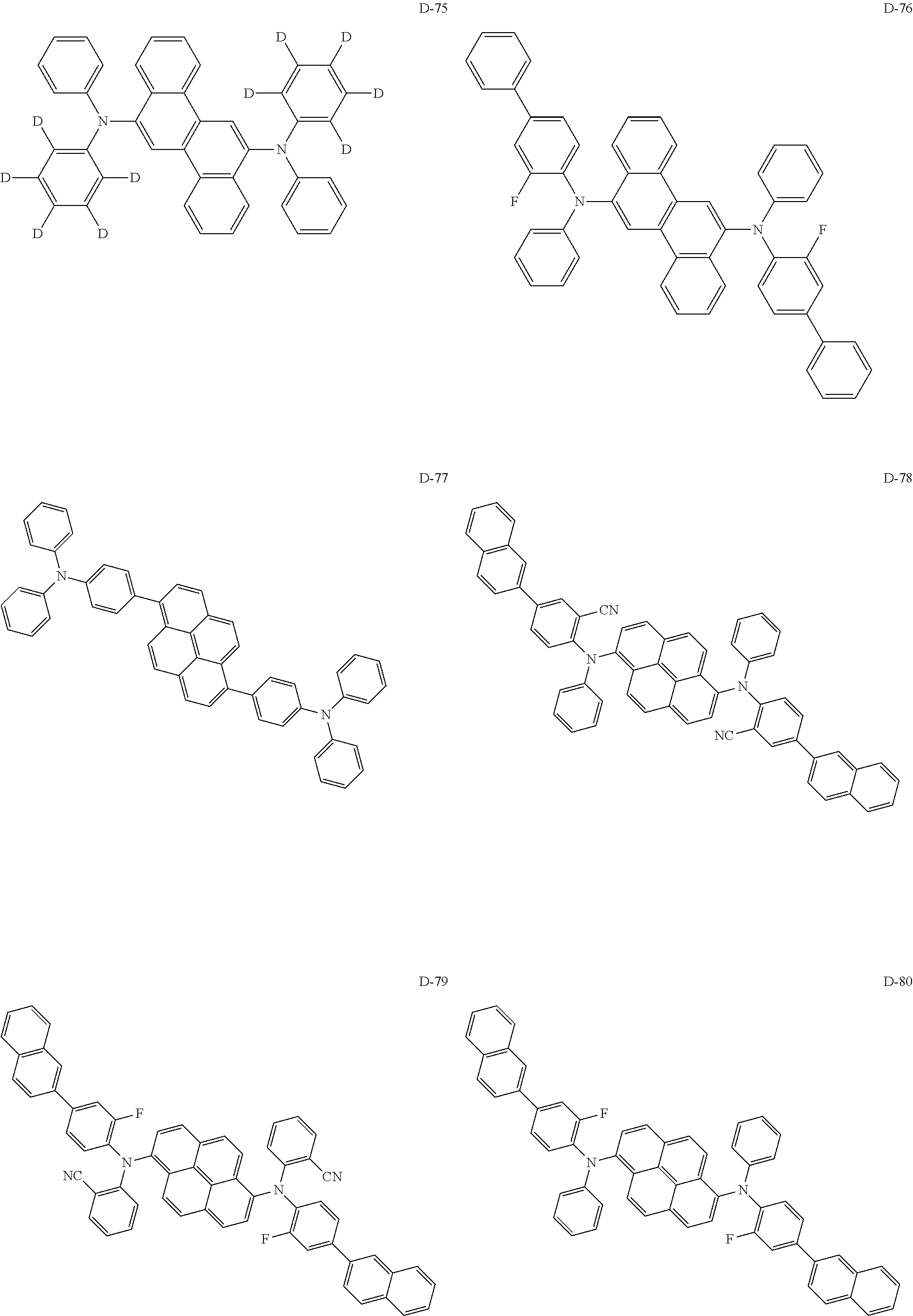

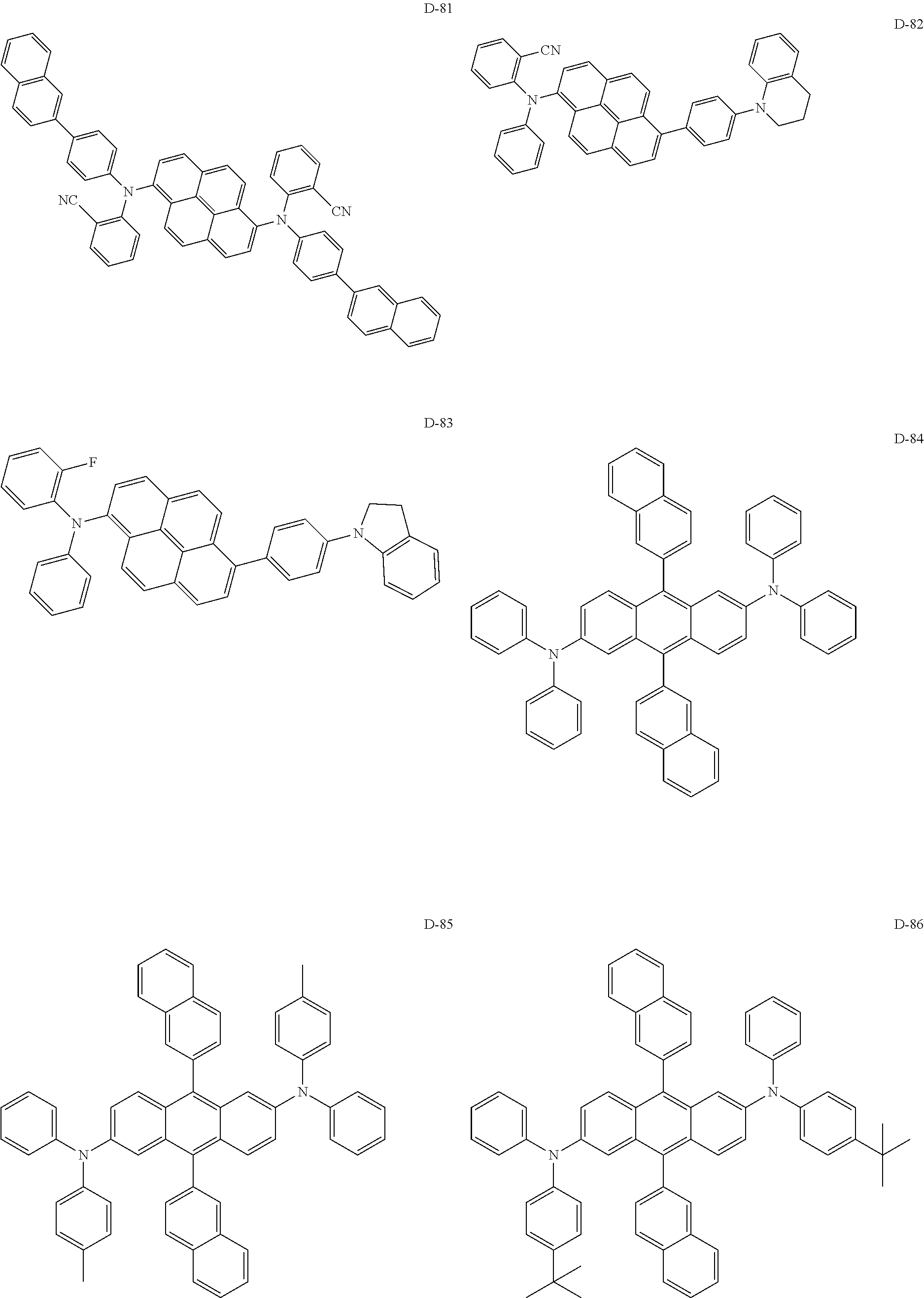

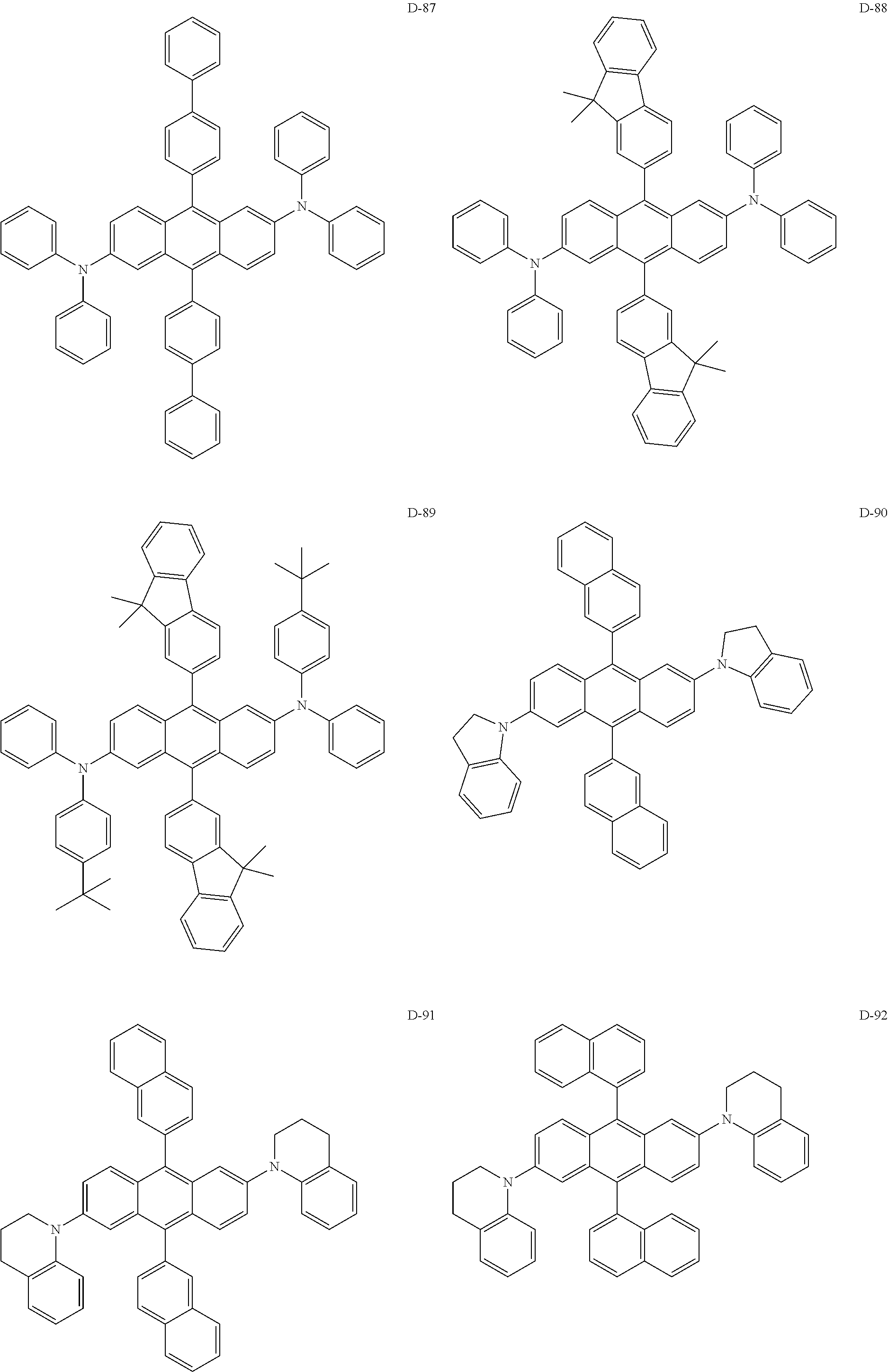

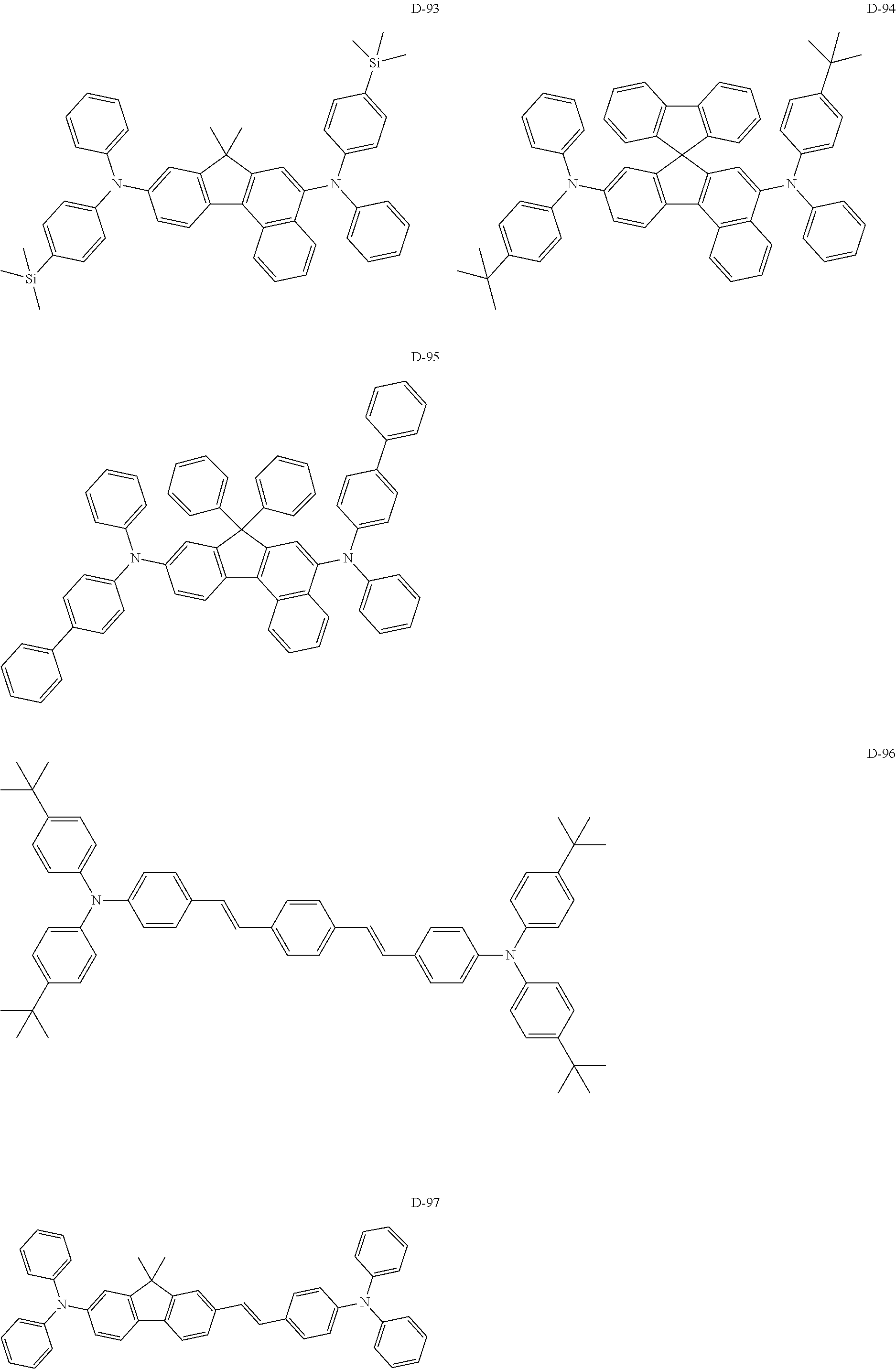

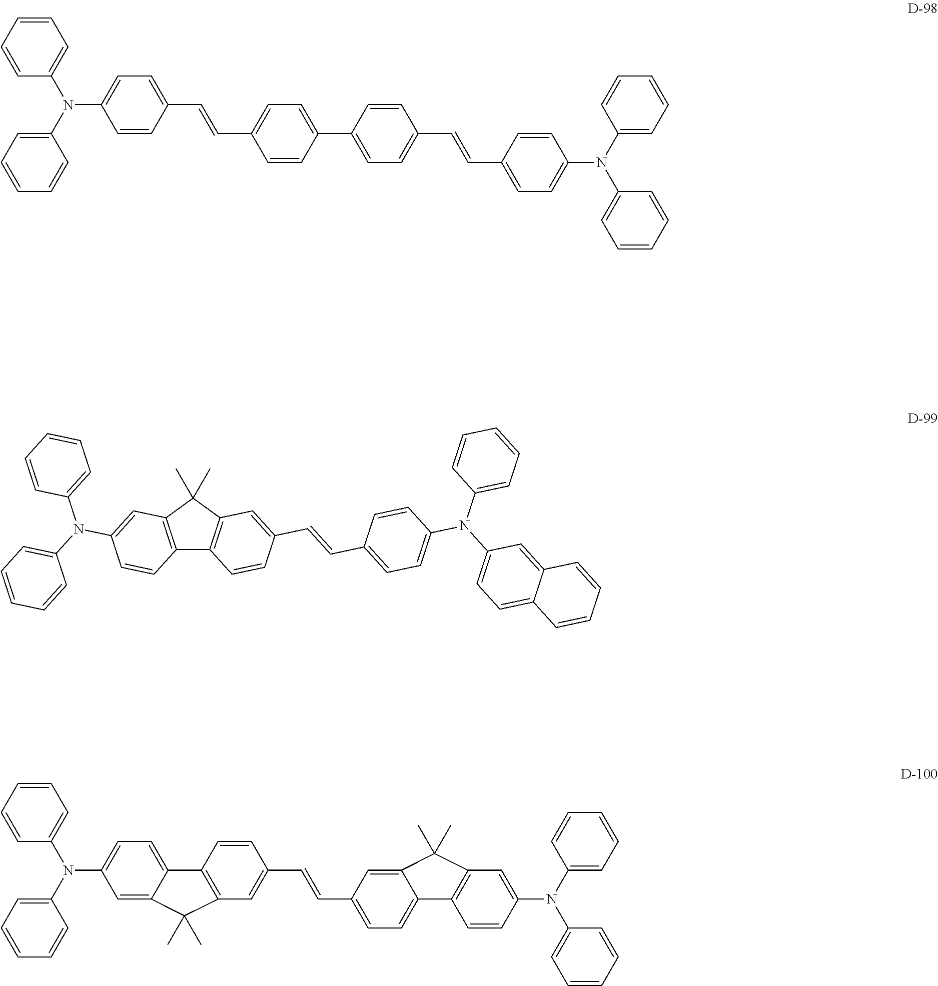 [SMILES: C(=C\c1ccc(N(c2ccccc2)c2ccccc2)cc1)/c1ccc(-c2ccc(/C=C/c3ccc(N(c4ccccc4)c4ccccc4)cc3)cc2)cc1 ; CC1(C)c2cc(/C=C/c3ccc(N(c4ccccc4)c4ccc5ccccc5c4)cc3)ccc2-c2ccc(N(c3ccccc3)c3ccccc3)cc21 ; CC1(C)c2cc(/C=C/c3ccc4c(c3)C(C)(C)c3cc(N(c5ccccc5)c5ccccc5)ccc3-4)ccc2-c2ccc(N(c3ccccc3)c3ccccc3)cc21]

In another embodiment of the present invention, a composition for preparing an organic electroluminescent device is provided. The composition comprises the compound of formula 1 according to the present invention as a hole transport material.

In addition, the organic electroluminescent device according to the present invention comprises a first electrode; a second electrode; and at least one organic layer between the first and second electrodes. The organic layer comprises a hole transport layer, and the hole transport layer may comprise the composition for preparing the organic electroluminescent device according to the present invention.

The organic electroluminescent device according to the present invention may further comprise, in addition to the compound of formula 1, at least one compound selected from the group consisting of arylamine-based compounds and styrylarylamine-based compounds.

In the organic electroluminescent device according to the present invention, the organic layer may further comprise at least one metal selected from the group consisting of metals of Group 1, metals of Group 2, transition metals of the 4^{th} period, transition metals of the 5^{th} period, lanthanides and organic metals of d-transition elements of the Periodic Table, or at least one complex compound comprising said metal. The organic layer may further comprise a light-emitting layer and a charge generating layer.

In addition, the organic electroluminescent device according to the present invention may emit white light by further comprising at least one light-emitting layer which comprises a blue electroluminescent compound, a red electroluminescent compound or a green electroluminescent compound known in the field, besides the compound of formula 1. Also, if needed, a yellow or orange light-emitting layer can be comprised in the device.

According to the present invention, at least one layer (hereinafter, “a surface layer”) is preferably placed on an inner surface(s) of one or both electrode(s); selected from a chalcogenide layer, a metal halide layer and a metal oxide layer. Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, said chalcogenide includes SiO_{X}(1≦X≦2), AlO_{X}(1≦X≦1.5), SiON, SiAlON, etc.; said metal halide includes LiF, MgF₂, CaF₂, a rare earth metal fluoride, etc.; and said metal oxide includes Cs₂O, Li₂O, MgO, Sro, Bao, CaO, etc.

In the organic electroluminescent device according to the present invention, a mixed region of an electron transport compound and a reductive dopant, or a mixed region of a hole transport compound and an oxidative dopant is preferably placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge generating layer to prepare an electroluminescent device having two or more electroluminescent layers and emitting white light.

In order to form each layer of the organic electroluminescent device according to the present invention, dry film-forming methods such as vacuum evaporation, sputtering, plasma and ion plating methods, or wet film-forming methods such as spin coating, dip coating, and flow coating methods can be used.

When using a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

Hereinafter, the compound of formula 1, the preparation method of the compound, and the luminescent properties of the device will be explained in detail with reference to the following examples.

### EXAMPLE 1: PREPARATION OF COMPOUND A-1

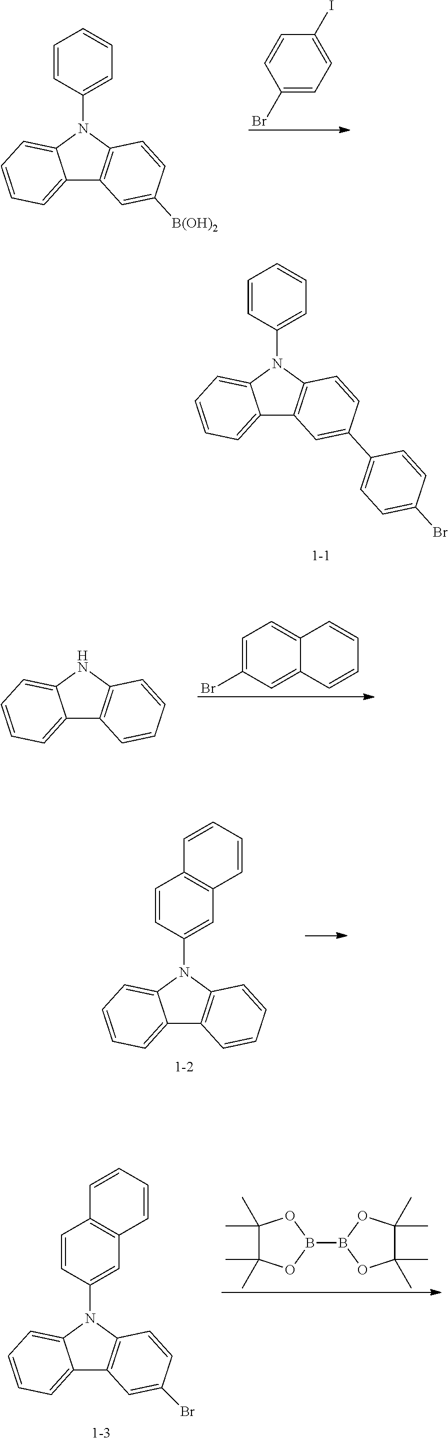

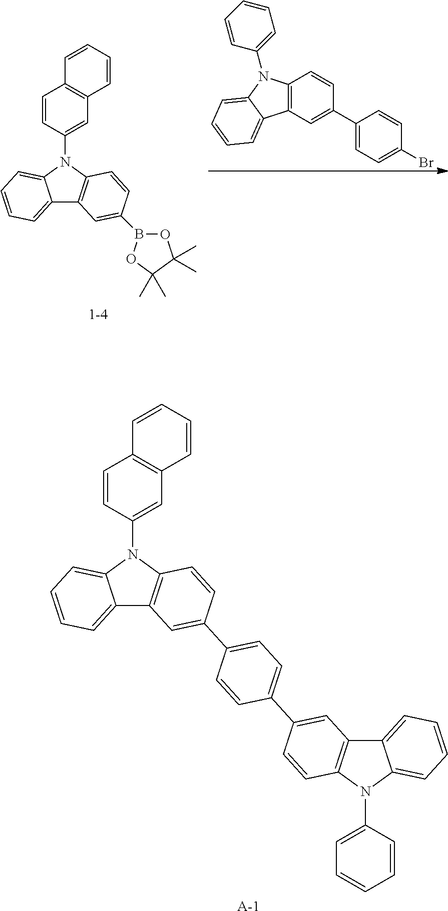 [SMILES: CC1(C)OB(c2ccc3c(c2)c2ccccc2n3-c2ccc3ccccc3c2)OC1(C)C ; Brc1ccc(-c2ccc3c(c2)c2ccccc2n3-c2ccccc2)cc1 ; c1ccc(-n2c3ccccc3c3cc(-c4ccc(-c5ccc6c(c5)c5ccccc5n6-c5ccc6ccccc6c5)cc4)ccc32)cc1 ; "A-1"]

Preparation of Compound 1-1

After introducing (9-phenyl-9H-carbazol-3-yl)boronic acid (30 g, 104.49 mmol), 1-bromo-4-iodobenzene (30 g, 104.49 mmol), tetrakis(triphenylphosphine)palladium (3.6 g, 3.13 mmol), sodium carbonate (28 g, 261.23 mmol), toluene 520 mL, ethanol 130 mL, and distilled water 130 mL in a reaction vessel, the mixture was stirred at 120° C. for 4 hours. After the reaction, the mixture was washed with distilled water, and an organic layer was extracted with ethyl acetate. The extracted organic layer was dried with magnesium sulfate, and the solvent was removed using a rotary evaporator. The remaining product was then purified with column chromatography to obtain compound 1-1 (27 g, yield: 65%).

Preparation of Compound 1-2

After introducing carbazole (20 g, 120 mmol), 2-bromonaphthalene (30 g, 143 mmol), copper(I) iodide (11.7 g, 59.81 mmol), ethylene diamine (8 mL, 120 mmol), potassium phosphate (64 g, 299 mmol), and toluene 600 mL in a reaction vessel, the mixture was stirred at 120° C. for 8 hours. After the reaction, the mixture was washed with distilled water, and an organic layer was extracted with ethyl acetate. The extracted organic layer was dried with magnesium sulfate, and the solvent was removed using a rotary evaporator. The remaining product was then purified with column chromatography to obtain compound 1-2 (13 g, yield: 37%).

Preparation of Compound 1-3

Compound 1-2 (13 g, 44 mmol) was dissolved in dimethylformamide in a reaction vessel. After dissolving N-bromosuccinamide in dimethylformamide, it was introduced to the mixture. After stirring the mixture for 4 hours, the mixture was washed with distilled water, and an organic layer was extracted with ethyl acetate. The extracted organic layer was dried with magnesium sulfate, and the solvent was removed using a rotary evaporator. The remaining product was then purified with column chromatography to obtain compound 1-3 (14 g, yield: 83%).

Preparation of Compound 1-4

After introducing compound 1-3 (14 g, 36 mmol), bis(pinacolato)diborane (11 g, 44 mmol), dichloro-di(triphenylphosphine)palladium (1.3 g, 2 mmol), potassium acetate (9 g, 91 mmol), and 1,4-dioxane 180 mL in a reaction vessel, the mixture was stirred at 140° C. for 2 hours. After the reaction, the mixture was washed with distilled water, and an organic layer was extracted with ethyl acetate. The extracted organic layer was dried with magnesium sulfate, and the solvent was removed using a rotary evaporator. The remaining product was then purified with column chromatography to obtain compound 1-4 (8 g, yield: 52%).

Preparation of Compound A-1

After introducing compound 1-1 (7 g, 17 mmol), compound 1-4 (8 g, 19 mmol), tetrakis(triphenylphosphine)palladium (0.6 g, 0.5 mmol), sodium carbonate (4.5 g, 43 mmol), toluene 100 mL, ethanol 25 mL, and distilled water 25 mL in a reaction vessel, the mixture was stirred at 120° C. for 4 hours. After the reaction, the mixture was washed with distilled water, and an organic layer was extracted with ethyl acetate. The extracted organic layer was dried with magnesium sulfate, and the solvent was removed using a rotary evaporator. The remaining product was then purified with column chromatography to obtain compound A-1 (4 g, yield: 87%).

| | MW | UV | PL | M.P |
|---|---|---|---|---|
| A-1 | 610.74 | 354 nm | 397 nm | 198° C. |

### EXAMPLE 2: PREPARATION OF COMPOUND A-4

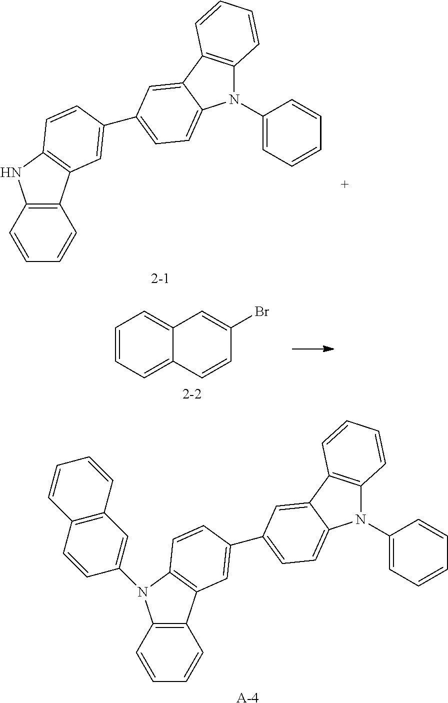 [SMILES: c1ccc(-n2c3ccccc3c3cc(-c4ccc5[nH]c6ccccc6c5c4)ccc32)cc1 ; Brc1ccc2ccccc2c1 ; c1ccc(-n2c3ccccc3c3cc(-c4ccc5c(c4)c4ccccc4n5-c4ccc5ccccc5c4)ccc32)cc1 ; "A-4"]

Preparation of Compound A-4

After dissolving compound 2-1 (9-phenyl-9H, 9′H-3,3′-bicarbazole) (15 g, 36.70 mmol), compound 2-2 (2-bromonaphthalene) (7.6 g, 36.70 mmol), Pd₂(dba)₃ (1.0 g, 1.10 mmol), P(t-Bu)₃ (3.7 mL, 2.20 mmol), and NaOtBu (5.3 g, 55.10 mmol) in toluene 200 mL in a flask, the mixture was stirred under reflux at 120° C. for 4 hours. After the reaction, the mixture was separated with column chromatography, and methanol was added thereto. The produced solid was filtered under reduced pressure. The produced solid was recrystallized with toluene to obtain compound A-4 (13.5 g, yield: 69%).

| | MW | UV | PL | M.P |
|---|---|---|---|---|
| A-4 | 534.65 | 368 nm | 407 nm | 186.5° C. |

### EXAMPLE 3: PREPARATION OF COMPOUND A-7

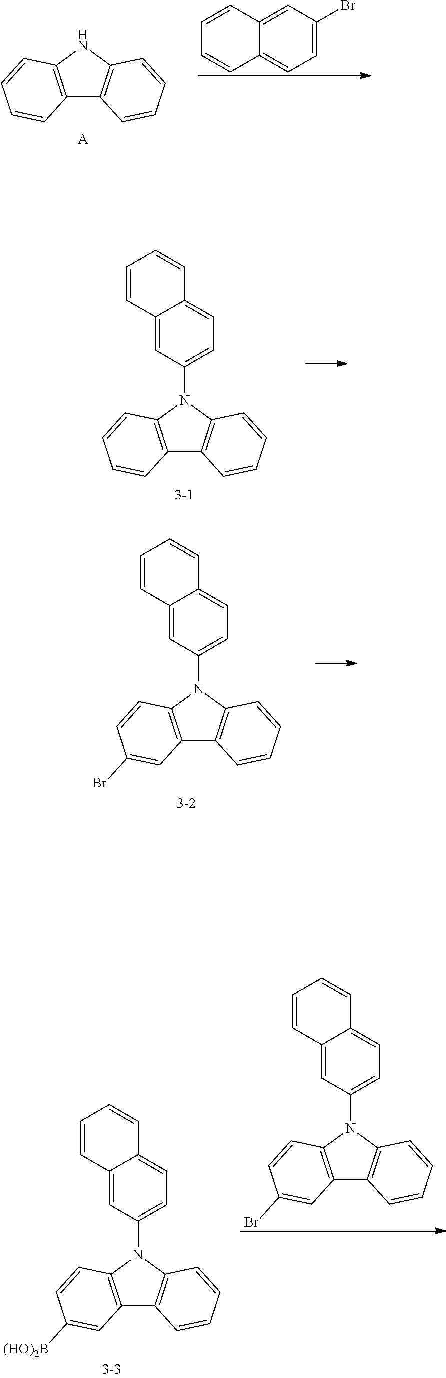

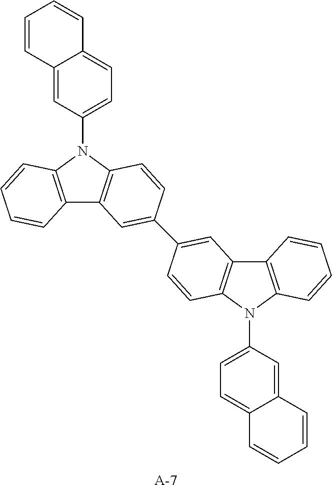 [SMILES: c1ccc2cc(-n3c4ccccc4c4cc(-c5ccc6c(c5)c5ccccc5n6-c5ccc6ccccc6c5)ccc43)ccc2c1 ; "A-7"]

Preparation of Compound 3-1

After dissolving 9H-carbazole (20 g, 119.60 mmol), 2-bromonaphthalene (37 g, 179.46 mmol), CuI (11 g, 59.8 mmol), ethylene diamine (8 mL, 119.6 mmol), and K₃PO₄ (50 g, 239.2 mmol) in toluene 598 mL in a flask, the mixture was stirred under reflux at 120° C. for 5 hours. After the reaction, an organic layer was extracted with ethyl acetate, the residual moisture was removed using magnesium sulfate, and dried. The remaining product was then separated with column chromatography to obtain compound 3-1 (24.4 g, yield: 70%).

Preparation of Compound 3-2

After dissolving compound 3-1 (9-(naphthalene-2-yl)-carbazole) (24 g, 93.2 mmol) and N-bromosuccinimide (14 g, 79 mmol) in tetrahydrofuran (THF) 832 mL, the mixture was stirred at room temperature for 20 hours. After the reaction, an organic layer was extracted with ethyl acetate, the residual moisture was removed using magnesium sulfate, and dried. The remaining product was then separated with column chromatography to obtain compound 3-2 (26.4 g, yield: 84%).

Preparation of Compound 3-3

After dissolving compound 3-2 (3-bromo-9-(naphthalen-2-yl)-carbazole (16 g, 43 mmol) in THF 400 mL, the mixture was cooled to −78° C. 2.5 M n-butyl lithium (21 mL, 51.6 mmol) was then added to the mixture, and stirred for 1 hour. Triisopropyl borate (15 mL, 66 mmol) was then added to the mixture, and reacted for 8 hours. After the reaction, the produced white solid was filtered to obtain compound 3-3 (8.7 g, yield: 50%).

Preparation of Compound A-7

After dissolving compound 3-2 (3-bromo-9-(naphthalen-2-yl)-carbazole (8 g, 21.5 mmol), compound 3-3 ((9-(naphthalen-2-yl)-9H-carbazol-3-yl)boronic acid) (8.7 g, 25.8 mmol), and tetrakis(triphenylphosphine)palladium(O) (Pd(PPh₃)₄) (993 mg, 0.86 mmol) in a mixed solvent of 2M K₂CO₃ 27 mL, toluene 108 mL, and ethanol 27 mL, the mixture was stirred under reflux at 120° C. for 2 hours. After the reaction, an organic layer was extracted with ethyl acetate, the residual moisture was removed using magnesium sulfate, and dried. The remaining product was then separated with column chromatography to obtain compound A-7 (1.5 g, yield: 12%).

| | MW | UV | PL | M.P |
|---|---|---|---|---|
| A-7 | 584.71 | 306 nm | 407 nm | 301° C. |

### EXAMPLE 4: PREPARATION OF COMPOUND A-15

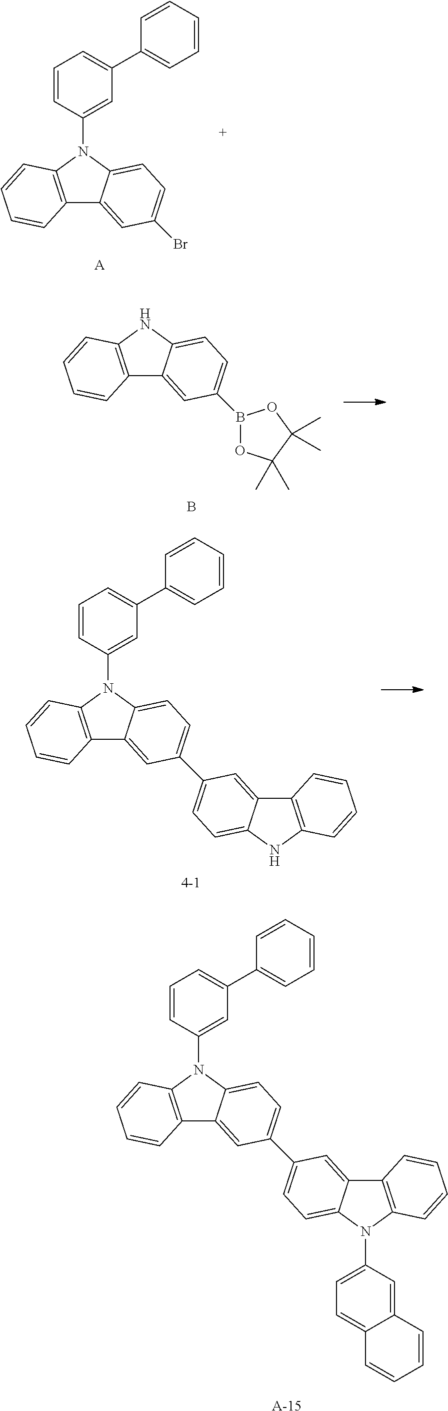

Preparation of Compound 4-1

After dissolving 9-[1,1′-phenyl]-3-yl-3-bromo-9H-carbazole (12 g, 31.8 mmol), 3-(4,4,5,5-tetramethyl-1,3,2-dioxaborolan-2-yl)-9H-carbazole (9.3 g, 31.8 mmol), and tetrakis(triphenylphosphine)palladium(O) (Pd(PPh₃)₄) (1.1 g, 0.95 mmol) in a mixed solvent of 2M K₂CO₃ 40 mL, toluene 160 mL, and ethanol 40 mL, the mixture was stirred under reflux for 4 hours. After the reaction, an organic layer was extracted with ethyl acetate, the residual moisture was removed using magnesium sulfate, and dried. The remaining product was then separated with column chromatography to obtain compound 4-1 (9.5 g, yield: 63%).

Preparation of Compound A-15

After introducing compound 4-1 (7 g, 14.4 mmol), 2-bromonaphthalene (3.3 g, 15.8 mmol), tris(dibenzylideneacetone)dipalladium (0.6 g, 0.72 mmol), tri-tert-butylphosphine (0.7 mL (50%), 1.44 mmol), sodium tert-butoxide (3.4 g, 36.1 mmol), and toluene 80 mL in a flask, the mixture was stirred under reflux for 2.5 hours. After cooling the mixture to room temperature, distilled water was added thereto. The mixture was extracted with methylene chloride, and dried with magnesium sulfate. The remaining product was then filtered under reduced pressure, and separated with column chromatography to obtain compound A-15 (6.7 g, yield: 76%).

| | MW | UV | PL | M.P |
|---|---|---|---|---|
| A-15 | 610.74 | 352 nm | 406 nm | 192° C. |

### DEVICE EXAMPLES 1 TO 4: PRODUCTION OF AN OLED DEVICE ACCORDING TO THE PRESENT INVENTION

An OLED device of the present invention was produced as follows: A transparent electrode indium tin oxide (ITO) thin film (10 Ω/sq) on a glass substrate for an organic light-emitting diode (OLED) device (Geomatec, Japan) was subjected to an ultrasonic washing with acetone and isopropan alcohol, sequentially, and then was stored in isopropan alcohol. The ITO substrate was then mounted on a substrate holder of a vacuum vapor depositing apparatus. Compound HI-1 was introduced into a cell of said vacuum vapor depositing apparatus, and then the pressure in the chamber of said apparatus was controlled to 10⁻⁶ torr. Thereafter, an electric current was applied to the cell to evaporate the above introduced material, thereby forming a first hole injection layer having a thickness of 60 nm on the ITO substrate. Compound HI-2 was then introduced into another cell of said vacuum vapor depositing apparatus, and was evaporated by applying an electric current to the cell, thereby forming a second hole injection layer having a thickness of 5 nm on the first hole injection layer. Compound HT-1 was then introduced into another cell of said vacuum vapor depositing apparatus, and was evaporated by applying an electric current to the cell, thereby forming a first hole transport layer having a thickness of 20 nm on the second hole injection layer. Next, the compound of formula 1 of the present invention was introduced into another cell of said vacuum vapor depositing apparatus, and was evaporated by applying an electric current to the cell, thereby forming a second hole transport layer having a thickness of 5 nm on the first hole transport layer. Thereafter, compound H-15 was introduced into one cell of the vacuum vapor depositing apparatus, as a host, and compound D-38 was introduced into another cell as a dopant. The two materials were evaporated at different rates and were deposited in a doping amount of 2 wt % based on the total amount of the dopant and host to form a light-emitting layer having a thickness of 20 nm on the second hole transport layer. 2-(4-(9,10-di(naphthalen-2-yl)anthracen-2-yl)phenyl)-1-phenyl-1H-benzo[d]imidazole was introduced into one cell and lithium quinolate was introduced into another cell. The two materials were evaporated at the same rate and were deposited in a doping amount of 50 wt % each to form an electron transport layer having a thickness of 35 nm on the light-emitting layer. After depositing lithium quinolate as an electron injection layer having a thickness of 2 nm on the electron transport layer, an Al cathode having a thickness of 80 nm was then deposited by another vacuum vapor deposition apparatus on the electron injection layer. Thus, an OLED device was produced. All the materials used for producing the OLED device were purified by vacuum sublimation at 10⁻⁶ torr prior to use.

The driving voltage at 1,000 nit of luminance, luminous efficiency, CIE color coordinate, and the time period for the luminance to decrease from 100% to 90% at 2,000 nit and constant current of the organic electroluminescent devices are shown in Table 1 below.

### COMPARATIVE EXAMPLES 1 TO 4: PRODUCTION OF AN OLED DEVICE USING A CONVENTIONAL COMPOUND

An OLED device was produced in the same manner as in Device Example 1, except for using conventional compounds for a hole transport material instead of the compound of formula 1 of the present invention in the second hole transport layer.

The evaluation results of the device of Device Examples 1 to 4 and Comparative Examples 1 to 4 are shown in Tables 1 and 2 below.

TABLE 1

| | Second Hole | | | Color | Color | |
|---|---|---|---|---|---|---|
| | Transport | Voltage | Efficiency | Coordinate | Coordinate | Lifespan |
| | Layer | (V) | (cd/A) | (x) | (y) | (T90hr) |
| Comparative | B-1 | 4.1 | 4.6 | 0.139 | 0.092 | 50 |
| Example 1 | | | | | | |
| Comparative | B-2 | 4.1 | 4.2 | 0.14 | 0.093 | 23 |
| Example 2 | | | | | | |
| Comparative | B-4 | 4.2 | 6 | 0.139 | 0.098 | 50 |
| Example 3 | | | | | | |
| Device | A-4 | 4.3 | 6 | 0.14 | 0.094 | 71.6 |
| Example 1 | | | | | | |
| Device | A-7 | 4.4 | 6.1 | 0.14 | 0.094 | 77 |
| Example 2 | | | | | | |
| Device | A-15 | 4.4 | 6.4 | 0.14 | 0.094 | 64.4 |
| Example 3 | | | | | | |

TABLE 2

| | Second Hole | | | Color | Color | |
|---|---|---|---|---|---|---|
| | Transport | Voltage | Efficiency | Coordinate | Coordinate | Lifespan |
| | Layer | (V) | (cd/A) | (x) | (y) | (T90hr) |
| Comparative | B-3 | 4.3 | 6.2 | 0.139 | 0.098 | 35 |
| Example 4 | | | | | | |
| Device | A-1 | 4.2 | 6.6 | 0.139 | 0.101 | 41 |
| Example 4 | | | | | | |

TABLE 3
The compounds used in the Device Examples and the Comparative
Examples

| | |
|---|---|
| Hole Injection Layer/ Hole Transport Layer | 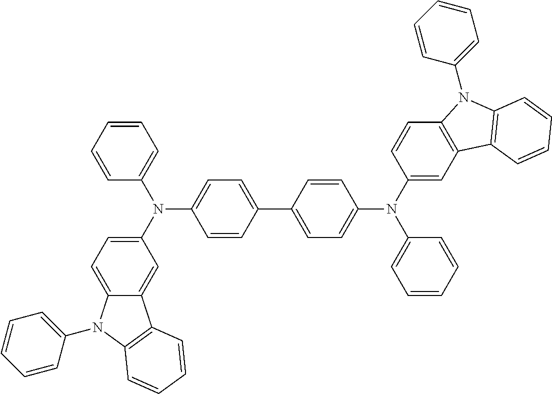 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1] HI-1 |
| | 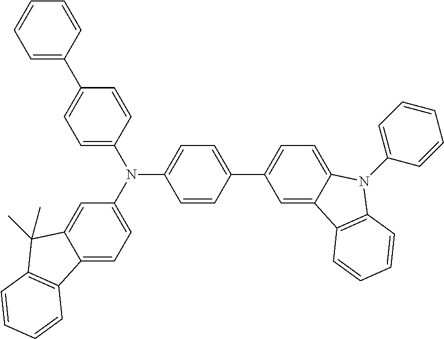 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] HI-2 |
| | 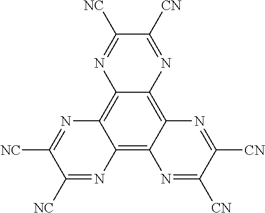 [SMILES: N#Cc1nc2c3nc(C#N)c(C#N)nc3c3nc(C#N)c(C#N)nc3c2nc1C#N] HT-1 |
| Light- Emitting Layer | 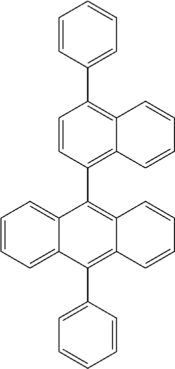 [SMILES: c1ccc(-c2ccc(-c3c4ccccc4c(-c4ccccc4)c4ccccc34)c3ccccc23)cc1] H-15 |
| | 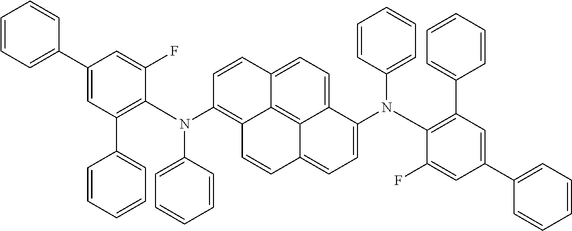 [SMILES: Fc1cc(-c2ccccc2)cc(-c2ccccc2)c1N(c1ccccc1)c1ccc2ccc3c(N(c4ccccc4)c4c(F)cc(-c5ccccc5)cc4-c4ccccc4)ccc4ccc1c2c43] D-38 |
| Comparative Compounds | 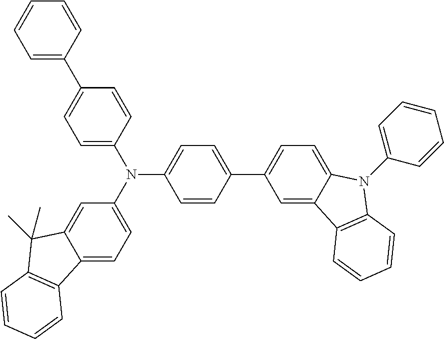 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] B-1 |
| | 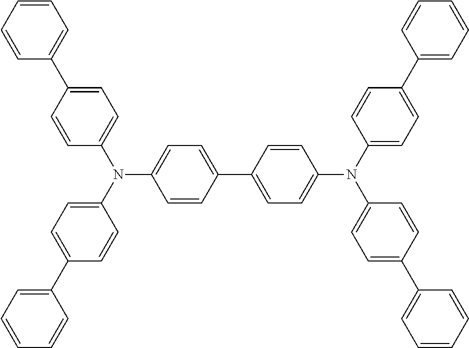 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1] B-2 |
| | 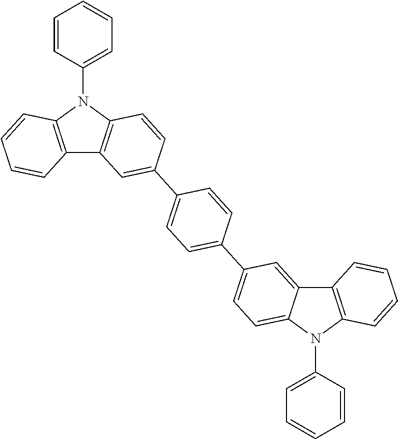 [SMILES: c1ccc(-n2c3ccccc3c3cc(-c4ccc(-c5ccc6c(c5)c5ccccc5n6-c5ccccc5)cc4)ccc32)cc1] B-3 |
| | 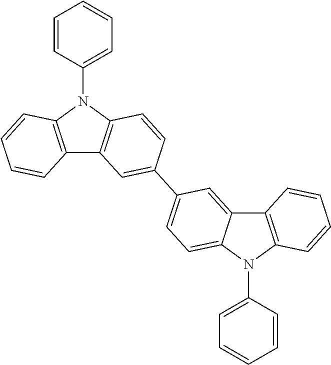 [SMILES: c1ccc(-n2c3ccccc3c3cc(-c4ccc5c(c4)c4ccccc4n5-c4ccccc4)ccc32)cc1] B-4 |

As seen from Tables 1 and 2 above, it is confirmed that the lifespan characteristic of Device Examples 1 to 4 is superior to that of the Comparative Examples due to higher anion stability of the second hole transport layer. That is, the problem of the decrease in lifespan followed by the increase of efficiency is overcome.

[Triplet]

The triplet energy was calculated by, first, conducting structure optimization in the ground state by applying 6-31G* basis set to B3LYP, which is one of the Density Functional Theory (DFT) methods, and then, TD-DFT calculation using the same basis set and the same theory in the optimized structure. In all the calculations, the program, Gaussian 03, was used.

[Determination of Structure]

The optimization of structure in the ground state was conducted by applying 6-31G* basis set to B3LYP, which is one of the DFT methods.

[Anion Stability]

The anion stability was calculated by conducting structure optimization in the ground state by applying 6-31G* basis set to B3LYP, which is one of the DFT methods, and then, reoptimization in an electron state of −1 by randomly adding one electron to the calculated ground state structure, and determining the energy difference between the ground state and the electron state of −1.

Herein, it is preferable that the anion stability is at least a positive number (0 Kcal/mol or higher).

In similar molecular structures, a compound having a higher anion stability value is stable for electrons.

The anion stability values of the compounds used in the second hole transport layer of the Device Examples and the Comparative Examples found are shown in Table 4 below.

TABLE 4

| Second hole | Anion stability |
|---|---|
| transport layer | value |
| B-1 | 0.416 |
| B-3 | −2.04 |
| B-4 | −7.56 |
| A-1 | 3.83 |
| A-4 | 0.548 |
| A-7 | 7.18 |
| A-15 | 5.42 |

1. A hole transport material comprising a compound represented by the following formula 1: 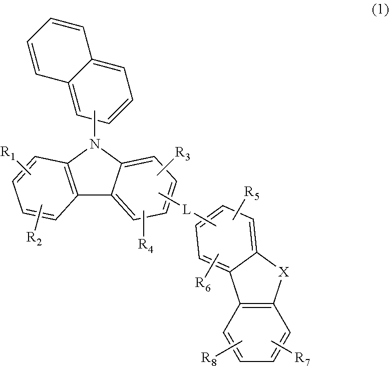
wherein
X represents O, S, CR₉R₁₀, or NR₁₁;
L represents a single bond, or a substituted or unsubstituted (C6-C30)arylene;
R₁ to R₁₁ each independently represent hydrogen, deuterium, a substituted or unsubstituted (C1-C30)alkyl, a substituted or unsubstituted (C6-C30)aryl, a substituted or unsubstituted 3- to 30-membered heteroaryl, a substituted or unsubstituted (C3-C30)cycloalkyl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or are linked to each other to form a mono- or polycyclic, (C3-C30) alicyclic or aromatic ring, whose carbon atom(s) may be replaced with at least one hetero atom selected from nitrogen, oxygen, and sulfur; and
the heteroaryl contains at least one hetero atom selected from B, N, O, S, Si, and P.

2. The hole transport material according to claim 1, wherein the substituents of the substituted (C1-C30)alkyl, the substituted (C3-C30)cycloalkyl, the substituted (C6-C30)aryl(ene), the substituted 3- to 30-membered heteroaryl, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di-(C1-C30)alkylamino, the substituted mono- or di-(C6-C30)arylamino, and the substituted (C1-C30)alkyl(C6-C30)arylamino in L, and R₁ to R₁₁ each independently are at least one selected from the group consisting of deuterium, a halogen, a cyano, a carboxyl, a nitro, a hydroxyl, a (C1-C30)alkyl, a halo(C1-C30)alkyl, a (C2-C30) alkenyl, a (C2-C30) alkynyl, a (C1-C30)alkoxy, a (C1-C30)alkylthio, a (C3-C30)cycloalkyl, a (C3-C30)cycloalkenyl, a 3- to 7-membered heterocycloalkyl, a (C6-C30)aryloxy, a (C6-C30)arylthio, a 3- to 30-membered heteroaryl unsubstituted or substituted with a (C6-C30)aryl, a (C6-C30)aryl unsubstituted or substituted with a 3- to 30-membered heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

3. The hole transport material according to claim 1, wherein
X represents O, S, CR₉R₁₀, or NR₁₁;
L represents a single bond, or a substituted or unsubstituted (C6-C12)arylene;
R₁ to R₈ each independently represent hydrogen, or a substituted or unsubstituted 5- to 15-membered heteroaryl; or are linked to each other to form a mono- or polycyclic, (C5-C15) alicyclic or aromatic ring; and
R₉ to R₁₁ each independently represent hydrogen, a substituted or unsubstituted (C1-C6)alkyl, or a substituted or unsubstituted (C6-C15)aryl; or are linked to each other to form a mono- or polycyclic, (C5-C15) alicyclic or aromatic ring.

4. The hole transport material according to claim 1, wherein
X represents O, S, CR₉R₁₀, or NR₁₁;
L represents a single bond, or an unsubstituted (C6-C12)arylene;
R₁ to R₈ each independently represent hydrogen, or a 5- to 15-membered heteroaryl unsubstituted or substituted with a (C6-C12)aryl; or are linked to each other to form a monocyclic, (C5-C15) aromatic ring; and
R₉ to R₁₁ each independently represent hydrogen, an unsubstituted (C1-C6)alkyl, or an unsubstituted (C6-C15)aryl; or are linked to each other to form a polycyclic, (C5-C15) aromatic ring.

5. The hole transport material according to claim 1, wherein the compound represented by formula 1 is selected from the group consisting of:

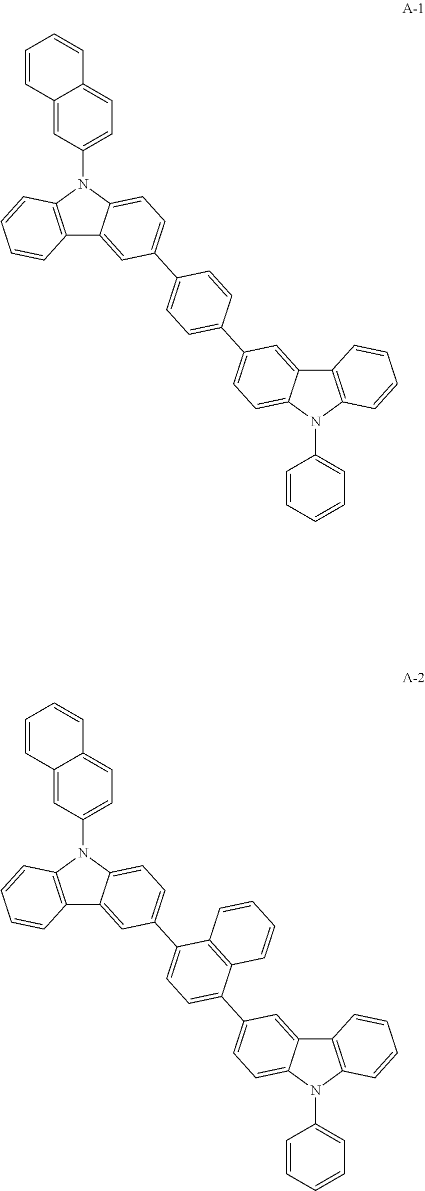 [SMILES: c1ccc(-n2c3ccccc3c3cc(-c4ccc(-c5ccc6c(c5)c5ccccc5n6-c5ccc6ccccc6c5)cc4)ccc32)cc1 ; c1ccc(-n2c3ccccc3c3cc(-c4ccc(-c5ccc6c(c5)c5ccccc5n6-c5ccc6ccccc6c5)c5ccccc45)ccc32)cc1]

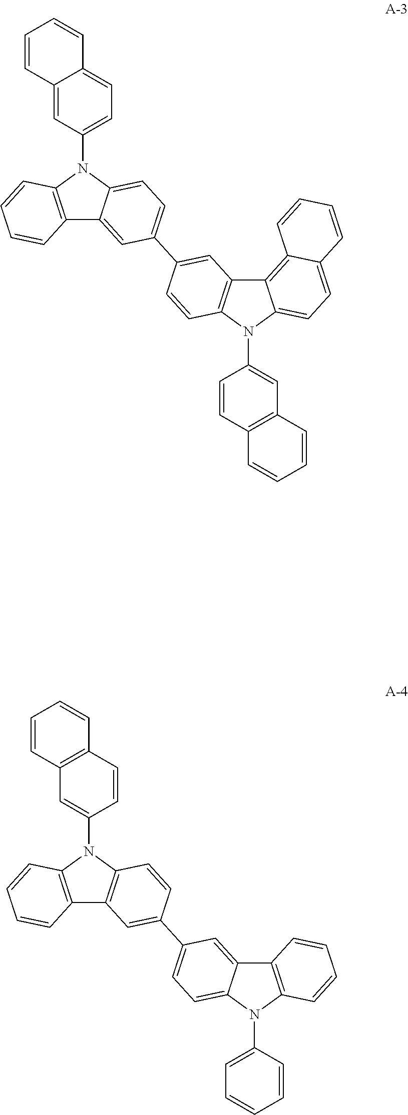 [SMILES: c1ccc2cc(-n3c4ccccc4c4cc(-c5ccc6c(c5)c5c7ccccc7ccc5n6-c5ccc6ccccc6c5)ccc43)ccc2c1 ; c1ccc(-n2c3ccccc3c3cc(-c4ccc5c(c4)c4ccccc4n5-c4ccc5ccccc5c4)ccc32)cc1]

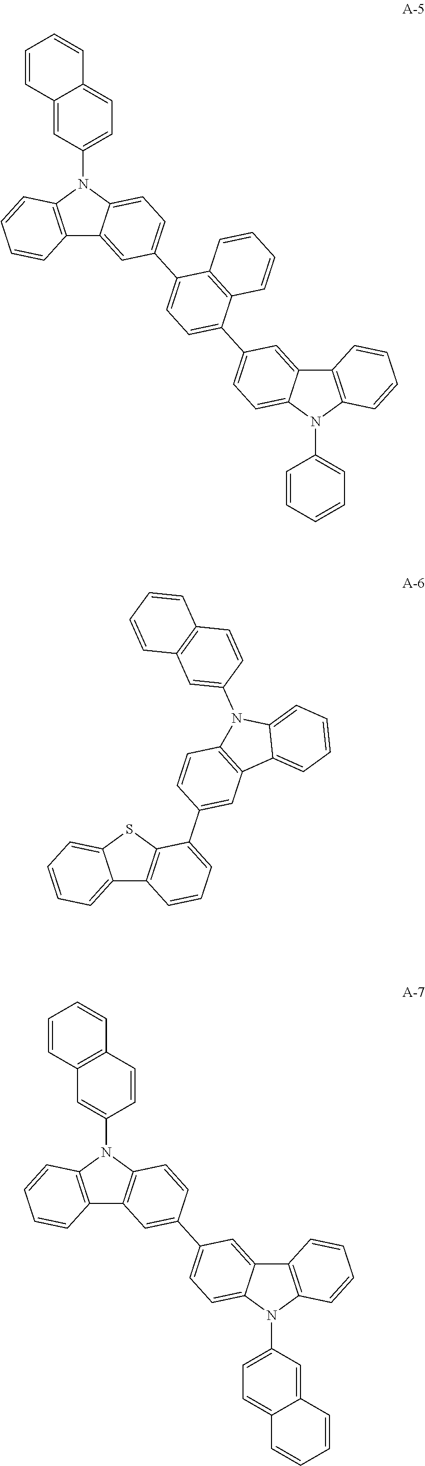 [SMILES: c1ccc(-n2c3ccccc3c3cc(-c4ccc(-c5ccc6c(c5)c5ccccc5n6-c5ccc6ccccc6c5)c5ccccc45)ccc32)cc1 ; c1ccc2cc(-n3c4ccccc4c4cc(-c5cccc6c5sc5ccccc56)ccc43)ccc2c1 ; c1ccc2cc(-n3c4ccccc4c4cc(-c5ccc6c(c5)c5ccccc5n6-c5ccc6ccccc6c5)ccc43)ccc2c1]

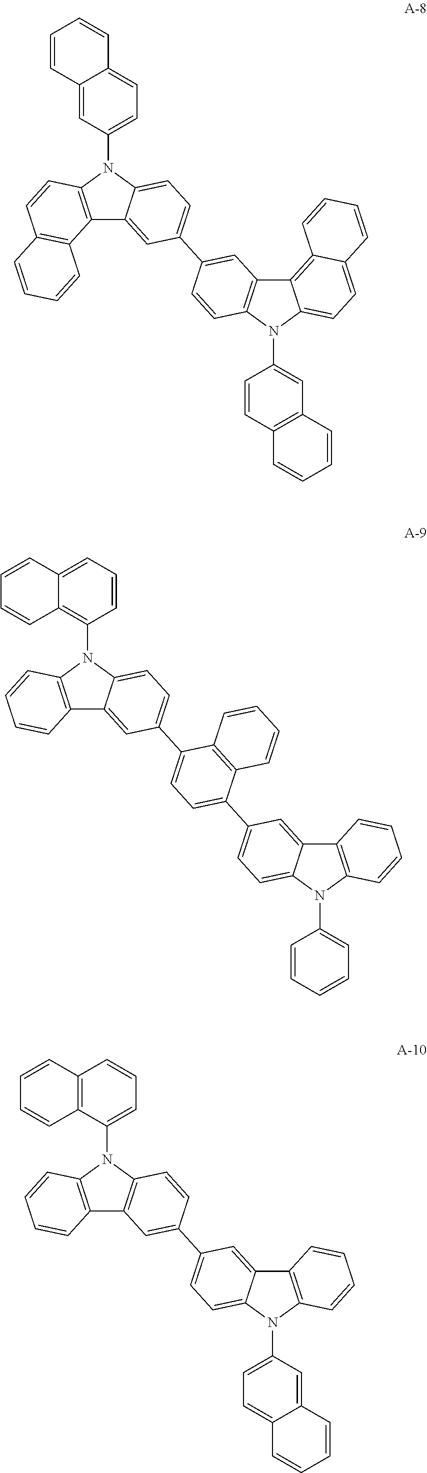 [SMILES: c1ccc2cc(-n3c4ccc(-c5ccc6c(c5)c5c7ccccc7ccc5n6-c5ccc6ccccc6c5)cc4c4c5ccccc5ccc43)ccc2c1 ; c1ccc(-n2c3ccccc3c3cc(-c4ccc(-c5ccc6c(c5)c5ccccc5n6-c5cccc6ccccc56)c5ccccc45)ccc32)cc1 ; c1ccc2cc(-n3c4ccccc4c4cc(-c5ccc6c(c5)c5ccccc5n6-c5cccc6ccccc56)ccc43)ccc2c1]

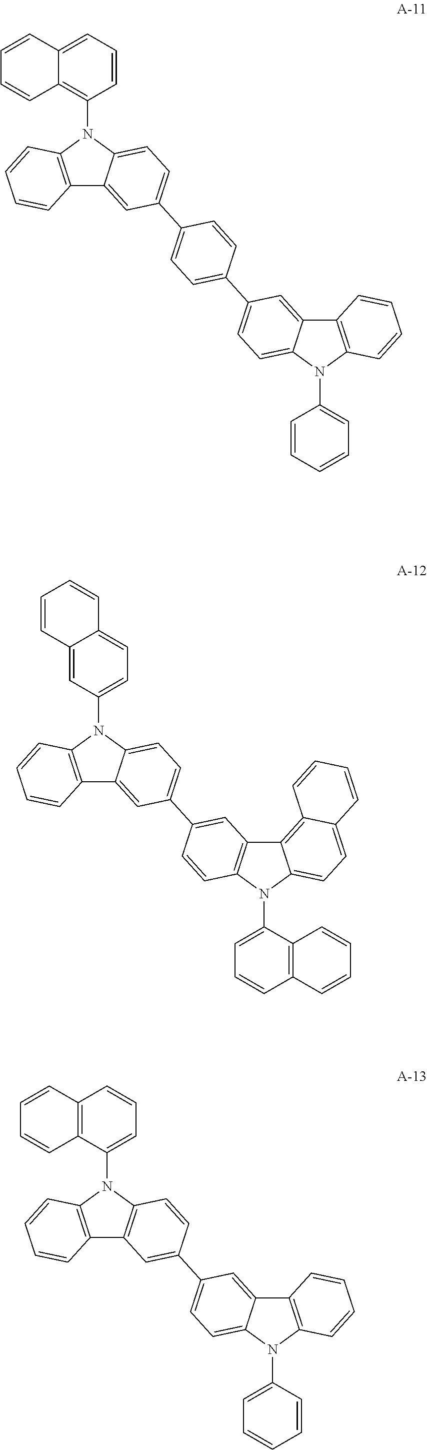 [SMILES: c1ccc(-n2c3ccccc3c3cc(-c4ccc(-c5ccc6c(c5)c5ccccc5n6-c5cccc6ccccc56)cc4)ccc32)cc1 ; c1ccc2cc(-n3c4ccccc4c4cc(-c5ccc6c(c5)c5c7ccccc7ccc5n6-c5cccc6ccccc56)ccc43)ccc2c1 ; c1ccc(-n2c3ccccc3c3cc(-c4ccc5c(c4)c4ccccc4n5-c4cccc5ccccc45)ccc32)cc1]

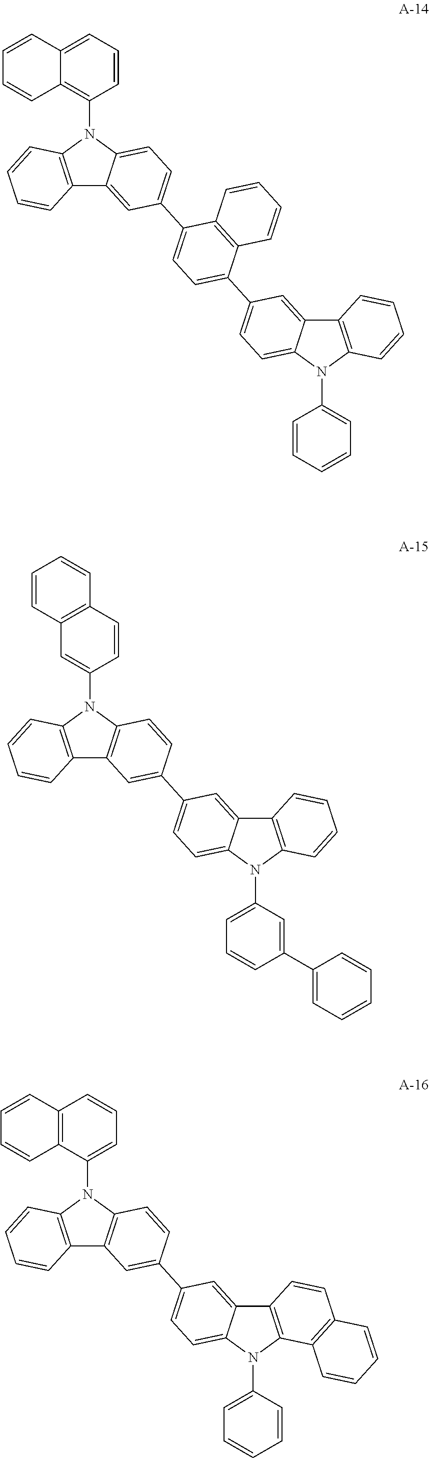 [SMILES: c1ccc(-n2c3ccccc3c3cc(-c4ccc(-c5ccc6c(c5)c5ccccc5n6-c5cccc6ccccc56)c5ccccc45)ccc32)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-n2c3ccc(-c4ccc5c(c4)c4ccccc4n5-c4cccc5ccccc45)cc3c3ccc4ccccc4c32)cc1]

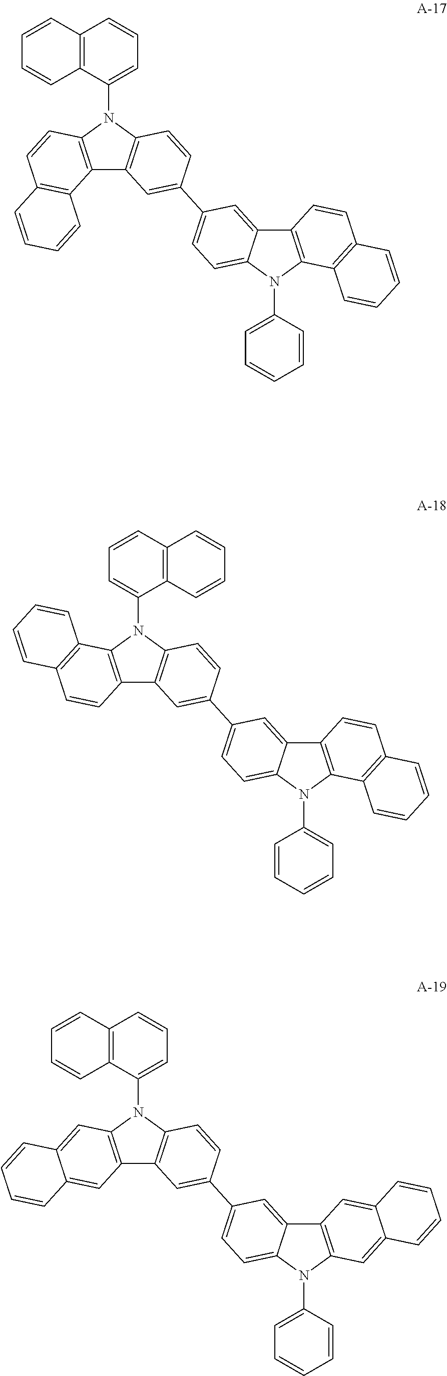 [SMILES: c1ccc(-n2c3ccc(-c4ccc5c(c4)c4c6ccccc6ccc4n5-c4cccc5ccccc45)cc3c3ccc4ccccc4c32)cc1 ; c1ccc(-n2c3ccc(-c4ccc5c(c4)c4ccc6ccccc6c4n5-c4cccc5ccccc45)cc3c3ccc4ccccc4c32)cc1 ; c1ccc(-n2c3ccc(-c4ccc5c(c4)c4cc6ccccc6cc4n5-c4cccc5ccccc45)cc3c3cc4ccccc4cc32)cc1]

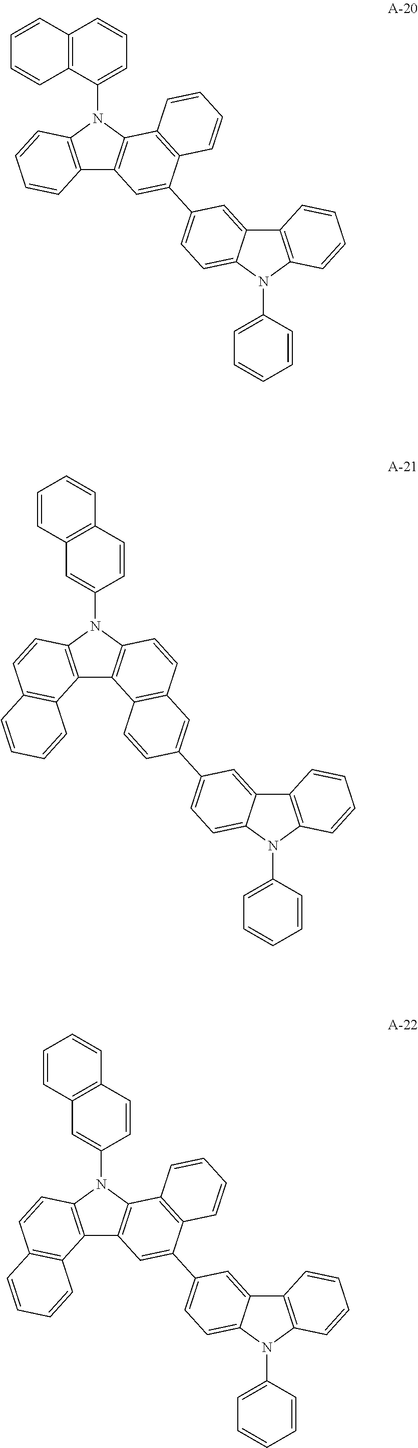 [SMILES: c1ccc(-n2c3ccccc3c3cc(-c4cc5c6ccccc6n(-c6cccc7ccccc67)c5c5ccccc45)ccc32)cc1 ; c1ccc(-n2c3ccccc3c3cc(-c4ccc5c(ccc6c5c5c7ccccc7ccc5n6-c5ccc6ccccc6c5)c4)ccc32)cc1 ; c1ccc(-n2c3ccccc3c3cc(-c4cc5c6c7ccccc7ccc6n(-c6ccc7ccccc7c6)c5c5ccccc45)ccc32)cc1]

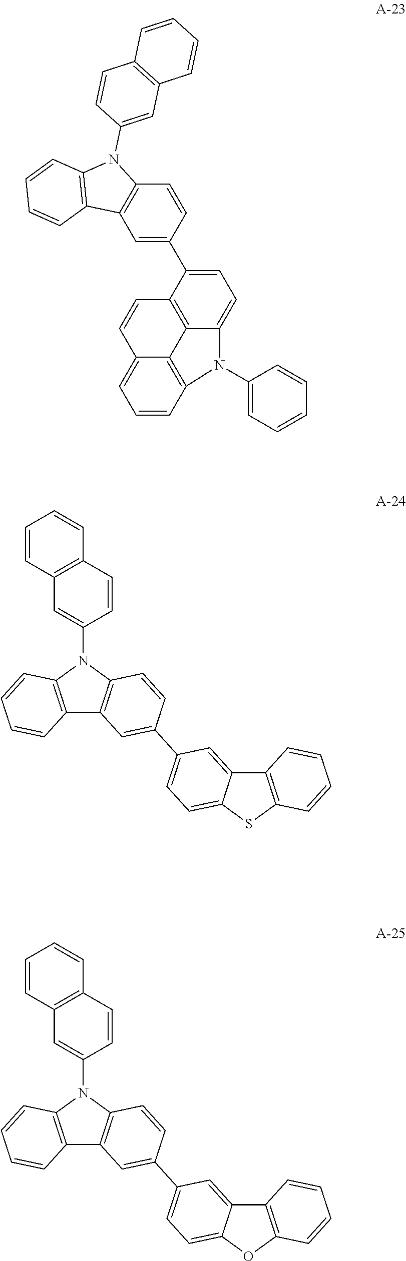 [SMILES: c1ccc(-n2c3cccc4ccc5c(-c6ccc7c(c6)c6ccccc6n7-c6ccc7ccccc7c6)ccc2c5c43)cc1 ; c1ccc2cc(-n3c4ccccc4c4cc(-c5ccc6sc7ccccc7c6c5)ccc43)ccc2c1 ; c1ccc2cc(-n3c4ccccc4c4cc(-c5ccc6oc7ccccc7c6c5)ccc43)ccc2c1]

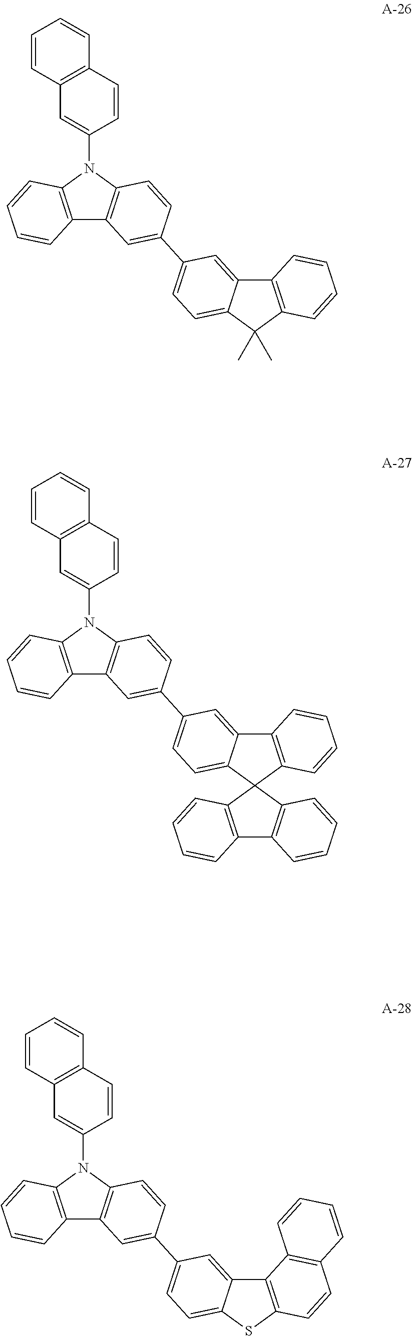 [SMILES: CC1(C)c2ccccc2-c2cc(-c3ccc4c(c3)c3ccccc3n4-c3ccc4ccccc4c3)ccc21 ; c1ccc2c(c1)-c1ccccc1C21c2ccccc2-c2cc(-c3ccc4c(c3)c3ccccc3n4-c3ccc4ccccc4c3)ccc21 ; c1ccc2cc(-n3c4ccccc4c4cc(-c5ccc6sc7ccc8ccccc8c7c6c5)ccc43)ccc2c1]

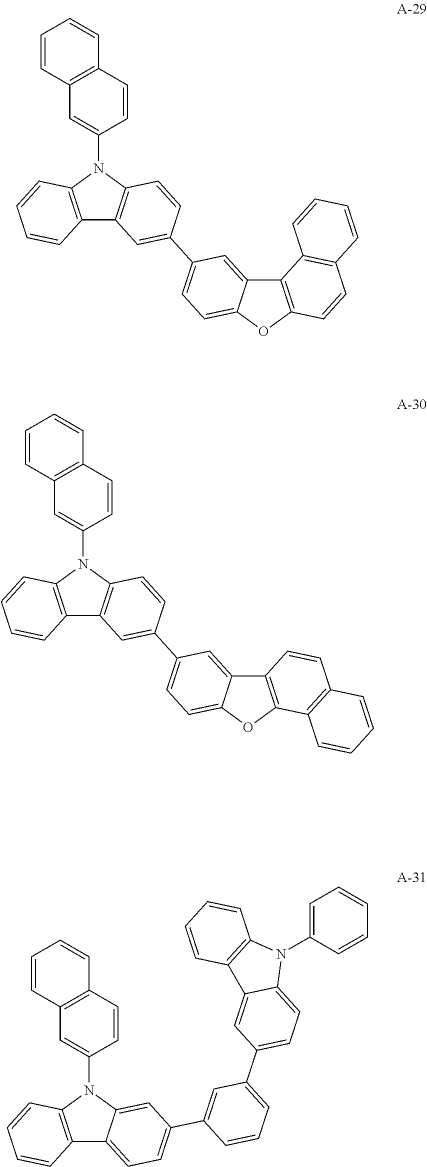 [SMILES: c1ccc2cc(-n3c4ccccc4c4cc(-c5ccc6oc7ccc8ccccc8c7c6c5)ccc43)ccc2c1 ; c1ccc2cc(-n3c4ccccc4c4cc(-c5ccc6oc7c8ccccc8ccc7c6c5)ccc43)ccc2c1 ; c1ccc(-n2c3ccccc3c3cc(-c4cccc(-c5ccc6c7ccccc7n(-c7ccc8ccccc8c7)c6c5)c4)ccc32)cc1]

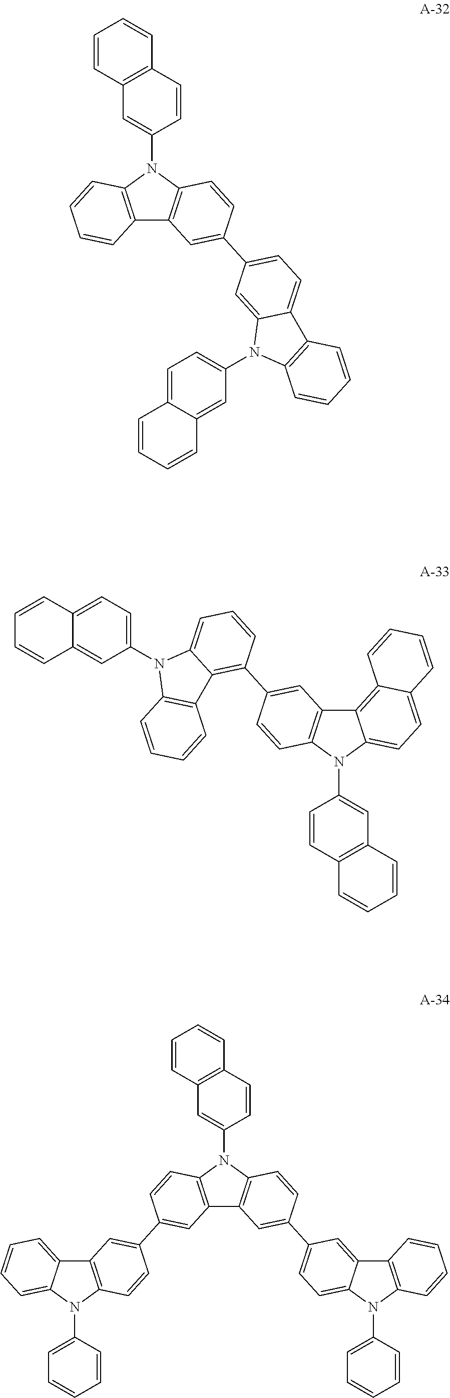 [SMILES: c1ccc2cc(-n3c4ccccc4c4cc(-c5ccc6c7ccccc7n(-c7ccc8ccccc8c7)c6c5)ccc43)ccc2c1 ; c1ccc2cc(-n3c4ccccc4c4c(-c5ccc6c(c5)c5c7ccccc7ccc5n6-c5ccc6ccccc6c5)cccc43)ccc2c1 ; c1ccc(-n2c3ccccc3c3cc(-c4ccc5c(c4)c4cc(-c6ccc7c(c6)c6ccccc6n7-c6ccccc6)ccc4n5-c4ccc5ccccc5c4)ccc32)cc1]

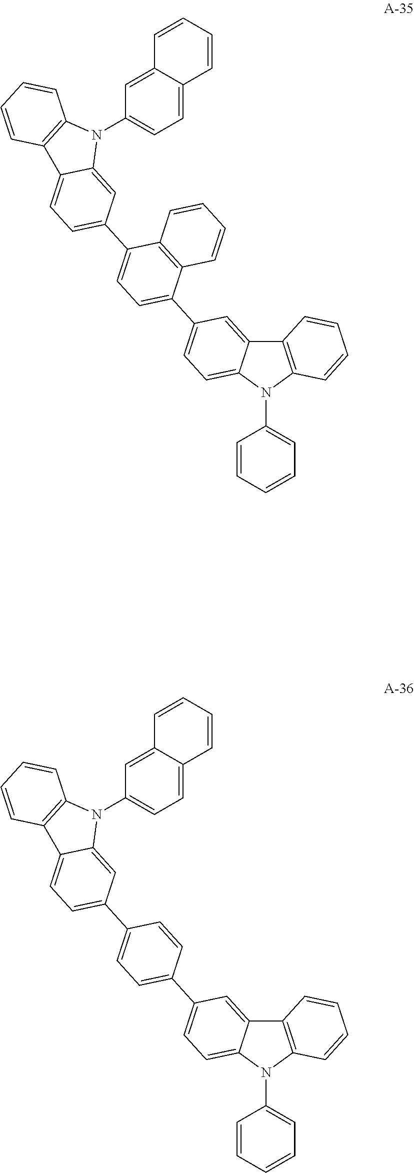 [SMILES: c1ccc(-n2c3ccccc3c3cc(-c4ccc(-c5ccc6c7ccccc7n(-c7ccc8ccccc8c7)c6c5)c5ccccc45)ccc32)cc1 ; c1ccc(-n2c3ccccc3c3cc(-c4ccc(-c5ccc6c7ccccc7n(-c7ccc8ccccc8c7)c6c5)cc4)ccc32)cc1]

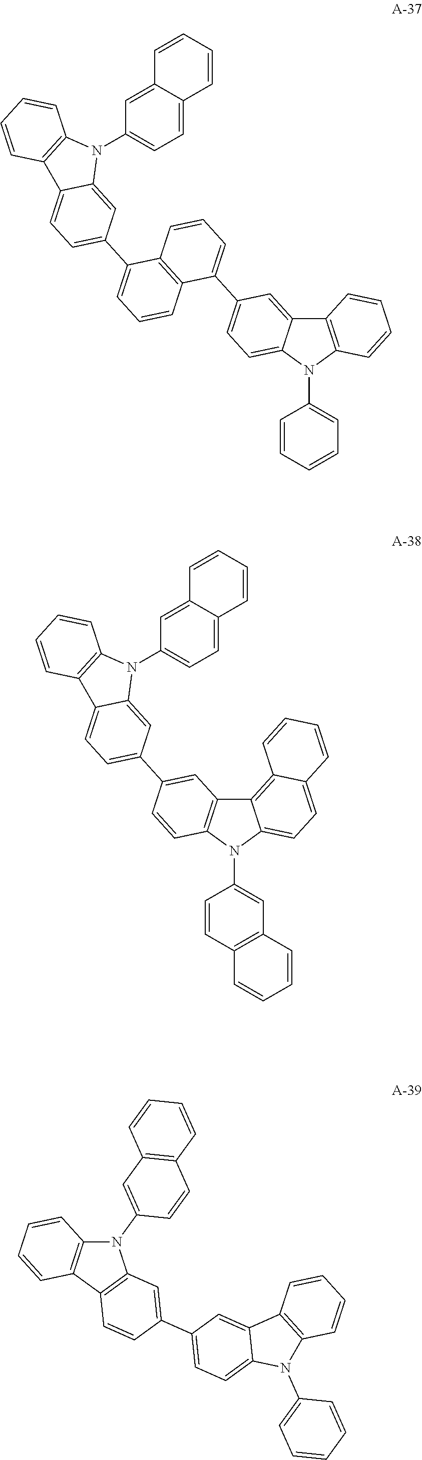 [SMILES: c1ccc(-n2c3ccccc3c3cc(-c4cccc5c(-c6ccc7c8ccccc8n(-c8ccc9ccccc9c8)c7c6)cccc45)ccc32)cc1 ; c1ccc2cc(-n3c4ccccc4c4ccc(-c5ccc6c(c5)c5c7ccccc7ccc5n6-c5ccc6ccccc6c5)cc43)ccc2c1 ; c1ccc(-n2c3ccccc3c3cc(-c4ccc5c6ccccc6n(-c6ccc7ccccc7c6)c5c4)ccc32)cc1]

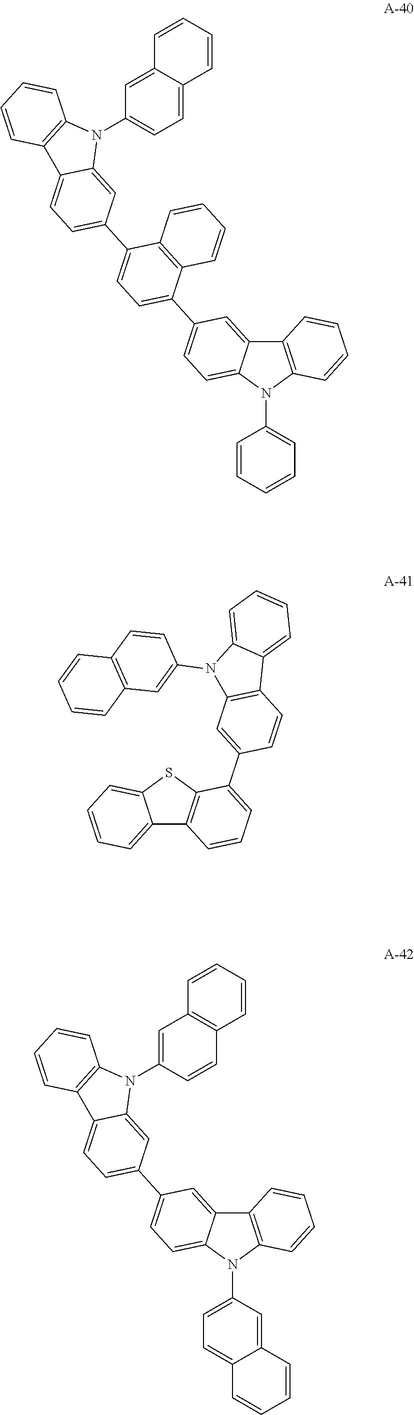 [SMILES: c1ccc(-n2c3ccccc3c3cc(-c4ccc(-c5ccc6c7ccccc7n(-c7ccc8ccccc8c7)c6c5)c5ccccc45)ccc32)cc1 ; c1ccc2cc(-n3c4ccccc4c4ccc(-c5cccc6c5sc5ccccc56)cc43)ccc2c1 ; c1ccc2cc(-n3c4ccccc4c4cc(-c5ccc6c7ccccc7n(-c7ccc8ccccc8c7)c6c5)ccc43)ccc2c1]

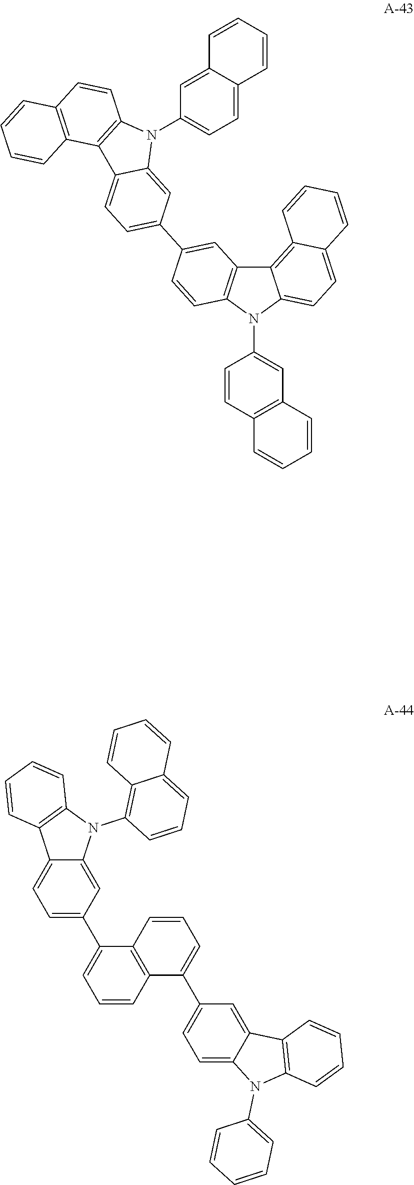 [SMILES: c1ccc2cc(-n3c4ccc(-c5ccc6c7c8ccccc8ccc7n(-c7ccc8ccccc8c7)c6c5)cc4c4c5ccccc5ccc43)ccc2c1 ; c1ccc(-n2c3ccccc3c3cc(-c4cccc5c(-c6ccc7c8ccccc8n(-c8cccc9ccccc89)c7c6)cccc45)ccc32)cc1]

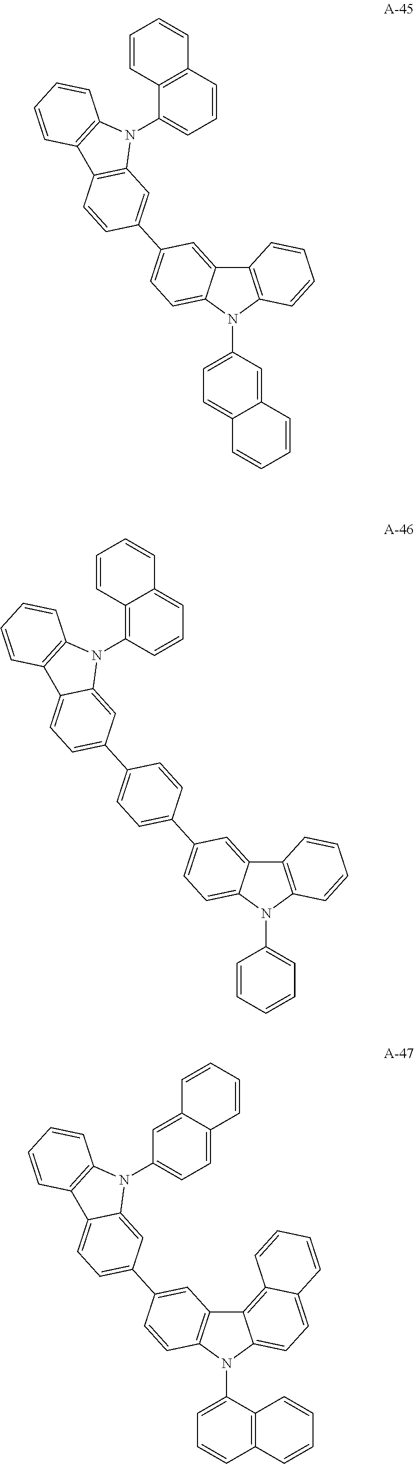 [SMILES: c1ccc2cc(-n3c4ccccc4c4cc(-c5ccc6c7ccccc7n(-c7cccc8ccccc78)c6c5)ccc43)ccc2c1 ; c1ccc(-n2c3ccccc3c3cc(-c4ccc(-c5ccc6c7ccccc7n(-c7cccc8ccccc78)c6c5)cc4)ccc32)cc1 ; c1ccc2cc(-n3c4ccccc4c4ccc(-c5ccc6c(c5)c5c7ccccc7ccc5n6-c5cccc6ccccc56)cc43)ccc2c1]

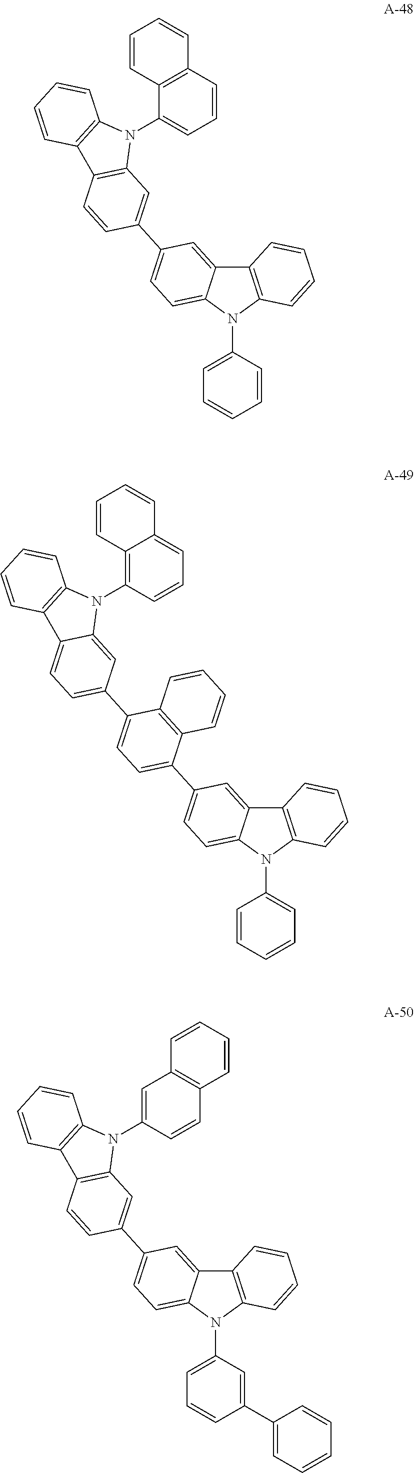 [SMILES: c1ccc(-n2c3ccccc3c3cc(-c4ccc5c6ccccc6n(-c6cccc7ccccc67)c5c4)ccc32)cc1 ; c1ccc(-n2c3ccccc3c3cc(-c4ccc(-c5ccc6c7ccccc7n(-c7cccc8ccccc78)c6c5)c5ccccc45)ccc32)cc1 ; c1ccc(-c2cccc(-n3c4ccccc4c4cc(-c5ccc6c7ccccc7n(-c7ccc8ccccc8c7)c6c5)ccc43)c2)cc1]

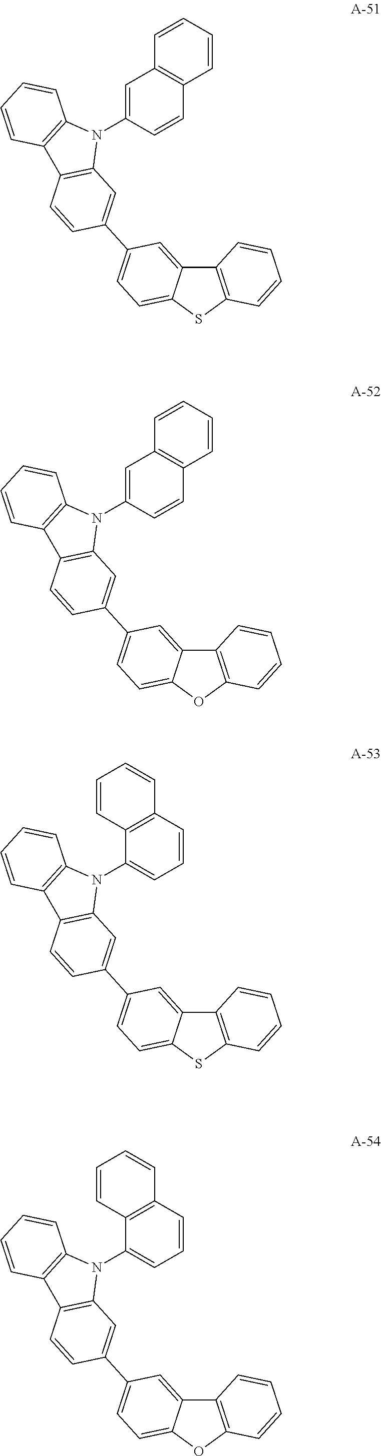 [SMILES: c1ccc2cc(-n3c4ccccc4c4ccc(-c5ccc6sc7ccccc7c6c5)cc43)ccc2c1 ; c1ccc2cc(-n3c4ccccc4c4ccc(-c5ccc6oc7ccccc7c6c5)cc43)ccc2c1 ; c1ccc2c(-n3c4ccccc4c4ccc(-c5ccc6sc7ccccc7c6c5)cc43)cccc2c1 ; c1ccc2c(-n3c4ccccc4c4ccc(-c5ccc6oc7ccccc7c6c5)cc43)cccc2c1]

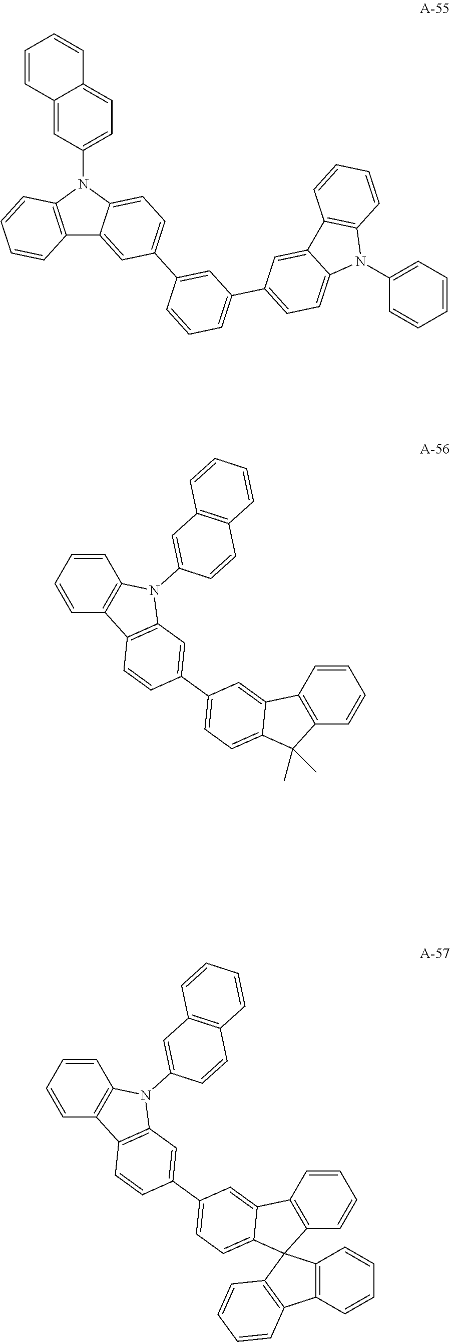 [SMILES: c1ccc(-n2c3ccccc3c3cc(-c4cccc(-c5ccc6c(c5)c5ccccc5n6-c5ccc6ccccc6c5)c4)ccc32)cc1 ; CC1(C)c2ccccc2-c2cc(-c3ccc4c5ccccc5n(-c5ccc6ccccc6c5)c4c3)ccc21 ; c1ccc2c(c1)-c1ccccc1C21c2ccccc2-c2cc(-c3ccc4c5ccccc5n(-c5ccc6ccccc6c5)c4c3)ccc21]

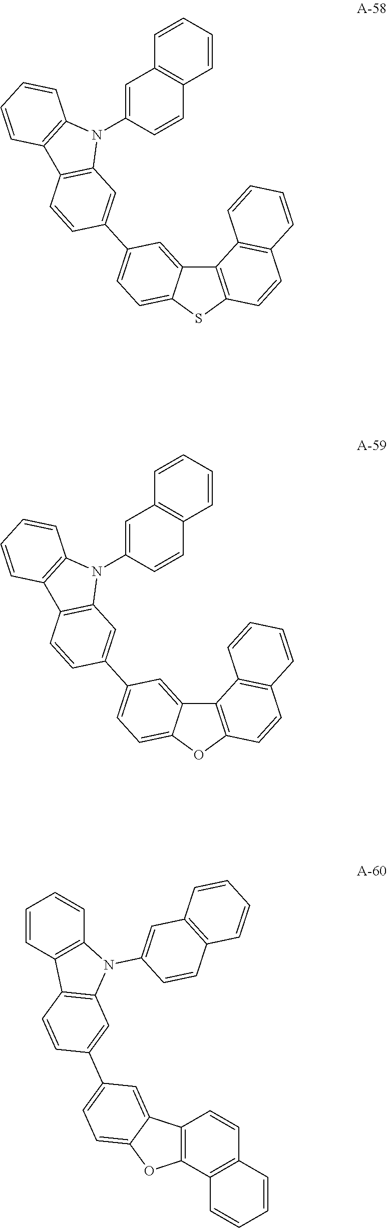 [SMILES: c1ccc2cc(-n3c4ccccc4c4ccc(-c5ccc6sc7ccc8ccccc8c7c6c5)cc43)ccc2c1 ; c1ccc2cc(-n3c4ccccc4c4ccc(-c5ccc6oc7ccc8ccccc8c7c6c5)cc43)ccc2c1 ; c1ccc2cc(-n3c4ccccc4c4ccc(-c5ccc6oc7c8ccccc8ccc7c6c5)cc43)ccc2c1]

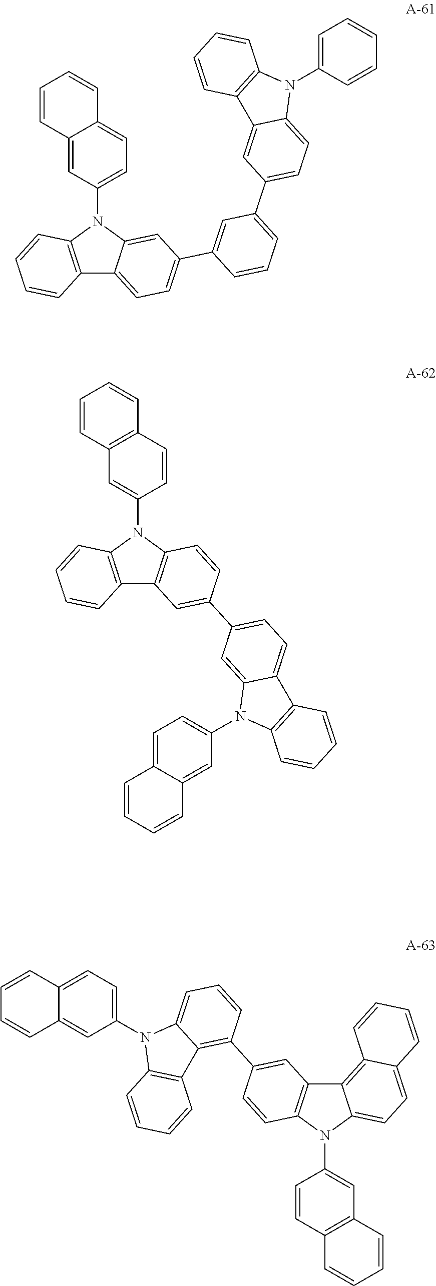 [SMILES: c1ccc(-n2c3ccccc3c3cc(-c4cccc(-c5ccc6c7ccccc7n(-c7ccc8ccccc8c7)c6c5)c4)ccc32)cc1 ; c1ccc2cc(-n3c4ccccc4c4cc(-c5ccc6c7ccccc7n(-c7ccc8ccccc8c7)c6c5)ccc43)ccc2c1 ; c1ccc2cc(-n3c4ccccc4c4c(-c5ccc6c(c5)c5c7ccccc7ccc5n6-c5ccc6ccccc6c5)cccc43)ccc2c1]

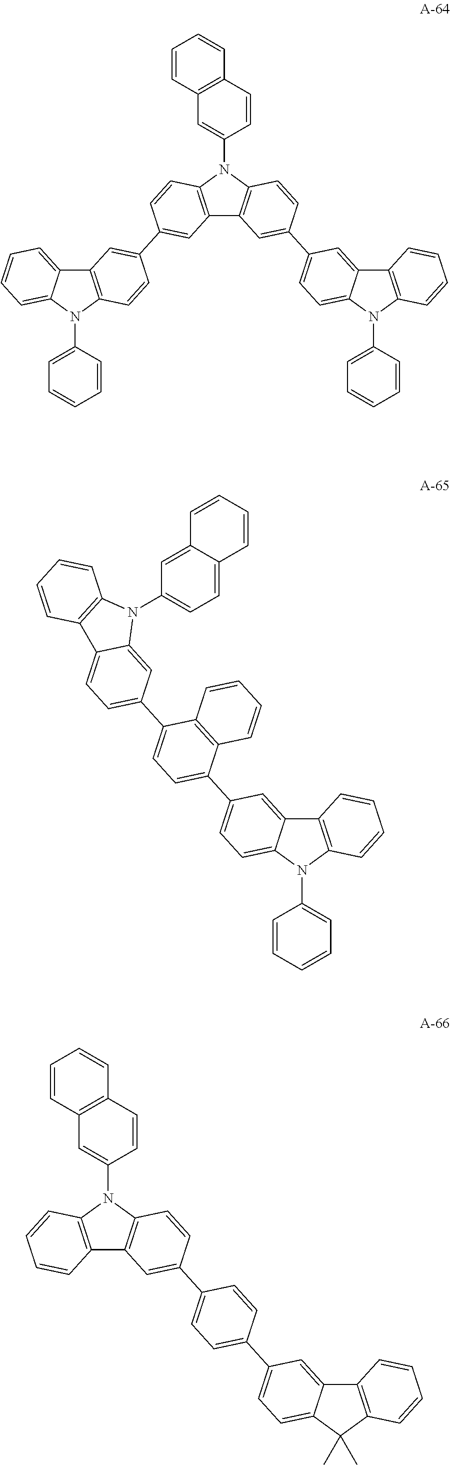 [SMILES: c1ccc(-n2c3ccccc3c3cc(-c4ccc5c(c4)c4cc(-c6ccc7c(c6)c6ccccc6n7-c6ccccc6)ccc4n5-c4ccc5ccccc5c4)ccc32)cc1 ; c1ccc(-n2c3ccccc3c3cc(-c4ccc(-c5ccc6c7ccccc7n(-c7ccc8ccccc8c7)c6c5)c5ccccc45)ccc32)cc1 ; CC1(C)c2ccccc2-c2cc(-c3ccc(-c4ccc5c(c4)c4ccccc4n5-c4ccc5ccccc5c4)cc3)ccc21]

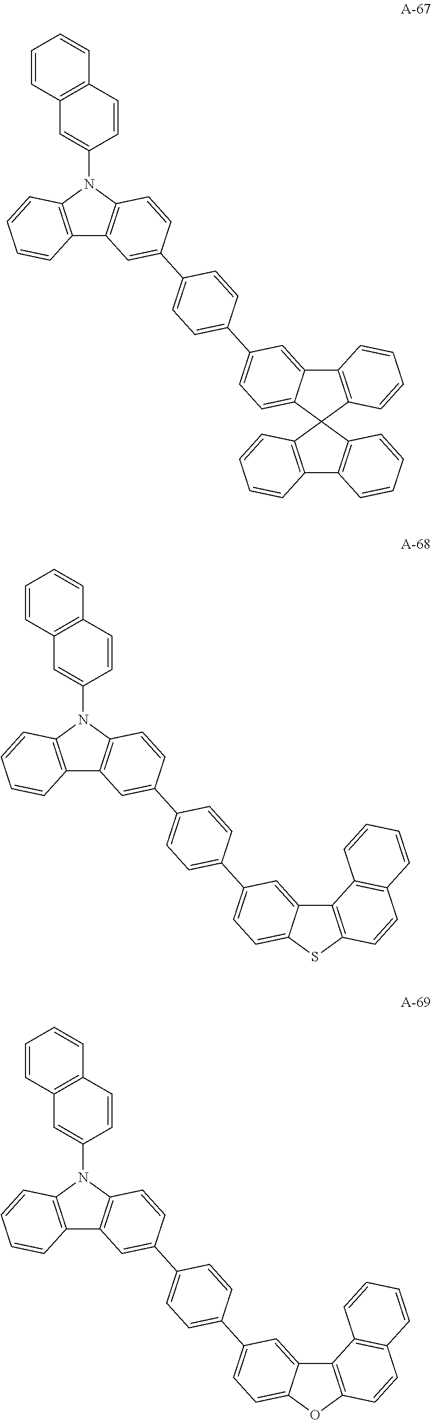 [SMILES: c1ccc2c(c1)-c1ccccc1C21c2ccccc2-c2cc(-c3ccc(-c4ccc5c(c4)c4ccccc4n5-c4ccc5ccccc5c4)cc3)ccc21 ; c1ccc2cc(-n3c4ccccc4c4cc(-c5ccc(-c6ccc7sc8ccc9ccccc9c8c7c6)cc5)ccc43)ccc2c1 ; c1ccc2cc(-n3c4ccccc4c4cc(-c5ccc(-c6ccc7oc8ccc9ccccc9c8c7c6)cc5)ccc43)ccc2c1]

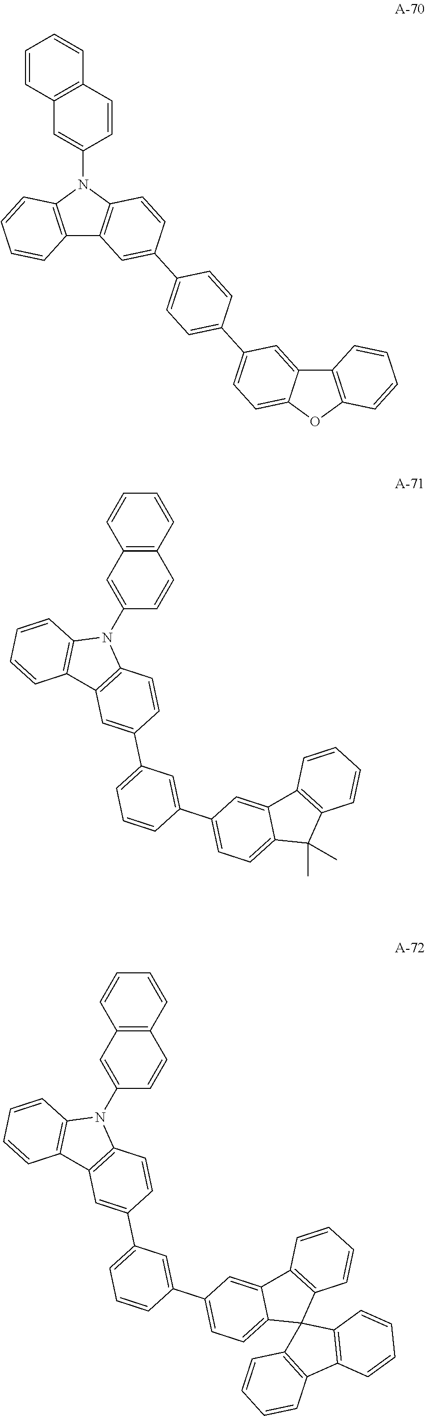 [SMILES: c1ccc2cc(-n3c4ccccc4c4cc(-c5ccc(-c6ccc7oc8ccccc8c7c6)cc5)ccc43)ccc2c1 ; CC1(C)c2ccccc2-c2cc(-c3cccc(-c4ccc5c(c4)c4ccccc4n5-c4ccc5ccccc5c4)c3)ccc21 ; c1cc(-c2ccc3c(c2)-c2ccccc2C32c3ccccc3-c3ccccc32)cc(-c2ccc3c(c2)c2ccccc2n3-c2ccc3ccccc3c2)c1]

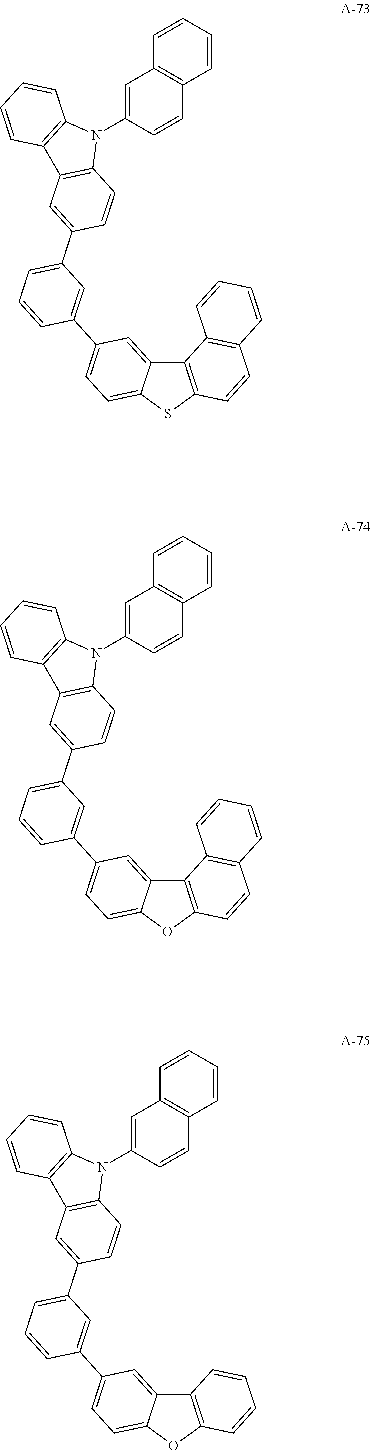 [SMILES: c1cc(-c2ccc3sc4ccc5ccccc5c4c3c2)cc(-c2ccc3c(c2)c2ccccc2n3-c2ccc3ccccc3c2)c1 ; c1cc(-c2ccc3oc4ccc5ccccc5c4c3c2)cc(-c2ccc3c(c2)c2ccccc2n3-c2ccc3ccccc3c2)c1 ; c1cc(-c2ccc3oc4ccccc4c3c2)cc(-c2ccc3c(c2)c2ccccc2n3-c2ccc3ccccc3c2)c1]

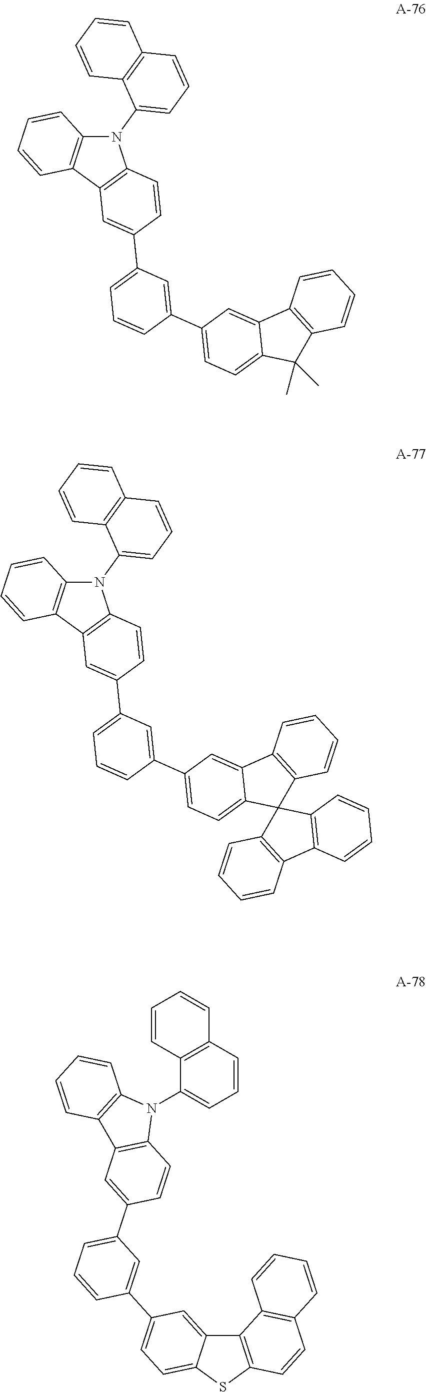 [SMILES: CC1(C)c2ccccc2-c2cc(-c3cccc(-c4ccc5c(c4)c4ccccc4n5-c4cccc5ccccc45)c3)ccc21 ; c1cc(-c2ccc3c(c2)-c2ccccc2C32c3ccccc3-c3ccccc32)cc(-c2ccc3c(c2)c2ccccc2n3-c2cccc3ccccc23)c1 ; c1cc(-c2ccc3sc4ccc5ccccc5c4c3c2)cc(-c2ccc3c(c2)c2ccccc2n3-c2cccc3ccccc23)c1]

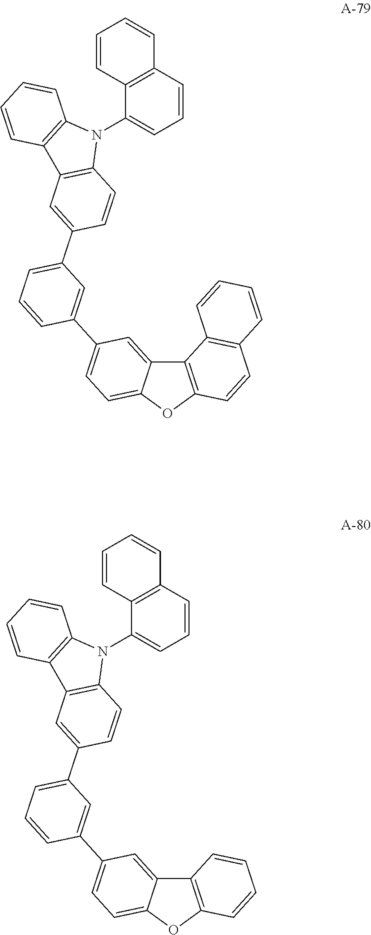 [SMILES: c1cc(-c2ccc3oc4ccc5ccccc5c4c3c2)cc(-c2ccc3c(c2)c2ccccc2n3-c2cccc3ccccc23)c1 ; c1cc(-c2ccc3oc4ccccc4c3c2)cc(-c2ccc3c(c2)c2ccccc2n3-c2cccc3ccccc23)c1]

6. An organic electroluminescent device comprising the hole transport material according to claim 1.
